(19) United States
(12) Patent Application Publication
(10) Pub. No.: US 2023/0256085 A1
(43) Pub. Date: Aug. 17, 2023

(54) COMBINATION VACCINE

(71) Applicant: SPICONA INC., New York, NY (US)

(72) Inventors: Reinhard GLUCK, Bern (CH); Agata FAZIO, Catania (IT); Emanuele MONTOMOLI, Siena (IT); Kaspar BANZIGER, Pfeffingen (CH); Carlos QUINTO, Basel (CH)

(21) Appl. No.: 18/015,025

(22) PCT Filed: Jul. 7, 2021
(86) PCT No.: PCT/IB2021/056102

§ 371 Date: Jan. 6, 2023

(30) Foreign Application Priority Data
Jul. 7, 2020 (GB) 2010425.3

(51) Int. Cl. A61K 39/215; A61K 39/145; A61K 39/29; A61K 39/12; A61P 31/14

(52) U.S. Cl. CPC A61K 39/215; A61K 39/145; A61K 39/292; A61K 39/12; A61P 31/14; A61K 2039/70

(57) ABSTRACT

The present invention relates to combination vaccines against both influenza and COVID-19. In particular, the invention relates to combination vaccines comprising one or more influenza virus antigen and one or more SARS-CoV-2 (Coronavirus SARS-CoV-2) antigen, particularly one or more SARS-CoV-2 spike protein antigen, as well as vaccines comprising polynucleotides encoding said antigens, and such vaccines for the treatment or prevention of COVID-19 (SARS-CoV-2 infection) and influenza infection.

### FIELD OF THE INVENTION

The present invention relates to combination vaccines against both influenza and COVID-19. In particular, the invention relates to combination vaccines comprising one or more influenza virus antigen and one or more SARS-CoV-2 antigen, preferably at least one SARS-CoV-2(Coronavirus 2019-nCoV) spike protein antigen, as well as vaccines comprising polynucleotides encoding said antigens, and such vaccines for the treatment or prevention of COVID-19 (SARS-CoV-2 infection) and influenza infection.

### BACKGROUND OF THE INVENTION

As of 29 Jun. 2020, over 10,000,000 people were confirmed as positive for COVID-19 (the disease caused by severe acute respiratory syndrome coronavirus 2 (SARS-CoV-2, or Coronavirus 2019-nCoV) worldwide. By this same date, over 500,000 deaths had recorded globally due to COVID-19.

The majority of patients infected with SARS-CoV-2 experience mild to moderate symptoms include a high temperature or fever, a cough, shortness of breath, fatigue, and a loss or change to an individual's sense of smell or taste. Some patients progress to severe disease, which may involve acute respiratory distress syndrome (ARDS), cytokine storm, multi-organ failure, septic shock, and blood clots. In addition, some patients who test positive for SARS-CoV-2 infection are asymptomatic, or experience minimal symptoms, making diagnosis difficult unless a test is carried out. The evidence to-date indicates that these asymptomatic patients shed SARS-CoV-2 viral particles (often for longer than patients with symptomatic infection), and so can still efficiently spread the SARS-CoV-2 virus.

The wide range in symptoms associated with SARS-CoV-2 infection, and the existence of asymptomatic patients makes determining the epidemiological characteristics of COVID-19 more difficult. In addition, at least one study indicates that the majority of both asymptomatic and symptomatic patients had reduced levels of IgG and neutralising antibodies against SARS-CoV-2 as little as eight weeks into convalescence. Some clinical data demonstrates that significant proportion of asymptomatic patients (40%), as well as smaller numbers of patients with symptomatic infections (˜13%) are seronegative for IgG in early convalescence (Long et al. Nat. Med. 2020 https://doi.org/10.1038/s41591-020-0965-6). Therefore, whilst the development of a vaccine for SARS-CoV-2 is the subject of a vast global research drive, the available evidence suggests that any resulting immunity to SARS-CoV-2 infection is likely to be short-term in nature. Therefore, there is an ongoing need for the development of vaccines for COVID-19 which may be used in vaccines to generate and maintain protective immunity against SARS-CoV-2 infection and COVID-19 disease. Further, there is a need to provide vaccines which can be readily integrated into existing public health vaccination programs and schedules (factoring in issues relating to vaccine component suppression), and to produce such vaccines at scale and inexpensively.

The present invention addresses one or more of the above needs by providing combined influenza-COVID-19 vaccines. These combined vaccines comprise one or more influenza virus antigen and one or more SARS-CoV-2 antigen, preferably at least one SARS-CoV-2(Coronavirus 2019-nCoV) spike protein antigen, or one or more polynucleotide encoding said antigens, allowing for annual boosting of immunity against SARS-CoV-2 using existing public health programs already in place for influenza virus.

### SUMMARY OF THE INVENTION

To-date, whilst there are numerous vaccines for SARS-CoV-2 under development and/or in clinical trials, there is no approved vaccine available for general use. Furthermore, the available evidence suggests that immunity against SARS-CoV-2 may be relatively short-lived.

The present inventors have previously developed polynucleotides encoding the SARS-CoV-2 spike protein, said polynucleotides providing increased level and duration of expression of the SARS-CoV-2 spike protein, whilst retaining the conformation of the native spike protein.

The present inventors have now demonstrated that vaccine compositions comprising their SARS-CoV-2 spike protein can be successfully combined with influenza virus vaccines, with none of the expected problems of vaccine component suppression which are common in the production of combination vaccine products. In addition, whilst standard influenza vaccines do not contain an adjuvant, the adjuvant Addavax® can be successfully incorporated into a SARS-CoV-2/influenza vaccine according to the present invention. Enabling annual vaccination against SARS-CoV-2 infection within the existing public heath vaccine programs for influenza has the potential to boost immunity against SARS-CoV-2 whilst achieving good patient compliance.

Accordingly, the present invention provides a combined influenza-COVID-19 vaccine comprising: (a) an influenza haemagglutinin (HA) or an immunogenic fragment thereof; and (b) one or more antigen derived from SARS-CoV-2 or an immunogenic fragment thereof; wherein the antigens are capable of eliciting immune response and protection against both influenza and COVID-19.

Said combined influenza-COVID-19 vaccine may further comprise an influenza neuraminidase (NA) or an immunogenic fragment thereof. The influenza HA or immunogenic fragment thereof may be: (i) comprised in an inactivated influenza virion; (ii) a recombinant HA or immunogenic fragment thereof; (iii) a fusion protein comprising HA or an immunogenic fragment thereof; or (iv) encoded by an RNA or DNA vaccine. The influenza NA or immunogenic fragment thereof may be: (i) comprised in an inactivated influenza virion; (ii) a recombinant NA or immunogenic fragment thereof; (iii) a fusion protein comprising NA or an immunogenic fragment thereof; or (iv) encoded by an RNA or DNA vaccine. The one or more antigen derived from SARS-CoV-2 or an immunogenic fragment thereof may be: (i) at least one recombinant SARS-CoV-2 spike protein or immunogenic fragment thereof; (ii) at least one fusion protein comprising a SARS-CoV-2 spike protein or immunogenic fragment thereof; (iii) at least one virus-like particle (VLP) comprising a SARS-CoV-2 spike protein or immunogenic fragment thereof; (iv) at least one polynucleotide encoding a recombinant SARS-CoV-2 spike protein or immunogenic fragment thereof; or (v) encoded by at least one RNA or DNA vaccine.

In a combined influenza-COVID-19 vaccine of the invention (i) the influenza HA or immunogenic fragment thereof and the influenza NA or immunogenic fragment thereof may be comprised in an inactivated influenza virion; and (ii) the one or more antigen derived from SARS-CoV-2 or an immunogenic fragment thereof may be: (i) at least one fusion protein comprising a SARS-CoV-2 spike protein or immunogenic fragment thereof or (ii) at least one virus-like particle (VLP) comprising a SARS-CoV-2 spike protein or immunogenic fragment thereof.

In a combined influenza-COVID-19 vaccine of the invention: (a) the influenza HA or immunogenic fragment thereof may be comprised in a live attenuated influenza virion; (b) the influenza NA or immunogenic fragment thereof may be comprised in a live attenuated influenza virion; and/or (c) the one or more antigen derived from SARS-CoV-2 or an immunogenic fragment thereof may be comprised in a live viral vector. Said live viral vector comprising the one or more antigen derived from SARS-CoV-2 or an immunogenic fragment thereof may be: an adenoviral vector; a measles virus vector; a mumps virus vector; a rubella virus vector; a varicella virus vector; a polio virus vector; or a yellow fever virus vector.

A combined influenza-COVID-19 vaccine of the invention may, further comprising an adjuvant. Said adjuvant is typically stimulator of cellular (Th1) and/or humoral (Th2) immune responses, preferably both. Said adjuvant may comprise a squalene oil-in-water emulsion, an aluminium salt or a monophosphoryl Lipid A (MPL).

The one or more antigen derived from SARS-CoV-2 may be selected from: (a) a spike protein from SARS-CoV-2 having at least 90% identity with SEQ ID NO: 1, or a fragment thereof that has a common antigenic cross-reactivity with said spike protein; (b) a fusion protein comprising a spike protein from SARS-CoV-2 having at least 90% identity with SEQ ID NO: 1, or a fragment thereof that has a common antigenic cross-reactivity with said spike protein; (c) a VLP comprising a spike protein from SARS-CoV-2 having at least 90% identity with SEQ ID NO: 1, or a fragment thereof that has a common antigenic cross-reactivity with said spike protein; (d) a polynucleotide encoding a spike protein from SARS-CoV-2 having at least 90% identity with SEQ ID NO: 1, or a fragment thereof that has a common antigenic cross-reactivity with said spike protein; or (e) a viral vector, RNA vaccine or DNA plasmid that expresses a spike protein from SARS-CoV-2 having at least 90% identity with SEQ ID NO: 1, or a fragment thereof, that has a common antigenic cross-reactivity with said spike protein; wherein optionally the fragment of the SARS-CoV-2 spike protein comprises or consists of the receptor-binding domain (RBD) of the SARS-CoV-2 spike protein, preferably having at least 90% identity with SEQ ID NO: 15.

The one or more antigen derived from SARS-CoV-2 may be a fusion protein comprising a SARS-CoV-2 spike protein or immunogenic fragment thereof and further comprising: (a) the Hepatitis B surface antigen, or a fragment thereof that has a common antigenic cross-reactivity with said Hepatitis B surface antigen; (b) the HPV 18 L1 protein, or a fragment thereof that has a common antigenic cross-reactivity with said HPV 18 L1 protein; (c) the Hepatitis E P239 protein, or a fragment thereof that has a common antigenic cross-reactivity with said Hepatitis E P239 protein; and/or (e) the HPV 16 L1 protein, or a fragment thereof that has a common antigenic cross-reactivity with said HPV 16 L1 protein. Said fusion protein may: (a) be encoded by a polynucleotide which comprises or consists of a nucleic acid sequence having at least 90% identity with any one of SEQ ID NO: 3, 5, 6 or 8, 26, 27, 29, 30 or 32; and/or (b) comprise or consists of an amino acid sequence having at least 90% identity with any one of SEQ ID NO: 9, 10, 11, 12, 28, 31 or 33.

The one or more antigen derived from SARS-CoV-2 may be a VLP comprising a SARS-CoV-2 spike protein or immunogenic fragment thereof, wherein said VLP comprises or consists of a fusion protein of the invention.

The influenza HA or immunogenic fragment thereof and the influenza NA or immunogenic fragment thereof may be comprised in: (a) a seasonal influenza vaccine, in particular the seasonal 3-valent influenza vaccine or the seasonal 4-valent influenza vaccine; (b) a monovalent pandemic influenza vaccine; or (c) a universal influenza vaccine.

The invention also provides combined influenza-COVID-19 vaccine as described herein for use in a method of treatment and/or prevention of COVID-19 and influenza.

The invention further provides the use of an influenza HA or an immunogenic fragment thereof; and an antigen derived from SARS-CoV-2 or an immunogenic fragment thereof, and optionally an influenza NA or an immunogenic fragment thereof in the manufacture of a medicament for use in the treatment and/or prevention of COVID-19 and influenza, wherein said medicament is a combined influenza-COVID-19 vaccine as defined herein.

The invention further provides a method of immunising a subject against both influenza and COVID-19 comprising administering to said subject a therapeutically effective amount of a combined influenza-COVID-19 vaccine as defined herein.

The combined influenza-COVID-19 vaccine may be administered at intervals of 10 to 14 months, optionally wherein the combined influenza-COVID-19 vaccine is administered at intervals of about 12 months.

### DESCRIPTION OF FIGURES

FIG. 1: Schematic of the coronavirus's structure and the function of the structural proteins.

FIG. 2: SDS Page (left) and Western Blot (centre and right) of HBSAg-(EAAAK)₃-CoV-S using rabbit-anti CoV-S(1:250, centre) and mouse anti-HBSAg-(EAAAK)₃-RBD (1:1000, right)

FIG. 3: Graph showing anti-HBSAg-(EAAAK)₃-CoV-S IgG titre quantified by ELISA assay on mice sera immunized with HBSAg-(EAAAK)₃-CoV-S protein alone and in combination with influenza vaccine VAXIGRIP, formulated with two different adjuvants: Alu-280 and Addavax 14 days after immunization.

FIG. 4: A Graph showing anti-HBSAg-(EAAAK)₃-RBD IgG titre quantified by ELISA assay on mice sera immunized with HBSAg-(EAAAK)₃-RBD, formulated with two different adjuvants: Alu-280 and Addavax 14 days after immunization. B Comparison of anti-HBSAg-(EAAAK)₃-CoV-S IgG and anti-HBSAg-(EAAAK)₃-RBD IgG titre quantified by ELISA assay on mice sera immunized with HBSAg-(EAAAK)₃-CoV-S or HBSAg-(EAAAK)₃-RBD, formulated with two different adjuvants: Alu-280 and Addavax 14 days after immunization.

FIG. 5: Graph showing anti-HBSAg-(EAAAK)₃-CoV-S IgG titre quantified by ELISA assay on mice sera immunized with HBSAg-(EAAAK)₃-CoV-S protein alone and in combination with influenza vaccine VAXIGRIP, formulated with two different adjuvants: Alu-280 and Addavax 42 days after immunization.

FIG. 6: A Graph showing anti-HBSAg-(EAAAK)₃-RBD IgG titre quantified by ELISA assay on mice sera immunized with HBSAg-(EAAAK)₃-RBD, formulated with two different adjuvants: Alu-280 and Addavax 42 days after immunization. B Comparison of anti-HBSAg-(EAAAK)₃-CoV-S IgG and anti-HBSAg-(EAAAK)₃-RBD IgG titre quantified by ELISA assay on mice sera immunized with HBSAg-(EAAAK)₃-CoV-S (alone or in combination with influenza vaccine VAXIGRIP) or HBSAg-(EAAAK)₃-RBD, formulated with two different adjuvants: Alu-280 and Addavax 42 days after immunization.

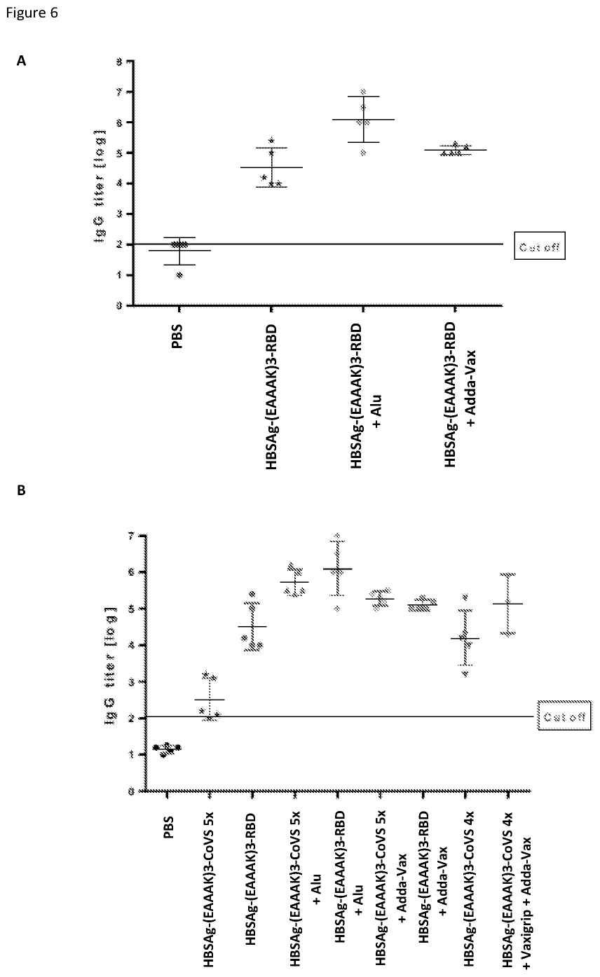

### DETAILED DESCRIPTION OF THE INVENTION

### Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure. The meaning and scope of the terms should be clear; however, in the event of any latent ambiguity, definitions provided herein take precedent over any dictionary or extrinsic definition. It should be understood that this invention is not limited to the particular methodology, protocols, and reagents, etc., described herein and as such can vary.

This disclosure is not limited by the exemplary methods and materials disclosed herein, and any methods and materials similar or equivalent to those described herein can be used in the practice or testing of embodiments of this disclosure. The terminology used herein is for the purpose of describing particular embodiments only, and is not intended to limit the scope of the present invention, which is defined solely by the claims.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

As used herein, the term “capable of” when used with a verb, encompasses or means the action of the corresponding verb. For example, “capable of interacting” also means interacting, “capable of cleaving” also means cleaves, “capable of binding” also means binds and “capable of specifically targeting . . . ” also means specifically targets.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

As used herein, the articles “a” and “an” may refer to one or to more than one (e.g. to at least one) of the grammatical object of the article. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (t) 5%, preferably ±4%, ±3%, ±2%, ±1%, ±0.5%, ±0.1%, of the numerical value of the number with which it is being used.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention.

Embodiments described herein as “comprising” one or more features may also be considered as disclosure of the corresponding embodiments “consisting of” and/or “consisting essentially of” such features.

The term “pharmaceutically acceptable” as used herein means approved by a regulatory agency of the Federal or a state government, or listed in the U.S. Pharmacopeia, European Pharmacopeia or other generally recognized pharmacopeia for use in animals, and more particularly in humans.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

The term “variant”, when used in relation to a protein, means a peptide or peptide fragment of the protein that contains one or more analogues of an amino acid (e.g. an unnatural amino acid), or a substituted linkage.

The term “derivative”, when used in relation to a protein, means a protein that comprises the protein in question, and a further peptide sequence. The further peptide sequence should preferably not interfere with the basic folding and thus conformational structure of the original protein. Two or more peptides (or fragments, or variants) may be joined together to form a derivative. Alternatively, a peptide (or fragment, or variant) may be joined to an unrelated molecule (e.g. a second, unrelated peptide). Derivatives may be chemically synthesized, but will be typically prepared by recombinant nucleic acid methods. Additional components such as lipid, and/or polysaccharide, and/or polypeptide components may be included.

As used herein, the terms “protein” and “polypeptide” are used interchangeably herein to designate a series of amino acid residues, connected to each other by peptide bonds between the alpha-amino and carboxyl groups of adjacent residues. The terms “protein”, and “polypeptide” refer to a polymer of amino acids, including modified amino acids (e.g., phosphorylated, glycated, glycosylated, etc.) and amino acid analogues, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

Proteins of the invention may include variants in which amino acid residues from one species are substituted for the corresponding residue in another species, either at the conserved or non-conserved positions. Variants of protein molecules disclosed herein may be produced and used in the present invention. Following the lead of computational chemistry in applying multivariate data analysis techniques to the structure/property-activity relationships [see for example, Wold, et al. Multivariate data analysis in chemistry. Chemometrics-Mathematics and Statistics in Chemistry (Ed.: B. Kowalski); D. Reidel Publishing Company, Dordrecht, Holland, 1984 (ISBN 90-277-1846-6] quantitative activity-property relationships of proteins can be derived using well-known mathematical techniques, such as statistical regression, pattern recognition and classification [see for example Norman et al. Applied Regression Analysis. Wiley-Interscience; 3rd edition (April 1998) ISBN: 0471170828; Kandel, Abraham et al. Computer-Assisted Reasoning in Cluster Analysis. Prentice Hall PTR, (May 11, 1995), ISBN: 0133418847; Krzanowski, Wojtek. Principles of Multivariate Analysis: A User's Perspective (Oxford Statistical Science Series, No 22 (Paper)). Oxford University Press; (December 2000), ISBN: 0198507089; Witten, Ian H. et al Data Mining: Practical Machine Learning Tools and Techniques with Java Implementations. Morgan Kaufmann; (Oct. 11, 1999), ISBN:1558605525; Denison David G. T. (Editor) et al Bayesian Methods for Nonlinear Classification and Regression (Wiley Series in Probability and Statistics). John Wiley & Sons; (July 2002), ISBN: 0471490369; Ghose, Arup K. et al. Combinatorial Library Design and Evaluation Principles, Software, Tools, and Applications in Drug Discovery. ISBN: 0-8247-0487-8]. The properties of proteins can be derived from empirical and theoretical models (for example, analysis of likely contact residues or calculated physicochemical property) of protein sequence, functional and three-dimensional structures and these properties can be considered individually and in combination.

Amino acids are referred to herein using the name of the amino acid, the three-letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Amino acid residues at non-conserved positions may be substituted with conservative or non-conservative residues. In particular, conservative amino acid replacements are contemplated.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in an antibody of the invention does not exclude other forms of variant, for example polymorphic variants, interspecies homologs, and alleles.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

Reference to SARS-CoV-2 polynucleotides and/or proteins in the present specification embraces fragments and variants thereof.

As used herein, the term “fragment” in the context of a SARS-CoV-2 spike protein refers to a part of the protein which may comprise one or more domain or part-domain of the full-length SARS-CoV-2 spike protein. A SARS-CoV-2 spike protein fragment according to the invention may typically be an immunogenic fragment as described herein. A fragment of a SARS-CoV-2 spike protein is typically greater than 200 amino acids in length. SARS-CoV-2 spike protein fragments of the present invention may comprise or consist of at least 200, at least 300, at least 400, at least 500, at least 600, at least 700, at least 800, at least 900, at least 1000, at least 1100, or more amino acid residues in length. The fragments of the invention typically have a common antigenic cross-reactivity with the SARS-CoV-2 spike protein (and so are referred to as immunogenic fragments). A SARS-CoV-2 spike protein fragment may comprise or consist of (i) a receptor-binding domain (RBD) of a SARS-CoV-2 spike protein; (ii) an N-terminal domain (NTD) of a SARS-CoV-2 spike protein; (iii) a C-terminal domain (CTD) of a SARS-CoV-2 spike protein, such as CTD1 and/or CTD2, these CTD are also known as subdomains (SD), with CTD1 also being known as SD1 and CTD2 also being known as SD2; and/or (iv) a fusion peptide (FP); and/or (v) FPPR domain; or any combination thereof. In particular, a fragment of a SARS-CoV-2 spike protein according to the invention may comprise or consist of an RBD domain. By way of non-limiting example, a fragment of a SARS-CoV-2 spike protein according to the invention may consist of an RBD domain, or may comprise an RBD domain in combination with an NTD domain.

Variant SARS-CoV-2 spike proteins retain one or more conformational epitope of native spike protein and the ability to elicit the production of neutralising antibodies and/or an immunoprotective response. Variant SARS-CoV-2 spike protein polynucleotides of the invention encode such spike proteins. By way of example, a variant may have at least 80%, preferably at least 90%, more preferably at least 95%, and most preferably at least 97% or at least 99% amino acid sequence homology with the reference sequence (e.g. a SARS-CoV-2 polynucleotide and/or protein of the invention, particularly any SEQ ID NO presented in the present specification which defines a SARS-CoV-2 polynucleotide and/or protein). Thus, a variant may include one or more analogues of a polynucleotide (e.g. an unnatural nucleic acid), or a substituted linkage. Also, by way of example, the term fragment, when used in relation to a SARS-CoV-2 polynucleotide and/or protein, means a polynucleotide having at least ten, preferably at least fifteen, more preferably at least twenty nucleic acid residues of the reference SARS-CoV-2 polynucleotide and/or protein. The term fragment also relates to the above-mentioned variants. Thus, by way of example, a fragment of a SARS-CoV-2 polynucleotide and/or protein of the present invention may comprise a nucleic acid sequence having at least 10, 20 or 30 nucleic acids, wherein the polynucleotide sequence has at least 80% sequence homology over a corresponding nucleic acid sequence (of contiguous) nucleic acids of the reference SARS-CoV-2 polynucleotide and/or protein sequence. These definitions of fragments and variants also apply to other polynucleotides of the invention. In the context of peptide sequences, the term fragment means a peptide having at least ten, preferably at least fifteen, more preferably at least twenty amino acid residues of the reference protein. The term fragment also relates to the above-mentioned variants. Thus, by way of example, a fragment may comprise an amino acid sequence having at least 10, 20 or 30 amino acids, wherein the amino acid sequence has at least 80% sequence homology over a corresponding amino acid sequence (of contiguous) amino acids of the reference sequence.

Preferably, the variant is a conservative substitution variant. A “variant,” as referred to herein, is a polypeptide substantially homologous to a native or reference polypeptide, but which has an amino acid sequence different from that of the native or reference polypeptide because of one or a plurality of deletions, insertions or substitutions. Polypeptide-encoding DNA sequences encompass sequences that comprise one or more additions, deletions, or substitutions of nucleotides when compared to a native or reference DNA sequence, but that encode a variant protein or fragment thereof that retains the relevant biological activity relative to the reference protein, e.g., at least 50% of the wildtype reference protein. As to amino acid sequences, one of skill will recognize that individual substitutions, deletions or additions to a nucleic acid, peptide, polypeptide, or protein sequence which alters a single amino acid or a small percentage, (i.e. 5% or fewer, e.g. 4% or fewer, or 3% or fewer, or 1% or fewer) of amino acids in the encoded sequence is a “conservatively modified variant” where the alteration results in the substitution of an amino acid with a chemically similar amino acid. It is contemplated that some changes can potentially improve the relevant activity, such that a variant, whether conservative or not, has more than 100% of the activity of wild-type, e.g. 110%, 125%, 150%, 175%, 200%, 500%, 1000% or more.

A polypeptide as described herein may comprise at least one peptide bond replacement. A single peptide bond or multiple peptide bonds, e.g. 2 bonds, 3 bonds, 4 bonds, 5 bonds, or 6 or more bonds, or all the peptide bonds can be replaced. An isolated peptide as described herein can comprise one type of peptide bond replacement or multiple types of peptide bond replacements, e.g. 2 types, 3 types, 4 types, 5 types, or more types of peptide bond replacements. Non-limiting examples of peptide bond replacements include urea, thiourea, carbamate, sulfonyl urea, trifluoroethylamine, ortho-(aminoalkyl)-phenylacetic acid, para-(aminoalkyl)-phenylacetic acid, meta-(aminoalkyl)-phenylacetic acid, thioamide, tetrazole, boronic ester, olefinic group, and derivatives thereof.

A polypeptide as described herein may comprise naturally occurring amino acids commonly found in polypeptides and/or proteins produced by living organisms, e.g. Ala (A), Val (V), Leu (L), Ile (I), Pro (P), Phe (F), Trp (W), Met (M), Gly (G), Ser (S), Thr (T), Cys (C), Tyr (Y), Asn (N), Gin (Q), Asp (D), Glu (E), Lys (K), Arg (R), and His (H). A polypeptide as described herein may comprise alternative amino acids.

Non-limiting examples of alternative amino acids include D amino acids, beta-amino acids, homocysteine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine (3-mercapto-D-valine), ornithine, citruline, alpha-methyl-alanine, para-benzoylphenylalanine, paraaminophenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, I-amino-1-cyclopentanecarboxylic acid, I-amino-1-cyclohexanecarboxylic acid, amino-benzoic acid, amino-naphthoic acid, gamma-aminobutyric acid, difluorophenylalanine, nipecotic acid, alphaamino butyric acid, thienyl-alanine, t-butylglycine, trifluorovaline; hexafluoroleucine; fluorinated analogs; azide-modified amino acids; alkyne-modified amino acids; cyano-modified amino acids; and derivatives thereof.

A polypeptide may be modified, e.g. by addition of a moiety to one or more of the amino acids comprising the peptide. A polypeptide as described herein may comprise one or more moiety molecules, e.g. 1 or more moiety molecules per peptide, 2 or more moiety molecules per peptide, 5 or more moiety molecules per peptide, 10 or more moiety molecules per peptide or more moiety molecules per peptide. A polypeptide as described herein may comprise one more types of modifications and/or moieties, e.g. 1 type of modification, 2 types of modifications, 3 types of modifications or more types of modifications. Non-limiting examples of modifications and/or moieties include PEGylation; glycosylation; HESylation; ELPylation; lipidation; acetylation; amidation; end-capping modifications; cyano groups; phosphorylation; albumin, and cyclization.

Alterations of the original amino acid sequence can be accomplished by any of a number of techniques known to one of skill in the art. Amino acid substitutions can be introduced, for example, at particular locations by synthesizing oligonucleotides containing a codon change in the nucleotide sequence encoding the amino acid to be changed, flanked by restriction sites permitting ligation to fragments of the original sequence. Following ligation, the resulting reconstructed sequence encodes an analogue having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations include those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. A polypeptide as described herein may be chemically synthesized and mutations can be incorporated as part of the chemical synthesis process.

As used herein, the terms “polynucleotides”, “nucleic acid” and “nucleic acid sequence” refers to any molecule, preferably a polymeric molecule, incorporating units of ribonucleic acid, deoxyribonucleic acid or an analogue thereof. The nucleic acid can be either single-stranded or double-stranded. A single-stranded nucleic acid can be one nucleic acid strand of a denatured double-stranded DNA Alternatively, it can be a single-stranded nucleic acid not derived from any double-stranded DNA. In one aspect, the nucleic acid can be DNA In another aspect, the nucleic acid can be RNA Suitable nucleic acid molecules are DNA, including genomic DNA or cDNA. Other suitable nucleic acid molecules are RNA, including mRNA.

A typical antibody comprises at least two “light chains” (LC) and two “heavy chains” (HC). The light chains and heavy chains of such antibodies are polypeptides consisting of several domains. Each heavy chain comprises a heavy chain variable region (abbreviated herein as “VH”) and a heavy chain constant region (abbreviated herein as “CH”). The heavy chain constant region comprises the heavy chain constant domains CH1, CH2 and CH3 (antibody classes IgA, IgD, and IgG) and optionally the heavy chain constant domain CH4 (antibody classes IgE and IgM). Each light chain comprises a light chain variable domain (abbreviated herein as “VL”) and a light chain constant domain (abbreviated herein as “CL”). The variable regions VH and VL can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The “constant domains” of the heavy chain and of the light chain are not involved directly in binding of an antibody to a target, but exhibit various effector functions.

Binding between an antibody and its target antigen or epitope is mediated by the Complementarity Determining Regions (CDRs). The CDRs are regions of high sequence variability, located within the variable region of the antibody heavy chain and light chain, where they form the antigen-binding site. The CDRs are the main determinants of antigen specificity. Typically, the antibody heavy chain and light chain each comprise three CDRs which are arranged non-consecutively. The antibody heavy and light chain CDR3 regions play a particularly important role in the binding specificity/affinity of the antibodies according to the invention and therefore provide a further aspect of the invention.

Thus, the term “antigen binding fragment” as used herein incudes any naturally-occurring or artificially-constructed configuration of an antigen-binding polypeptide comprising one, two or three light chain CDRs, and/or one, two or three heavy chain CDRs, wherein the polypeptide is capable of binding to the antigen.

The sequence of a CDR may be identified by reference to any number system known in the art, for example, the Kabat system (Kabat, E. A., et al., Sequences of Proteins of Immunological Interest, 5th ed., Public Health Service, National Institutes of Health, Bethesda, Md. (1991); the Chothia system (Chothia &, Lesk, “Canonical Structures for the Hypervariable Regions of Immunoglobulins,” J. Mol. Biol. 196, 901-917 (1987)); or the IMGT system (Lefranc et al., “IMGT Unique Numbering for Immunoglobulin and Cell Receptor Variable Domains and Ig superfamily V-like domains,” Dev. Comp. Immunol. 27, 55-77 (2003)).

For heavy chain constant region amino acid positions discussed in the invention, numbering is according to the EU index first described in Edelman, G. M., et al., Proc. Natl. Acad. Sci. USA 63 (1969) 78-85). The EU numbering of Edelman is also set forth in Kabat et al. (1991) (supra.). Thus, the terms “EU index as set forth in Kabat”, “EU Index”. “EU index of Kabat” or “EU numbering” in the context of the heavy chain refers to the residue numbering system based on the human IgG1 EU antibody of Edelman et al. as set forth in Kabat et al. (1991). The numbering system used for the light chain constant region amino acid sequence is similarly set forth in Kabat et al. (supra.). Thus, as used herein, “numbered according to Kabat” refers to the Kabat numbering system set forth in Kabat et al. (supra.).

The terms “decrease”, “reduced”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. The terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Preferably the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Preferably a subject is human. A subject can be male or female, adult or juvenile.

A subject can be one who has been previously diagnosed with or identified as suffering from or having a condition in need of treatment or one or more complications related to such a condition, and optionally, have already undergone treatment for a condition as defined herein or the one or more complications related to said condition. Alternatively, a subject can also be one who has not been previously diagnosed as having a condition as defined herein or one or more complications related to said condition. For example, a subject can be one who exhibits one or more risk factors for a condition or one or more complications related to said condition or a subject who does not exhibit risk factors.

A “subject in need” of treatment for a particular condition can be a subject having that condition, diagnosed as having that condition, or at risk of developing that condition.

References herein to the level of a particular molecule encompass the actual amount of the molecule, such as the mass, molar amount, concentration or molarity of the molecule. For example, in the context of the invention, references to the level of a particular molecule may refer to the concentration of the molecule.

The level of a molecule may be determined in any appropriate physiological compartment. Preferred physiological compartments include plasma, blood and/or serum. The level of a molecule may be determined from any appropriate sample from a patient, e.g. a plasma sample, a blood sample, a serum sample, a tissue sample, a bronchial-alveolar lavage (BAL) sample and/or a CSF sample. Other non-limiting examples of samples which may be tested are tissue or fluid samples urine and biopsy samples. Thus, by way of non-limiting example, the invention may reference the level (e.g. concentration) of a molecule in the plasma and/or BAL of a patient. The level of a molecule/biomarker pre-treatment with a binding member of the invention may be interchangeably referred to as the “baseline”.

The level of a molecule after treatment with a vaccine of the invention may be compared with the level of the molecule in the patient pre-treatment with the vaccine. The level of a molecule may be measured directly or indirectly, and may be determined using any appropriate technique. Suitable standard techniques are known in the art, for example Western blotting and enzyme-linked immunosorbent assays (ELISAs).

As used herein, the terms SARS-CoV-2 and 2019-nCoV are used interchangeably to refer to the viral pathogen which cases the disease COVID-19. Reference to a SARS-CoV-2 infection refers to the disease COVID-19. The terms COVID-19 vaccine (or vaccine against COVID-19) are also synonymous with the terms SARS-CoV-2 vaccine (or vaccine against SARS-CoV-2).

As used herein, the term “vaccine” is used to refer to a composition which induces an immune response. For example, the composition may induce an immune response in a patient to which it is administered.

A live attenuated vaccine comprises whole viral particles or virions which are capable of infecting and replicating in host cells, but have been modified in some way so that they do not cause disease.

A live vectored vaccine comprises a live viral vector, which is typically a non-pathogenic virus, that has been modified to express one or more antigen from the virus against which an immune response is to be raised. Typically the one or more antigen is a key antigen against which an immune response would be generated if a patient were exposed to the wild-type virus (i.e. is infected with the disease) or vaccinated with a live attenuated or inactivated vaccine. The antigen may be a protein antigen, or fragment thereof, or a polysaccharide antigen, or fragment thereof. The antigen may be expressed recombinantly or as a conjugate or fusion protein.

An inactivated vaccine comprises whole viral particles or virions which have been killed or inactivated (e.g. by heat or chemical treatment). Inactivated virions are not capable of infecting or replicating in host cells and do not cause disease.

A subunit vaccine comprises one or more component of the virus against which an immune response is to be raised. Typically the one or more component is a key antigen against which an immune response would be generated if a patient were exposed to the wild-type virus (i.e. is infected with the disease) or vaccinated with a live attenuated or inactivated vaccine. The component may be a protein antigen, or fragment thereof, or a polysaccharide antigen, or fragment thereof. The component may be expressed recombinantly or as a conjugate or fusion protein.

The publications discussed herein are provided solely for their disclosure prior to the filing date of the present application. Nothing herein is to be construed as an admission that such publications constitute prior art to the claims appended hereto.

### Combination Vaccines

A common complication when attempting to generate combined vaccine compositions is the phenomenon known as component suppression (also known as antigen composition). Component suppression describes the situation where two or more vaccines or vaccine antigens, typically from different pathogens, are administered at the same time and the immune response elicited by one or more of the vaccines or vaccine antigens is compromised compared with the immune response elicited when the vaccines or vaccine antigens are administered separately. The immune response can be compromised in several ways. For example, the immune response elicited by one or more of the vaccines or vaccine antigens may be reduced compared with the immune response elicited when the vaccines or vaccine antigens are administered separately. Seroconversion and/or seropositivity may also be reduced compared with seroconversion and/or seropositivity when the vaccines or vaccine antigens are administered separately. The phenomenon of component suppression has been observed in relation to vaccines against bacterial pathogens (e.g. for pertussis-diphtheria-tetanus (DTaP) vaccine and Haemophilus influenza b (Hib) vaccine) and for vaccines against viral pathogens (e.g. yellow fever vaccine and measles-mumps-rubella (MMR) vaccine. Component suppression has also been observed when vaccine antigens are administered in the same composition, and even when pre-existing effective vaccine compositions are administered at the same time. The risk of component suppression means it is not possible to predict whether a combination vaccine will be clinically efficacious or not, or even whether two separate vaccine compositions maybe administered together. The risk of component suppression is commonly understood in the field of immunology, and is factored into considerations of vaccine scheduling and assessment of component suppression is a requirement by medical regulatory authorities.

The present inventors have demonstrated for the first time that it is possible to administer a vaccine comprising both influenza antigens and an antigen derived from SARS-CoV-2 and achieve good immunogenicity against both influenza and SARS-CoV-2, i.e. that component suppression does not occur in the context of influenza and SARS-CoV-2.

Accordingly, the present invention provides a combined influenza-COVID-19 vaccine (also referred to interchangeably herein as a combination influenza-COVID-19 vaccine) comprising: (a) an influenza haemagglutinin (HA) or an immunogenic fragment thereof; and (b) one or more antigen derived from SARS-CoV-2 or an immunogenic fragment thereof; wherein the antigens are capable of eliciting immune response and protection against both influenza and COVID-19 (as described herein). Typically said combined influenza-COVID-19 vaccine further comprises an influenza neuraminidase (NA) or an immunogenic fragment thereof.

As described herein, a combined influenza-COVID-19 vaccine of the invention is not associated with component suppression, or has minimal component suppression for: (i) the influenza HA or an immunogenic fragment thereof; (ii) the one or more antigen derived from SARS-CoV-2 (e.g. a SARS-CoV-2 spike protein) or an immunogenic fragment thereof; (iii) the optional influenza NA or immunogenic fragment thereof; or any combination thereof. Preferably a combined influenza-COVID-19 vaccine of the invention is not associated with component suppression, or has minimal component suppression for each of: (i) the influenza HA or an immunogenic fragment thereof; (ii) the one or more antigen derived from SARS-CoV-2 (e.g. a SARS-CoV-2 spike protein) or an immunogenic fragment thereof; and (iii) the optional influenza NA or an immunogenic fragment thereof; and.

As used herein, the term “not associated with component suppression” means that the immune response to (i) the influenza HA or an immunogenic fragment thereof; (ii) the one or more antigen derived from SARS-CoV-2 (e.g. a SARS-CoV-2 spike protein) or an immunogenic fragment thereof; (iii) the optional influenza NA or an immunogenic fragment thereof; or any combination thereof administered as part of a combined influenza-COVID-19 vaccine of the invention elicits essentially the same immune response as is achieved when the (i) the influenza HA or an immunogenic fragment thereof; (ii) the antigen derived from SARS-CoV-2 (e.g. a SARS-CoV-2 spike protein) or an immunogenic fragment thereof; and/or (iii) the optional influenza NA or an immunogenic fragment thereof; is administered separately.

As used herein, the term “has minimal component suppression” means that the immune response to (i) the influenza HA or an immunogenic fragment thereof; (ii) the one or more antigen derived from SARS-CoV-2 (e.g. a SARS-CoV-2 spike protein) or an immunogenic fragment thereof; (iii) the optional influenza NA or an immunogenic fragment thereof; or any combination thereof administered as part of a combined influenza-COVID-19 vaccine of the invention elicits at least 80%, at least 85%, at least 90%, at least 95%, at least 96%, at least 97%, at least 98%, at least 99% or more of the immune response as is achieved when the (i) the influenza HA or an immunogenic fragment thereof; (ii) the one or more antigen derived from SARS-CoV-2 (e.g. a SARS-CoV-2 spike protein) or an immunogenic fragment thereof; and/or (iii) the optional influenza NA or an immunogenic fragment thereof; is administered separately.

Another advantage of the combined influenza-COVID-19 vaccine of the invention is that patient compliance can be increased. The combined influenza-COVID-19 vaccines of the invention allow a patient to receive a single vaccine administration which will provide immunity to both influenza and SARS-CoV-2 infection. Reducing the number of vaccinations required and the number of clinic visits requires will increase vaccine uptake and patient compliance. In addition, many countries have well-established public health procedures and schedules for annual influenza vaccination programs. The combined influenza-COVID-19 vaccines of the invention allow for the coordinated wide-scale vaccination against SARS-CoV-2 infection making use of these existing programs and procedures, which will also facilitate wide-scale vaccination against SARS-CoV-2 infection without the need for new public health programs or infrastructure. In addition, some evidence suggests a potential association of climate and seasonality with COVID-19 infection and spread. The invention therefore has the potential to allow for regular (e.g. seasonal or annual) vaccination against COVID-19 as described herein, and hence to mitigate seasonal infection and spread. Furthermore, this can potentially be achieved by facilitating COVID-19 vaccination using the existing public health programs and procedures, particularly those already in place for seasonal influenza vaccination.

The influenza HA or immunogenic fragment thereof and the optional influenza NA or immunogenic fragment thereof may each be readily selected by a skilled person using routine skill. Non-limiting examples of influenza HA (or immunogenic fragments thereof) and influenza NA (or immunogenic fragments thereof) are described herein.

The one or more SARS-CoV-2 antigen or immunogenic fragment thereof may be readily selected by a skilled person using routine skill. Non-limiting examples of SARS-CoV-2 antigens (or immunogenic fragments thereof) are described herein. Typically the one or more SARS-CoV-2 antigen comprises at least one SARS-CoV-2 antigen spike protein or immunogenic fragment thereof, as described herein.

The influenza HA or immunogenic fragment thereof and/or the optional influenza NA or immunogenic fragment thereof may be comprised in an existing influenza vaccine composition. Said influenza vaccine composition may be combined with one or more SARS-CoV-2 antigen (e.g. at least one SARS-CoV-2 spike protein) or an immunogenic fragment thereof, or an existing COVID-19 vaccine to produce a combined influenza-COVID-19 vaccine according to the invention.

The one or more antigen derived from SARS-CoV-2 (e.g. at least one SARS-CoV-2 spike protein) or an immunogenic fragment thereof may be comprised in an existing COVID-19 vaccine composition. Said COVID-19 vaccine composition may be combined with an influenza HA or immunogenic fragment thereof and/or the optional influenza NA or immunogenic fragment thereof, or an existing influenza vaccine to produce a combined influenza-COVID-19 vaccine according to the invention. Typically when a live (attenuated or vectored) COVID-19 vaccine is used, a live (attenuated or vectored) influenza vaccine is used. Typically when an inactivated or subunit COVID-19 vaccine is used, an inactivated or subunit influenza vaccine is used. Preferably a subunit (including fusion protein and VLPs as described herein) COVID-19 vaccine or component is used and an inactivated influenza vaccine is used.

Accordingly, the influenza HA or immunogenic fragment thereof comprised in a combined influenza-COVID-19 vaccine of the invention may be: (i) comprised in an inactivated influenza virion; (ii) a recombinant HA or immunogenic fragment thereof; (iii) a fusion protein comprising HA or an immunogenic fragment thereof; or (iv) encoded by an RNA or DNA vaccine. Non-limiting examples of influenza HA, immunogenic fragments thereof, and influenza vaccines comprising HA are described herein.

The (optional) influenza NA or immunogenic fragment thereof comprised in a combined influenza-COVID-19 vaccine of the invention may be: (i) comprised in an inactivated influenza virion; (ii) a recombinant NA or immunogenic fragment thereof; (iii) a fusion protein comprising NA or an immunogenic fragment thereof; or (iv) encoded by an RNA or DNA vaccine. Non-limiting examples of influenza NA, immunogenic fragments thereof, and influenza vaccines comprising NA are described herein.

The one or more antigen derived from SARS-CoV-2 or an immunogenic fragment thereof comprised in a combined influenza-COVID-19 vaccine of the invention is preferably: (i) at least one recombinant SARS-CoV-2 spike protein or immunogenic fragment thereof; (ii) at least one fusion protein comprising a SARS-CoV-2 spike protein or immunogenic fragment thereof; (iii) at least one virus-like particle (VLP) comprising a SARS-CoV-2 spike protein or immunogenic fragment thereof; (iv) at least one polynucleotide encoding a recombinant SARS-CoV-2 spike protein or immunogenic fragment thereof; or (v) encoded by an RNA or DNA vaccine. Non-limiting examples of such SARS-CoV-2 antigens, particularly SARS-CoV-2 spike proteins, and immunogenic fragments thereof, and COVID-19 vaccines are described herein.

Any combination of (i) influenza HA, immunogenic fragments thereof, and influenza vaccines comprising HA; (ii) one or more SARS-CoV-2 antigens, particularly SARS-CoV-2 spike proteins, and immunogenic fragments thereof, and COVID-19 vaccines; and optionally (iii) influenza NA, immunogenic fragments thereof, and influenza vaccines comprising NA; may be used in a combined influenza-COVID-19 vaccine according to the present invention, provided that the HA, (optional) NA and SARS-CoV-2 antigens are capable of eliciting immune response and protection against both influenza and COVID-19.

The influenza component of a combined influenza-COVID-19 vaccine of the present invention may comprise a live (attenuated or vectored) influenza vaccine, an inactivated influenza vaccine or a subunit influenza vaccine.

Non-limiting examples of live attenuated influenza vaccines include: seasonal influenza vaccines, such as seasonal quadrivalent (4-valent) influenza vaccine. By way of specific non-limiting example, a seasonal quadrivalent influenza vaccine (e.g. the 2019-2020 season) may comprise an attenuated influenza A H1N1 virus, attenuated influenza A H3N2 virus and two influenza B viruses (B/Colorado/06/2017-like (Victoria lineage) virus and B/Phuket/3073/2013-like virus (Yamagata lineage)).

Non-limiting examples of inactivated influenza vaccines include: seasonal influenza vaccines, such as seasonal trivalent (3-valent) influenza vaccine and seasonal quadrivalent (4-valent) influenza vaccine. By way of specific non-limiting example, a seasonal trivalent influenza vaccine (e.g. the 2019-2020 season) may comprise an attenuated influenza A H1N1 virus, attenuated influenza A H3N2 virus and an influenza B virus (B/Colorado/06/2017-like (Victoria lineage)). By way of a further specific non-limiting example, a seasonal quadrivalent influenza vaccine (e.g. the 2019-2020 season) may comprise an attenuated influenza A H1N1 virus, attenuated influenza A H3N2 virus and two influenza B viruses (B/Colorado/06/2017-like (Victoria lineage) virus and B/Phuket/3073/2013-like virus (Yamagata lineage)).

Other examples of influenza vaccines that may be used in the combined influenza-COVID-19 vaccines of the invention include monovalent pandemic influenza vaccines (current pandemic influenza vaccines preapproved by the EMA include live attenuated or inactivated vaccines) and universal influenza vaccine (examples under development include subunit vaccines and two-stage vaccines comprising a priming DNA vaccine and a live vectored vaccine).

Preferably the influenza component of a combined influenza-COVID-19 vaccine of the present invention is a live attenuated or inactivated influenza vaccine.

The SARS-CoV-2 component of a combined influenza-COVID-19 vaccine of the present invention may comprise a live (attenuated or vectored) SARS-CoV-2/COVID-19 vaccine, an inactivated SARS-CoV-2/COVID-19 vaccine or a subunit SARS-CoV-2/COVID-19 vaccine.

Preferably the SARS-CoV-2 component of a combined influenza-COVID-19 vaccine of the present invention is a subunit vaccine comprising a SARS-CoV-2 spike protein or fragment thereof, or a fusion protein or VLP comprising said SARS-CoV-2 spike protein or fragment thereof.

Particularly preferred are combined influenza-COVID-19 vaccines in which the influenza component is a live attenuated or inactivated influenza vaccine and the SARS-CoV-2 component is a subunit vaccine comprising a SARS-CoV-2 spike protein or fragment thereof, or a fusion protein or VLP comprising said SARS-CoV-2 spike protein or fragment thereof.

Typically when the influenza component of a combined influenza-COVID-19 vaccine of the present invention comprises a live (attenuated or vectored) influenza vaccine, the SARS-CoV-2 component comprises a live (attenuated or vectored) SARS-CoV-2/COVID-19 vaccine.

Typically when the influenza component of a combined influenza-COVID-19 vaccine of the present invention comprises an inactivated influenza vaccine, the SARS-CoV-2 component comprises an inactivated SARS-CoV-2/COVID-19 vaccine. Alternatively, when the influenza component of a combined influenza-COVID-19 vaccine of the present invention comprises an inactivated influenza vaccine, the SARS-CoV-2 component comprises a subunit SARS-CoV-2/COVID-19 vaccine, or vice versa.

Typically when the influenza component of a combined influenza-COVID-19 vaccine of the present invention comprises a subunit influenza vaccine, the SARS-CoV-2 component comprises a subunit SARS-CoV-2/COVID-19 vaccine. Alternatively, when the influenza component of a combined influenza-COVID-19 vaccine of the present invention comprises a subunit influenza vaccine, the SARS-CoV-2 component comprises an inactivated SARS-CoV-2/COVID-19 vaccine, or vice versa.

Typically when the influenza component of a combined influenza-COVID-19 vaccine of the present invention comprises a nucleic acid (DNA or RNA, preferably DNA) influenza vaccine, the SARS-CoV-2 component comprises a nucleic acid (DNA or RNA, preferably DNA) SARS-CoV-2/COVID-19 vaccine.

The invention provides a combined influenza-COVID-19 vaccine wherein the influenza HA or immunogenic fragment thereof and the influenza NA or immunogenic fragment thereof are comprised in an inactivated influenza virion, and the one or more antigen derived from SARS-CoV-2 (e.g. at least one SARS-CoV-2 spike protein) or an immunogenic fragment thereof is: (i) at least one fusion protein comprising a SARS-CoV-2 spike protein or immunogenic fragment thereof; (ii) at least one virus-like particle (VLP) comprising a SARS-CoV-2 spike protein or immunogenic fragment thereof; or an inactivated SARS-CoV-2 virion.

The invention provides a combined influenza-COVID-19 vaccine wherein the influenza HA or immunogenic fragment thereof and optionally the influenza NA or immunogenic fragment thereof are comprised in a subunit vaccine, and the one or more antigen derived from SARS-CoV-2 (e.g. at least one SARS-CoV-2 spike protein) or an immunogenic fragment thereof is: (i) at least one fusion protein comprising a SARS-CoV-2 spike protein or immunogenic fragment thereof; (ii) at least one virus-like particle (VLP) comprising a SARS-CoV-2 spike protein or immunogenic fragment thereof; or an inactivated SARS-CoV-2 virion.

The invention provides a combined influenza-COVID-19 vaccine, wherein: the influenza HA or immunogenic fragment thereof is comprised in a live attenuated influenza virion; the influenza NA or immunogenic fragment thereof is comprised in a live attenuated influenza virion; and/or the one or more antigen derived from SARS-CoV-2 or an immunogenic fragment thereof is comprised in a live viral vector (i.e. in a live vectored vaccine). The live viral vector comprising the one or more antigen derived from SARS-CoV-2 or an immunogenic fragment thereof may be any viral vector used clinically for vaccines. Non-limiting examples include adenoviral vectors, measles virus vectors, mumps virus vectors, rubella virus vectors, varicella virus vectors, polio virus vectors and yellow fever virus vectors.

### Coronavirus Antigens

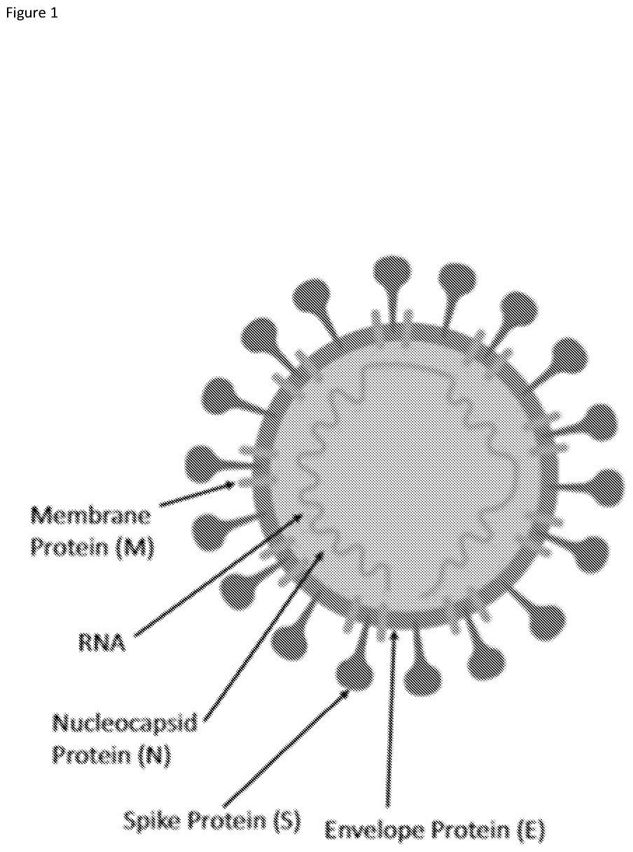

Coronaviruses (CoVs) have the largest genome among all RNA viruses, typically ranging from 27 to 32 kb. The CoV genome codes for at least four main structural proteins: spike (S), membrane (M), envelope (E), nucleocapsid (N) proteins and other accessory proteins which aid the replicative processes and facilitate entry into cells. FIG. 1 summarises the coronavirus's structure and the function of the structural proteins. Briefly, the CoV genome is packed inside a helical capsid formed by the nucleocapsid and further surrounded by an envelope. Associated with the viral envelope are at least three structural proteins: the membrane and envelope proteins, which are involved in virus assembly, and the spike protein, which mediates virus entry into host cells. Some coronaviruses also encode an envelope-associated hemagglutinin-esterase protein (HE). The spike protein forms large protrusions from the virus surface, giving coronaviruses the appearance of having crowns, from which the name “Coronavirus” is derived. As well as mediating virus entry, the spike protein is a critical determinant of viral host range and tissue tropism and a major inducer of host immune responses.

2019-nCoV (officially named severe acute respiratory syndrome coronavirus 2, SARS-CoV-2) is the causative agent of coronavirus disease 2019 (COVID-19) and is contagious among humans. It is believed that SARS-CoV-2 originated in animals, with bats being a likely source given the genetic similarities of SARS-CoV-2 to SARS-CoV (79.5%) and bat coronaviruses (96%). Any disclosure herein in relation to CoVs also applies directly and without restriction to SARS-CoV-2.

The one or more antigen derived from SARS-CoV-2 or an immunogenic fragment thereof in a combined influenza-COVID-19 vaccine of the invention maybe any SARS-CoV-2 antigen(s) which is capable of eliciting immune response and/or protection against SARS-CoV-2 infection. Preferably said one more antigen is: (i) at least one recombinant SARS-CoV-2 spike protein or immunogenic fragment thereof; (ii) at least one fusion protein comprising a SARS-CoV-2 spike protein or immunogenic fragment thereof; (iii) at least one virus-like particle (VLP) comprising a SARS-CoV-2 spike protein or immunogenic fragment thereof; (iv) at least one polynucleotide encoding a recombinant SARS-CoV-2 spike protein or immunogenic fragment thereof; or (v) encoded by at least one RNA or DNA vaccine.

The SARS-CoV-2 component of the combined influenza-COVID-19 vaccine of the invention may comprise at least one, at least two, at least three, at least four, or more SARS-CoV-2 antigens. By way of non-limiting example, each SARS-CoV-2 antigen may be a different spike protein antigen, such as the wild-types spike protein antigen and/or one of the variant spike proteins described herein. Other non-limiting examples of SARS-CoV-2 antigens that may be included in a combined influenza-COVID-19 vaccine of the present invention include such antigens from the 2019-CoV capsid, membrane protein or envelope protein. Each of the one or more SARS-CoV-2 antigens may be independently provided in the form of (i) a recombinant antigen or immunogenic fragment thereof; (ii) a fusion protein or immunogenic fragment thereof; (iii) a virus-like particle (VLP) comprising said antigen or immunogenic fragment thereof; or (iv) a polynucleotide encoding said antigen or immunogenic fragment thereof. The disclosure herein in relation to recombinant, fusion protein, VLP, polynucleotide and vectors comprising SARS-CoV-2 spike protein antigens is equally applicable to other SARS-CoV-2 antigens that may be comprised in a combined influenza-COVID-19 vaccine of the invention.

### Spike Protein

The CoV spike protein comprises three domains: (i) a large ectodomain; (ii) a transmembrane domain (which passes through the viral envelope in a single pass); and (iii) a short intracellular tail. The ectodomain consists of three receptor-binding subunits (3×S1) and a trimeric stalk made of three membrane-fusion subunits (3×S2). Thus, the SARS-CoV-2 spike protein is a homotrimer. During virus entry, S1 binds to a receptor on the host cell surface for viral attachment, and S2 fuses the host and viral membranes, allowing viral genomes to enter host cells. Receptor binding and membrane fusion are the initial and critical steps in the coronavirus infection cycle. There is significant divergence in the receptors targeted by different CoVs.

The structure of the SARS-CoV-2 spike protein is described, for example, in Cai et al. (Science (2020) 369:1586-1592)), which is herein incorporated by reference in its entirety. Each S1 subunit of a SARS-CoV-2 spike protein comprises an N-terminal domain (NTD), receptor binding domain (RBD), two C-terminal domains (CTDs). Prior to fusion with the host cell membrane, the S1 subunits of the SARS-CoV-2 spike protein protect the S2 subunits. On binding to ACE2, the SARS-CoV-2 spike protein refolds in a “jack-knife” manner, forming a long-central coiled coil and ultimately leading to membrane fusion and viral entry to a host cell.

The present inventors have previously shown that the SARS-CoV-2 spike protein and immunogenic fragments thereof have therapeutic potential (including prophylactic potential) as antigens for vaccines against SARS-CoV-2/COVID-19 infection.

Accordingly, as described herein, the one or more antigen derives from SARS-CoV-2 contained in a combined influenza-COVID-19 vaccine of the invention is preferably one or more SARS-CoV-2 spike protein or immunogenic fragment thereof. Typically said one or more SARS-CoV-2 spike protein has at least 70%, at least 75%, at least 80%, at least 90%, at least 95%, at least 96%, at least 97%, at least 98%, at least 99% or more identity with SEQ ID NO: 1, or a fragment thereof, that has a common antigenic cross-reactivity with said spike protein. Preferably the one or more spike protein from SARS-CoV-2 has at least 90%, at least 95%, at least 96%, at least 97%, at least 98%, at least 99% or more identity with SEQ ID NO: 1, or a fragment thereof, that has a common antigenic cross-reactivity with said spike protein. More preferably, the one or more spike protein from SARS-CoV-2 has at least 98%, at least 99% or more with SEQ ID NO: 1, or a fragment thereof, that has a common antigenic cross-reactivity with said spike protein. The one or more spike protein from SARS-CoV-2 may comprise or consist of SEQ ID NO: 1, or a fragment thereof, that has a common antigenic cross-reactivity with said spike protein (also referred to herein as an immunogenic fragment).

A SARS-CoV-2 spike protein or immunogenic fragment thereof according to the invention typically retain the same binding affinity for its receptor as the native SARS-CoV-2 spike protein. In the context of the present invention, this may mean having a binding affinity for the SARS-CoV-2 spike protein receptor of at least 80%, at least 85%, at least 90%, at least 95%, at least 99% or more of that of the native SARS-CoV-2 spike protein. Preferably the SARS-CoV-2 spike protein or immunogenic fragment thereof of the invention have a binding affinity for the SARS-CoV-2 spike protein of at least 90%, at least 95%, at least 99% or more of that of the native SARS-CoV-2 spike protein.

In some embodiments, the SARS-CoV-2spike protein or immunogenic fragment thereof of the invention have a binding affinity for the 2019-nCoV spike protein receptor greater than that of the full-length protein. For example, the SARS-CoV-2 spike protein or immunogenic fragment thereof of the invention of the invention may have a binding affinity of at least 100%, at least 110%, at least 120%, or at least 150% or more of that of the native SARS-CoV-2 spike protein.

In other embodiments, the SARS-CoV-2 spike protein or immunogenic fragment thereof of the invention may have a binding affinity for the SARS-CoV-2 spike protein receptor less than that of the native SARS-CoV-2 spike protein. For example, the SARS-CoV-2 spike protein or immunogenic fragment thereof of the invention may have a binding affinity of less than 80%, less than 70%, less than 60%, less than 50% or less of that of the native SARS-CoV-2 spike protein.

The binding affinity of a SARS-CoV-2 spike protein or immunogenic fragment thereof expressed by a polynucleotide of the invention for its receptor may be quantified in terms of dissociation constant (K_{d}). K_{d} may be determined using any appropriate technique, but surface plasmon resonance (SPR) is generally preferred in the context of the present invention.

An immunogenic fragment of the one or more SARS-CoV-2 spike protein is typically greater than 200 amino acids in length. SARS-CoV-2 spike protein fragments of the present invention may comprise or consist of at least 200, at least 300, at least 400, at least 500, at least 600, at least 700, at least 800, at least 900, at least 1000, at least 1100, or more amino acid residues in length. The fragments of the invention have a common antigenic cross-reactivity with the SARS-CoV-2 spike protein (and so are referred to as immunogenic fragments).

According to the present invention, the one or more SARS-CoV-2 spike protein or fragment thereof maintains one or more conformational epitope present in native (wild-type) SARS-CoV-2 spike protein. As such, the one or more SARS-CoV-2 spike protein or fragment thereof is capable of giving rise to an immunoprotective effect. Typically said immunoprotective effect comprises the production of neutralising antibodies (nAb) which specifically bind to the one or more conformational epitope of the SARS-CoV-2 spike protein or fragment thereof. A conformational epitope of a CoV spike protein has a specific three-dimensional structure that is found in the tertiary structure of the CoV spike protein. Said one or more conformational epitope is typically within the ectodomain of the spike protein. Preferably the one or more SARS-CoV-2 spike protein or fragment thereof retains all of the conformational epitopes present in native SARS-CoV-2 spike protein.

An immunogenic fragment of a SARS-CoV-2 protein may comprise or consist of the RBD, NTD, CTD1, CDT2, FP, and/or FPPR, or any combination thereof. Preferably, the immunogenic fragment of SARS-CoV-2 spike protein comprises or consists of the receptor-binding domain (RBD) of the SARS-CoV-2 spike protein. This RBD is responsible for SARS-CoV-2 binding to a host cell and thus facilitates entry of SARS-CoV-2 particles into the host cell. The RBD corresponds to amino acid residues 319 to 529 of SEQ ID NO: 1, as described herein is referred to as SEQ ID NO: 15. The RBD is encoded by bases corresponding to positions 955 to 1597 in the genome of the SARS-CoV-2 virus (Genbank Accession No. MN908947, version 3 of which (MN908947.3) was deposited 17 Jan. 2020). Accordingly, as described herein, the invention relates to an RBD of the SARS-CoV-2 spike protein has at least 70%, at least 75%, at least 80%, at least 90%, at least 95%, at least 96%, at least 97%, at least 98%, at least 99% or more identity with SEQ ID NO: 15. Preferably the immunogenic fragment of SARS-CoV-2 spike protein comprises or consists of an RBD of the SARS-CoV-2 spike protein that has at least 90%, at least 95%, at least 96%, at least 97%, at least 98%, at least 99% or more identity with SEQ ID NO: 15. More preferably, the immunogenic fragment of SARS-CoV-2 spike protein comprises or consists of an RBD of the SARS-CoV-2 spike protein having least 98%, at least 99% or more with SEQ ID NO: 15. The RBD of the SARS-CoV-2 spike protein may comprise or consist of SEQ ID NO: 15. Any and all disclosure herein relating to the SARS-CoV-2 spike protein (e.g. in relation to polynucleotides, viral vectors, DNA plasmids, RNA vaccines, virus-like particles (VLPs), fusion proteins, antibodies, compositions and pharmaceutical compositions, formulations and therapeutic indications) applies equally and without reservation to the RBD of the SARS-CoV-2 spike protein.

CoVs are large enveloped single positive-sense RNA viruses. Mutation rates of RNA viruses are greater than DNA viruses, suggesting a more efficient adaptation process for survival. Thus, there is a risk that antigenic drift, similar to that observed for influenza virus, will also become a feature of the SARS-CoV-2, or is SARS-CoV-2 becomes endemic in the population once the pandemic has subsided. Indeed, research to-date has already identified mutations within the receptor binding domain (RBD) of the spike protein of SARS-CoV-2, particularly G476S and V483A/G, as well as a prevalent D614G mutation in the vicinity of the S1/S2 site (Saha et al., ChemRxiv™ http://doi.org/10.26434/chemrxiv.12320567.v1), which the evidence suggests can enhance cell entry by the SARS-CoV-2 virion, and also broaden the host cell tropism. Other mutations reported in the SARS-CoV-2 spike protein include 5943 (particularly S943P), L5 (particularly L5F), L8 (particularly L8F), V367 (particularly V367F), H49 (particularly H49Y), Y145 (particularly Y145H/del), Q239 (particularly Q239K), A831 (particularly A831V), D839 (particularly D839Y/N/E), and P1263 (particularly P1263L), or any combination thereof (Korber et al., BioRxiv™ https://doi.org/10.1101/2020.04.29.069054).

Accordingly, the invention advantageously allow SARS-CoV-2 vaccine antigens to be modified if required to provide enhanced immunity against strains with mutated spike proteins as they arise. By way of non-limiting example, any SARS-CoV-2 spike protein or fragment thereof according to the invention may be modified (particularly by substitution) at position (i) D614, (ii) V483, (iii) G476, (iv) K417, (v), E484, (vi) N501, (vii) A570, and (viii) P681, or any combination of (including any two, any three, any four, any five, any six, any seven or all eight) of (i) to (viii). Alternatively or in addition, the SARS-CoV-2 spike proteins or fragments thereof may comprise deletion mutations, including deletions at one or more of amino acid residues 69, 70 and/or 144. As described herein, the positions of the mutations/modifications typically corresponds to the numbering of amino acids in SEQ ID NO: 1 of the present invention.

Modification at position D614, particularly the D614G substitution, is preferred. In particular, any SARS-CoV-2 spike protein or fragment thereof according to the invention may comprise the following substitutions (i) G476S, (ii) V483A/G, (iii) D614G, (iv) K417N/T, (v), E484K, (vi) N501Y, (vii) A570D, and (viii) P681H, or any combination of (including any two, any three, any four, any five, any six, any seven or all eight) of (i) to (viii).

The invention also relates to SARS-CoV-2 spike proteins or fragments thereof from a variant SARS-CoV-2. In particular, the invention may relate to SARS-CoV-2 spike proteins or fragments thereof from the B.1.1.7 strain (also known as 201/501Y.V1, which was first detected in the UK, now known as the Alpha variant); the B.1.351 strain (also known as 20H/501.V2, which was first detected in South Africa, now known as the Beta variant), the P1 strain (also known as 20J/501Y.V3, which was first detected in Japan and Brazil, now known as the Gamma variant), the B1.427 and B1.429 strains (first detected in California, now known as the Epsilon variant), and/or the B.1.617.2 strain (which was first detected in India, now known as the Delta variant). According to the CDC (SARS-CoV-2 Variant Classifications and Definitions (cdc.gov)), the Alpha variant has been found to comprise the following mutations: 69deletion, 70deletion, 144deletion, (E484K*), (S494P*), N501Y, A570D, D614G, P681H, T7161, S982A, D1118H, and (K1191N*) The key mutations of the Alpha variant comprise deletion of residues 69/70 and 144Y, as well as N501Y, A570D, D614G and P681H substitutions. According to the CDC (SARS-CoV-2 Variant Classifications and Definitions (cdc.gov)), the Beta variant has been found to comprise the following mutations: D80A, D215G, 241deletion, 242deletion, 243deletion, K417N, E484K, N501Y, D614G, and A701V. The key mutations of the Beta variant comprise K417N, E484K, N501Y and D614G substitutions. According to the CDC SARS-CoV-2 Variant Classifications and Definitions (cdc.gov, the Gamma variant has been found to comprise the following mutations: L18F, T20N, P26S, D138Y, R190S, K417T, E484K, N501Y, D614G, H655Y, T10271. The key mutations of the Gamma variant comprise E484K, K417N/T, N501Y and D614G. According to the CDC (SARS-CoV-2 Variant Classifications and Definitions cdc.gov), the Delta variant has been found to comprise the following mutations: T19R, (G142D*), 156deletion, 157deletion, R158G, L452R, T478K, D614G, P681R, and D950N. The key mutations of the Delta variant comprise L452R, E484Q and T478K. According to the CDC (SARS-CoV-2 Variant Classifications and Definitions (cdc.gov)), the Epsilon variant has been found to comprise the following mutations: S131, W152C, L452R, D614G. The key mutation of the Epsilon variant is L452R.

All the disclosure herein in relation to combination vaccines, polynucleotides, spike proteins and fragments thereof, VLPs, fusion proteins and DNA/RNA vaccine applies equally to different variants and strains of SARS-CoV-2 unless explicitly stated.

Development of a vaccine composition which can be safely administered repeatedly would therefore not only enable boosting of the immune response to address issues of protective immunity being lost over time (as described herein and as observed in the clinic), but would also advantageously allow SARS-CoV-2 vaccine antigens to be modified if required to provide enhanced immunity against strains with mutated spike proteins as they arise. By way of non-limiting example, any SARS-CoV-2 spike protein or fragment thereof used as one or more SARS-CoV-2 antigen according to the invention may be modified (particularly by substitution) at position: (i) 417; (ii) 452; (iii) 478; (iv) 484; (v) 201; (vi) 570; (vii) 614; and/or (viii) 681; or any combination thereof. By way of further non-limiting example, any SARS-CoV-2 spike protein or fragment thereof used as one or more SARS-CoV-2 antigen according to the invention may be modified (particularly by substitution) at position (i) D614, (ii) V483, (iii) G476, (iv) G476 and V483, (v) G476 and D614, (vi) V483 and D614, or (vii) G476, V483 and D614. Modification at position D614, particularly the D614G substitution, may be preferred. Modification at position L452, particularly the L452R substitution, may be preferred. In particular, any SARS-CoV-2 spike protein or fragment thereof used as the one or more SARS-CoV-2 antigen according to the invention may comprise the following substitutions (i) G476S, (ii) V483A/G, (iii) D614G, (iv) G476S and V483A/G, (v) G476S and D614G, (vi) V483A/G and D614G, (vii) G476S, V483A/G and D614G, (viii) L452R and E484Q, and optionally T478K; or (ix) L452R. Multiple variant SARS-CoV-2 spike proteins (in any of the forms described herein, particularly as fusion proteins or VLPs) may be comprised in a combined influenza-COVID-19 vaccine of the invention.

### Polynucleotides

The one or more antigen derived from SARS-CoV-2 or an immunogenic fragment thereof may be encoded or expressed by one or more polynucleotide vaccine (the terms “encode” and “express” are used interchangeably herein) to produce the antigen(s) or immunogenic fragment(s) thereof. The term polynucleotide encompasses both DNA and RNA sequences. Herein, the terms “nucleic acid”, “nucleic acid molecule” and “polynucleotide” are used interchangeably. Thus, the antigen derived from SARS-CoV-2 (e.g. SARS-CoV-2 spike protein) or an immunogenic fragment thereof may be encoded or expressed by a DNA or RNA vaccine.

The one or more polynucleotide expressing the one or more SARS-CoV-2 spike protein or immunogenic fragment thereof in a combined influenza-COVID-19 vaccine of the invention may express a spike protein from SARS-CoV-2 having at least 70%, at least 75%, at least 80%, at least 90%, at least 95%, at least 96%, at least 97%, at least 98%, at least 99% or more identity with SEQ ID NO: 1, or a fragment thereof, that has a common antigenic cross-reactivity with said spike protein. Preferably said one or more polynucleotide expresses one or more spike protein from SARS-CoV-2 having at least 90%, at least 95%, at least 96%, at least 97%, at least 98%, at least 99% or more identity with SEQ ID NO: 1, or a fragment thereof, that has a common antigenic cross-reactivity with said spike protein. More preferably, said one or more polynucleotide expresses one or more spike protein from SARS-CoV-2 having least 98%, at least 99% or more with SEQ ID NO: 1, or a fragment thereof, that has a common antigenic cross-reactivity with said spike protein. Said one or more polynucleotide may express a spike protein from SARS-CoV-2 comprising or consisting of SEQ ID NO: 1, or a fragment thereof, that has a common antigenic cross-reactivity with said spike protein. Multiple SARS-CoV-2 antigens (particularly one or more SARS-CoV-2 spike proteins) may be expressed by a polynucleotide or by multiple polynucleotides or a combination thereof. By way of non-limiting example, said one or more SARS-CoV-2 antigens (particularly one or more SARS-CoV-2 spike proteins) may be expressed by a single polynucleotide, or each of said SARS-CoV-2 antigens (particularly one or more SARS-CoV-2 spike proteins) may be expressed by separate polynucleotides.

Typically said polynucleotide comprises an isolated polynucleotide encoding a spike protein from SARS-CoV-2 having at least 90% identity with SEQ ID NO: 1, or a fragment thereof that has a common antigenic cross-reactivity with said spike protein, or any variant thereof as described herein. For example, the polynucleotide may encode an RBD of the SARS-CoV-2 spike protein, preferably wherein said RBD has at least 90% identity with SEQ ID NO: 15. Exemplary polynucleotides encoding the RBD are shown in SEQ ID NO: 13, and the codon-optimised sequence of SEQ ID NO: 14. Accordingly, a polynucleotide of the invention may comprise or consist of a nucleic acid sequence having at least 70%, at least 75%, at least 80%, at least 90%, at least 95%, at least 96%, at least 97%, at least 98%, at least 99% or more identity to SEQ ID NO: 13. Preferably a polynucleotide of the invention may comprise or consist of a nucleic acid sequence having at least 90%, at least 95%, at least 96%, at least 97%, at least 98%, at least 99% or more identity to SEQ ID NO: 13. More preferably, a polynucleotide of the invention may comprise or consist of a nucleic acid sequence having at least 98%, at least 99% or more identity to SEQ ID NO: 13. A polynucleotide of the invention may comprise or consist of the nucleic acid sequence of SEQ ID NO: 13.

The invention also encompasses polynucleotides encoding a variant spike protein from SARS-CoV-2, as described above, or fragments thereof that have common antigenic cross-reactivity with said variant spike protein. Said variant spike proteins typically have at least 90% identity with SEQ ID NO: 1, or a fragment thereof, such as the RBD of SEQ ID NO: 15.

The one or more polynucleotide (e.g. a DNA or RNA vaccine) encoding the one or more SARS-CoV-2 spike protein or immunogenic fragments thereof may be optimised for expression in a patient. The term “optimised” as used herein relates to optimisation for expression of the one or more SARS-CoV-2 spike protein or immunogenic fragment thereof, and includes both codon optimisation and/or other modifications to the polynucleotide (both in terms of the nucleic acid sequence and other modifications) which increase the level and/or duration of expression of the one or more SARS-CoV-2 spike protein from the polynucleotide within the patient, or which otherwise provide an advantage when expressing the one or more SARS-CoV-2 spike protein, or fragment thereof, from a DNA or RNA vaccine. The inventors have previously described optimised polynucleotides encoding SARS-CoV-2 spike proteins and fragments in UK Patent Application No. 2002166.3, which is herein incorporated by reference in its entirety.

Accordingly, one or more antigen derived from SARS-CoV-2 or an immunogenic fragment thereof, particularly one or more SARS-CoV-2 spike protein or immunogenic fragment thereof may be encoded by one or more polynucleotide (e.g. a DNA or RNA vaccine) comprising a nucleic acid sequence having at least 70%, at least 75%, at least 80%, at least 90%, at least 95%, at least 96%, at least 97%, at least 98%, at least 99% or more identity to any one of SEQ ID NOs: 2, 3, 4, 5, 6, 7, 8, 13, 14, 26, 27, 29, 30 or 32. Preferably said one or more polynucleotide comprises a nucleic acid sequence having at least 90%, at least 95%, at least 96%, at least 97%, at least 98%, at least 99% or more identity to any one of SEQ ID NOs: 2, 3, 4, 5, 6, 7, 8, 13, 14, 26, 27, 29, 30 or 32. More preferably, said one or more polynucleotide comprises a nucleic acid sequence having at least 98%, at least 99% or more identity to any one of SEQ ID NOs: 2, 3, 4, 5, 6, 7, 8, 13, 14, 26, 27, 29, 30 or 32. Said one or more polynucleotide may comprise the nucleic acid sequence of any one of SEQ ID NOs: 2, 3, 4, 5, 6, 7, 8, 13, 14, 26, 27, 29, 30 or 32. In addition, the 5′ cloning site, the 3′ cloning site, or the 5′ and 3′ cloning sites identified in any of SEQ ID NOs; 2, 3, 4, 5, 6, 7, 8, 13, 14, 26, 27, 29, 30 or 32, or any variant thereof as described herein, may be deleted in a polynucleotide (e.g. a DNA or RNA vaccine). Thus, the one or more polynucleotide (e.g. DNA or RNA vaccine) may comprise any one of SEQ ID NOs: 2, 3, 4, 5, 6, 7, 8, 13, 14, 26, 27, 29, 30 or 32, but lacking the 5′ cloning site, the 3′ cloning site, or the 5′ and 3′ cloning sites identified in any of SEQ ID NOs; 2, 3, 4, 5, 6, 7, 8, 13, 14, 26, 27, 29, 30 or 32. Alternatively, the 5′ cloning site, the 3′ cloning site, or the 5′ and 3′ cloning sites identified in any of SEQ ID NOs; 2, 3, 4, 5, 6, 7, 8, 13, 14, 26, 27, 29, 30, or 32, or any variant thereof as described herein, may be independently replaced with another appropriate cloning site. Suitable alternative cloning sites are well known in the art.

The invention particularly relates to antigens derived from SARS-CoV-2 or an immunogenic fragment that comprise or consist of an RBD of the SARS-CoV-2 spike protein. Accordingly, a polynucleotide of the invention may comprise or consist of a nucleic acid sequence having at least 70%, at least 75%, at least 80%, at least 90%, at least 95%, at least 96%, at least 97%, at least 98%, at least 99% or more identity to SEQ ID NO: 13, or to the codon-optimised sequence of SEQ ID NO: 14. Preferably a polynucleotide of the invention may comprise or consist of a nucleic acid sequence having at least 90%, at least 95%, at least 96%, at least 97%, at least 98%, at least 99% or more identity to SEQ ID NO: 13, or to the codon-optimised sequence of SEQ ID NO: 14. More preferably, a polynucleotide of the invention may comprise or consist of a nucleic acid sequence having at least 98%, at least 99% or more identity to SEQ ID NO: 13, or to the codon-optimised sequence of SEQ ID NO: 14. A polynucleotide of the invention may comprise or consist of the nucleic acid sequence of SEQ ID NO: 13, or the codon-optimised sequence of SEQ ID NO: 14.

The one or more polynucleotide (e.g. a DNA or RNA vaccine) according to the invention typically encodes at least one SARS-CoV-2 spike protein, or an immunogenic fragment thereof which: (a) retains the conformational epitopes present in the native SARS-CoV-2 spike protein; and/or (b) results in the production of neutralising antibodies specific for the spike protein or fragment thereof when said nucleic acid is administered to a patient.

The one or more polynucleotide (e.g. DNA or RNA vaccine) typically expresses at least one spike protein from SARS-CoV-2 or immunogenic fragment thereof, particularly at least one spike protein from SARS-CoV-2 or immunogenic fragment thereof as described herein (including in the form of a VLP or fusion protein).

The one or more polynucleotide (e.g. a DNA or RNA vaccine) according to the invention may be comprised in an expression construct to facilitate expression of the one or more SARS-CoV-2 spike protein or fragment thereof. Typically, in such an expression construct said one or more polynucleotide is operably linked to a suitable promoter(s). The one or more polynucleotide may be linked to a suitable terminator sequence(s). The one or more polynucleotide may be linked to both a promoter(s) and terminator(s). Suitable promoter and terminator sequences are well known in the art.

The one or more polynucleotide (e.g. DNA or RNA vaccine) may encode at least one SARS-CoV-2 spike protein or immunogenic fragment thereof which additionally comprises a leader sequence(s), for example to assist in the secretion of the at least one SARS-CoV-2 spike protein or immunogenic fragment thereof. Any suitable leader sequence may be used, including conventional leader sequences known in the art. Suitable leader sequences include human tissue plasminogen activator leader sequence (tPA), which is routinely used in viral and DNA based vaccines and for protein vaccines to aid secretion from mammalian cells.

The at least one SARS-CoV-2 spike protein or immunogenic fragment thereof may additionally comprise an N- or C-terminal tag, for example to assist in the recombinant production and/or purification of the at least one SARS-CoV-2 spike protein or immunogenic fragment thereof. Any N- or C-terminal tag may be used, including conventional tags known in the art. Suitable tags sequences include C-terminal hexa-histidine tags and the “C-tag” (the four amino acids EPEA at the C-terminus), which are commonly used in the art to aid purification from heterologous expression systems, e.g. insect cells, mammalian cells, bacteria, or yeast. In other embodiments, the at least one SARS-CoV-2 spike protein or immunogenic fragment thereof of the invention are purified from heterologous expression systems without the need to use a purification tag.

The at least one SARS-CoV-2 spike protein or immunogenic fragment thereof of the invention may comprise a leader sequence and/or a tag as defined herein.

### Viral Vectors, DNA Plasmids and RNA Vaccines

In a combined influenza-COVID-19 vaccine of the invention, the one or more antigen derived from SARS-CoV-2 (e.g. SARS-CoV-2 spike protein) or an immunogenic fragment thereof may be encoded or expressed by one or more viral vector, DNA vector (or DNA plasmid) or RNA vaccine. The term “vector” as used herein refers to a viral vector, a DNA vector (or DNA plasmid) or an RNA vaccine.

Said one or more viral vector, DNA vector (or DNA plasmid) or RNA vaccine may comprise one or more polynucleotide encoding at least one antigen derived from SARS-CoV-2 as described herein. Preferably, said one or more viral vector, DNA vector (or DNA plasmid) or RNA vaccine encodes at least one SARS-CoV-2 spike protein or immunogenic fragment thereof as described herein. Multiple SARS-CoV-2 antigens (particularly one or more SARS-CoV-2 spike proteins) may be expressed by a single viral vector, DNA vector (or DNA plasmid) or RNA vaccine or by multiple viral vectors, DNA vectors (or DNA plasmids) or RNA vaccines or a combination thereof. By way of non-limiting example, said one or more SARS-CoV-2 antigens (particularly one or more SARS-CoV-2 spike proteins) may be expressed by a single viral vector, DNA vector (or DNA plasmid) or RNA vaccine, or each of said SARS-CoV-2 antigens (particularly one or more SARS-CoV-2 spike proteins) may be expressed by a separate viral vector, DNA vector (or DNA plasmid) or RNA vaccine.

The one or more viral vector, a DNA vector (or DNA plasmid) or an RNA vaccine expressing the one or more SARS-CoV-2 spike protein or immunogenic fragment thereof in a combined influenza-COVID-19 vaccine of the invention may express at least one spike protein from SARS-CoV-2 having at least 70%, at least 75%, at least 80%, at least 90%, at least 95%, at least 96%, at least 97%, at least 98%, at least 99% or more identity with SEQ ID NO: 1, or a fragment thereof, that has a common antigenic cross-reactivity with said spike protein. Preferably said one or more viral vector, a DNA vector (or DNA plasmid) or an RNA vaccine expresses at least one spike protein from SARS-CoV-2 having at least 90%, at least 95%, at least 96%, at least 97%, at least 98%, at least 99% or more identity with SEQ ID NO: 1, or a fragment thereof, that has a common antigenic cross-reactivity with said spike protein. More preferably, said one or more viral vector, a DNA vector (or DNA plasmid) or an RNA vaccine expresses at least one spike protein from SARS-CoV-2 having least 98%, at least 99% or more with SEQ ID NO: 1, or a fragment thereof, that has a common antigenic cross-reactivity with said spike protein. Said one or more viral vector, a DNA vector (or DNA plasmid) or an RNA vaccine may express at least one spike protein from SARS-CoV-2 comprising or consisting of SEQ ID NO: 1, or a fragment thereof, that has a common antigenic cross-reactivity with said spike protein. In some preferred embodiments, the at least one spike protein from SARS-CoV-2 or immunogenic fragment thereof expressed by a vector of the invention is an RBD of the SARS-CoV-2 spike protein as defined herein, preferably wherein said RBD has at least 90% identity with SEQ ID NO: 15.

Typically said one or more viral vector, a DNA vector (or DNA plasmid) or an RNA vaccine expresses at least one spike protein from SARS-CoV-2 having at least 90% identity with SEQ ID NO: 1, or a fragment thereof that has a common antigenic cross-reactivity with said spike protein, or any variant thereof as described herein. A preferred fragment is an RBD with at least 90% identity to SEQ ID NO: 15.

The one or more viral vector, a DNA vector (or DNA plasmid) or an RNA vaccine expressing the at least one SARS-CoV-2 spike protein or immunogenic fragment thereof in a combined influenza-COVID-19 vaccine of the invention may express at least one spike protein or immunogenic fragment thereof as defined herein which further comprises a signal peptide(s). Typically said signal peptide directs secretion of the at least one SARS-CoV-2 spike protein or fragment thereof from a host cell of interest, such as cells in the patient to be treated.

The one or more viral vector, a DNA vector (or DNA plasmid) or an RNA vaccine expressing the at least one SARS-CoV-2 spike protein or immunogenic fragment thereof in a combined influenza-COVID-19 vaccine of the invention may further expresses one or more additional antigen or a fragment thereof. The spike protein or fragment thereof and the one or more additional antigen or fragment thereof may expressed as a fusion protein. Alternatively, separate vectors expressing the SARS-CoV-2 spike protein or fragment thereof and the one or more additional antigen or fragment thereof may be used. In such instances, said separate vectors may be used in combination, preferably simultaneously. The one or more additional antigen may be the same antigen or a different antigen from SARS-CoV-2, or a fragment thereof. More preferably, said one or more additional antigen is a different antigen from SARS-CoV-2, such as an antigen from the 2019-CoV capsid, membrane protein or envelope protein.

The one or more viral vector, a DNA vector (or DNA plasmid) or an RNA vaccine expressing the at least one SARS-CoV-2 spike protein or immunogenic fragment thereof in a combined influenza-COVID-19 vaccine of the invention may comprise any one or more polynucleotide or expression construct as defined herein, or any combination thereof.

The one or more vector(s) may be a viral vector. Such a viral vector may be an adenovirus (of a human serotype such as AdHu5, a simian serotype such as ChAd63, ChAdOX1 or ChAdOX2, or another form), an adeno-associated virus (AAV), or a poxvirus vector (such as a modified vaccinia Ankara (MVA)), or an adeno associated virus (AAV). ChAdOX1 and ChAdOX2 are disclosed in WO2012/172277 (herein incorporated by reference in its entirety). ChAdOX2 is a BAC-derived and E4 modified AdC68-based viral vector. Preferably said one or more viral vector is an AAV vector adenovirus. Other non-limiting examples of viral vectors include measles viral vectors, mumps viral vectors, rubella viral vectors, varicella viral vectors, polio viral vectors and yellow fever viral vectors.

Viral vectors are usually non-replicating or replication impaired vectors, which means that the viral vector cannot replicate to any significant extent in normal cells (e.g. normal human cells), as measured by conventional means—e.g. via measuring DNA synthesis and/or viral titre. Non-replicating or replication impaired vectors may have become so naturally (i.e. they have been isolated as such from nature) or artificially (e.g. by breeding in vitro or by genetic manipulation). There will generally be at least one cell-type in which the replication-impaired viral vector can be grown—for example, modified vaccinia Ankara (MVA) can be grown in CEF cells. By way of non-limiting example, the vector may be selected from a human or simian adenovirus or a poxvirus vector.

Typically, the one or more viral vector is incapable of causing a significant infection in an animal subject, typically in a mammalian subject such as a human or other primate.

The one or more vector(s) may be a DNA vector, such as a DNA plasmid. The one or more vector(s) may be an RNA vector, such as a mRNA vector or a self-amplifying RNA vector. The one or more DNA and/or RNA vector(s) of the invention is typically capable of expression in eukaryotic cells, particularly any host cell type described herein, or in a patient to be treated.

Typically the DNA and/or RNA vector(s) are capable of expression in a human, E. coli or yeast cell.

The one or more vector may be a phage vector, such as an AAV/phage hybrid vector as described in Hajitou et al., Cell 2006; 125(2) pp. 385-398; herein incorporated by reference.

The nucleic acid molecules and vectors of the invention may be made using any suitable process known in the art. Thus, the nucleic acid molecules may be made using chemical synthesis techniques. Alternatively, the nucleic acid molecules and vectors of the invention may be made using molecular biology techniques.

Vector(s) of the present invention may be designed in silico, and then synthesised by conventional polynucleotide synthesis techniques.

### Virus-Uke Particles

In a combined influenza-COVID-19 vaccine of the invention, the one or more antigen derived from SARS-CoV-2 (e.g. at least one SARS-CoV-2 spike protein) or an immunogenic fragment thereof may be comprised in a virus-like particle (VLP).

Virus-like particles (VLPs) are particles which resemble viruses but do not contain viral nucleic acid and are therefore non-infectious. They commonly contain one or more virus capsid or envelope proteins which are capable of self-assembly to form the VLP. VLPs have been produced from components of a wide variety of virus families (Noad and Roy (2003), Trends in Microbiology, 11:438-444; Grgacic et al., (2006), Methods, 40:60-65). Some VLPs have been approved as therapeutic vaccines, for example Engerix-B (for hepatitis B), Cervarix and Gardasil (for human papilloma viruses).

Multiple SARS-CoV-2 antigens (particularly one or more SARS-CoV-2 spike proteins) may be comprised in a VLP or a combination thereof. By way of non-limiting example, said one or more SARS-CoV-2 antigens (particularly one or more SARS-CoV-2 spike proteins) may be comprised in a single VLP, or each of said SARS-CoV-2 antigens (particularly one or more SARS-CoV-2 spike proteins) may be comprised in separate VLPs.

Accordingly, the one or more antigen derived from SARS-CoV-2 (e.g. at least one SARS-CoV-2 spike protein) or an immunogenic fragment thereof may be comprised in one or more VLP.

The one or more VLP comprising the at least one SARS-CoV-2 spike protein or immunogenic fragment thereof in a combined influenza-COVID-19 vaccine of the invention may comprise one or more spike protein from SARS-CoV-2 having at least 70%, at least 75%, at least 80%, at least 90%, at least 95%, at least 96%, at least 97%, at least 98%, at least 99% or more identity with SEQ ID NO: 1, or a fragment thereof, that has a common antigenic cross-reactivity with said spike protein. Preferably said one or more VLP comprises one or more spike protein from SARS-CoV-2 having at least 90%, at least 95%, at least 96%, at least 97%, at least 98%, at least 99% or more identity with SEQ ID NO: 1, or a fragment thereof, that has a common antigenic cross-reactivity with said spike protein. More preferably, said one or more VLP comprises one or more spike protein from SARS-CoV-2 having least 98%, at least 99% or more with SEQ ID NO: 1, or a fragment thereof, that has a common antigenic cross-reactivity with said spike protein. Said one or more VLP may comprise at least one spike protein from SARS-CoV-2 comprising or consisting of SEQ ID NO: 1, or a fragment thereof, that has a common antigenic cross-reactivity with said spike protein. In some preferred embodiments, the immunogenic fragment of the SARS-CoV-2 spike protein comprised in a VLP of the invention is an RBD of the SARS-CoV-2 spike protein as defined herein, preferably wherein said RBD has at least 90% identity with SEQ ID NO: 15.

Typically said one or more VLP comprises at least one spike protein from SARS-CoV-2 having at least 90% identity with SEQ ID NO: 1, or a fragment thereof that has a common antigenic cross-reactivity with said spike protein, or any variant thereof as described herein. A preferred fragment is an RBD with at least 90% identity to SEQ ID NO: 15.

The skilled person will understand that VLPs can be synthesized through the individual expression of viral structural proteins, which can then self-assemble into the virus-like structure. Combinations of structural capsid proteins from different viruses can be used to create recombinant VLPs. In additions, antigens or immunogenic fragments thereof can be fused to the surface of VLPs. By way of non-limiting example, antigens or immunogenic fragments thereof of the invention may be coupled to a VLP using the SpyCatcher-SpyTag system (as described by Brune, Biswas, Howarth).

Said one or more VLP may comprise one or more additional protein antigen. The one or more additional antigen may be the same antigen or a different antigen from SARS-CoV-2, or a fragment thereof. More preferably, said one or more additional antigen is a different antigen from SARS-CoV-2, such as an antigen from the SARS-CoV-2 capsid, membrane protein or envelope protein.

Said one or more VLP may comprise at least one fusion protein as described herein. Said one or more VLP may comprise a fusion protein of the SARS-CoV-2 spike protein or immunogenic fragment thereof with Hepatitis B surface antigen (HBSAg), human papillomavirus (HPV) 18 L1 protein, HPV 16 L1 protein and/or Hepatitis E P239, preferably Hepatitis B surface antigen.

Thus, said one or more VLP may be encoded by a polynucleotide which comprises or consists of a nucleic acid sequence having at least 70%, at least 75%, at least 80%, at least 90%, at least 95%, at least 96%, at least 97%, at least 98%, at least 99% or more identity to any one of SEQ ID NO: 3, 5, 6 or 8. Preferably said one or more VLP may be encoded by a polynucleotide which comprises or consists of a nucleic acid sequence having at least 90%, at least 95%, at least 96%, at least 97%, at least 98%, at least 99% or more identity to any one of SEQ ID NOs: 3, 5, 6 or 8. More preferably, said one or more VLP may be encoded by a polynucleotide which comprises or consists of a nucleic acid sequence having at least 98%, at least 99% or more identity to any one of SEQ ID NOs: 3, 5, 6 or 8. Said one or more VLP may be encoded by a polynucleotide which comprises or consists of a nucleic acid sequence of any one of SEQ ID NOs: 3, 5, 6 or 8.

A VLP of the invention may be encoded by a polynucleotide which comprises or consists of a nucleic acid sequence having at least 70%, at least 75%, at least 80%, at least 90%, at least 95%, at least 96%, at least 97%, at least 98%, at least 99% or more identity to any one of SEQ ID NOs: 26, 27, 29, 30 or 32. Preferably a VLP of the invention may be encoded by a polynucleotide which comprises or consists of a nucleic acid sequence having at least 90%, at least 95%, at least 96%, at least 97%, at least 98%, at least 99% or more identity to any one of SEQ ID NOs: 26, 27, 29, 30 or 32. More preferably, a VLP of the invention may be encoded by a polynucleotide which comprises or consists of a nucleic acid sequence having at least 98%, at least 99% or more identity to any one of SEQ ID NOs: 26, 27, 29, 30 or 32. A VLP of the invention may be encoded by a polynucleotide which comprises or consists of the nucleic acid sequence of any one of SEQ ID NOs: 26, 27, 29, 30 or 32.

Said one or more VLP may comprise or consist of an amino acid sequence having at least 70%, at least 75%, at least 80%, at least 90%, at least 95%, at least 96%, at least 97%, at least 98%, at least 99% or more identity to any one of SEQ ID NO: 9, 10, 11 or 12. Preferably said VLP may comprise or consist of an amino acid sequence having at least 90%, at least 95%, at least 96%, at least 97%, at least 98%, at least 99% or more identity to any one of SEQ ID NOs: 9, 10, 11 or 12. More preferably, said one or more VLP comprises or consists of an amino acid sequence having at least 98%, at least 99% or more identity to any one of SEQ ID NOs: 9, 10, 11 or 12. Said VLP may comprise or consist of an amino acid sequence of any one of SEQ ID NOs: 9, 10, 11 or 12.

A VLP of the invention may comprise or consist of an amino acid sequence having at least 70%, at least 75%, at least 80%, at least 90%, at least 95%, at least 96%, at least 97%, at least 98%, at least 99% or more identity to any one of SEQ ID NOs: 28, 31 or 33. Preferably a VLP of the invention may comprise or consist of an amino acid sequence having at least 90%, at least 95%, at least 96%, at least 97%, at least 98%, at least 99% or more identity to any one of SEQ ID NOs: 28, 31 or 33. More preferably, a VLP of the invention may comprises or consists of an amino acid sequence having at least 98%, at least 99% or more identity to any one of SEQ ID NOs: 28, 31 or 33. A VLP of the invention may comprise or consist of an amino acid sequence of any one of SEQ ID NOs: 28, 31 or 33.

The use of one or more VLP may increase the efficacy of the immunoprotective response induced by the SARS-CoV-2 spike protein or immunogenic fragment and/or may increase the duration of the immunoprotective response as defined herein.

### Fusion Proteins

In a combined influenza-COVID-19 vaccine of the invention, the one or more antigen derived from SARS-CoV-2 (e.g. one or more SARS-CoV-2 spike protein) or an immunogenic fragment thereof may be comprised in a fusion protein.

Accordingly, the one or more antigen derived from SARS-CoV-2 (e.g. one or more SARS-CoV-2 spike protein) or an immunogenic fragment thereof may be comprised in one or more fusion protein.

Multiple SARS-CoV-2 antigens (particularly one or more SARS-CoV-2 spike proteins) may be comprised in a fusion protein or a combination thereof. By way of non-limiting example, said one or more SARS-CoV-2 antigens (particularly one or more SARS-CoV-2 spike proteins) may be comprised in a single fusion protein, or each of said SARS-CoV-2 antigens (particularly one or more SARS-CoV-2 spike proteins) may be comprised in separate fusion proteins.

The one or more fusion protein comprising the at least one SARS-CoV-2 spike protein or immunogenic fragment thereof in a combined influenza-COVID-19 vaccine of the invention may comprise one or more spike protein from SARS-CoV-2 having at least 70%, at least 75%, at least 80%, at least 90%, at least 95%, at least 96%, at least 97%, at least 98%, at least 99% or more identity with SEQ ID NO: 1, or a fragment thereof, that has a common antigenic cross-reactivity with said spike protein. Preferably said one or more fusion protein comprises one or more spike protein from SARS-CoV-2 having at least 90%, at least 95%, at least 96%, at least 97%, at least 98%, at least 99% or more identity with SEQ ID NO: 1, or a fragment thereof, that has a common antigenic cross-reactivity with said spike protein. More preferably, said one or more fusion protein comprises one or more spike protein from SARS-CoV-2 having least 98%, at least 99% or more with SEQ ID NO: 1, or a fragment thereof, that has a common antigenic cross-reactivity with said spike protein. Said one or more fusion protein may comprise at least one spike protein from SARS-CoV-2 comprising or consisting of SEQ ID NO: 1, or a fragment thereof, that has a common antigenic cross-reactivity with said spike protein.

Typically said one or more fusion protein comprises at least one spike protein from SARS-CoV-2 having at least 90% identity with SEQ ID NO: 1, or a fragment thereof that has a common antigenic cross-reactivity with said spike protein, or any variant thereof as described herein.

In some preferred embodiments, the immunogenic fragment of the SARS-CoV-2 spike protein comprised in a fusion protein of the invention is an RBD of the SARS-CoV-2 spike protein as defined herein, preferably wherein said RBD has at least 90% identity with SEQ ID NO: 15.

A fusion protein of the invention typically also comprises a non-SARS-CoV-2 domain or element, typically a non-SARS-CoV-2 protein, polypeptide or peptide domain or element.

Said one or more fusion protein may comprise the at least one SARS-CoV-2 spike protein or immunogenic fragment thereof and one or more of: Hepatitis B surface antigen (HBSAg); human papillomavirus (HPV) 18 L1 protein; HPV 16 L1 protein; and/or Hepatitis E P239, preferably Hepatitis B surface antigen.

Said one or more fusion protein may be encoded by a polynucleotide which comprises or consists of a nucleic acid sequence having at least 70%, at least 75%, at least 80%, at least 90%, at least 95%, at least 96%, at least 97%, at least 98%, at least 99% or more identity to any one of SEQ ID NO: 3, 5, 6 or 8. Preferably said one or more fusion protein may be encoded by a polynucleotide which comprises or consists of a nucleic acid sequence having at least 90%, at least 95%, at least 96%, at least 97%, at least 98%, at least 99% or more identity to any one of SEQ ID NOs: 3, 5, 6 or 8. More preferably, said one or more fusion protein may be encoded by a polynucleotide which comprises or consists of a nucleic acid sequence having at least 98%, at least 99% or more identity to any one of SEQ ID NOs: 3, 5, 6 or 8. Said one or more fusion protein may be encoded by a polynucleotide which comprises or consists of a nucleic acid sequence of any one of SEQ ID NOs: 3, 5, 6 or 8.

A fusion protein of the invention may be encoded by a polynucleotide which comprises or consists of a nucleic acid sequence having at least 70%, at least 75%, at least 80%, at least 90%, at least 95%, at least 96%, at least 97%, at least 98%, at least 99% or more identity to any one of SEQ ID NOs: 26, 27, 29, 30 or 32. Preferably a fusion protein of the invention may be encoded by a polynucleotide which comprises or consists of a nucleic acid sequence having at least 90%, at least 95%, at least 96%, at least 97%, at least 98%, at least 99% or more identity to any one of SEQ ID NOs: 26, 27, 29, 30 or 32. More preferably, a fusion protein of the invention may be encoded by a polynucleotide which comprises or consists of a nucleic acid sequence having at least 98%, at least 99% or more identity to any one of SEQ ID NOs: 26, 27, 29, 30 or 32. A fusion protein of the invention may be encoded by a polynucleotide which comprises or consists of the nucleic acid sequence of any one of SEQ ID NOs: 26, 27, 29, 30 or 32.

Said one or more fusion protein may comprise or consist of an amino acid sequence having at least 70%, at least 75%, at least 80%, at least 90%, at least 95%, at least 96%, at least 97%, at least 98%, at least 99% or more identity to any one of SEQ ID NO: 9, 10, 11 or 12. Preferably said one or more fusion protein may comprise or consist of an amino acid sequence having at least 90%, at least 95%, at least 96%, at least 97%, at least 98%, at least 99% or more identity to any one of SEQ ID NOs: 9, 10, 11 or 12. More preferably, said one or more fusion protein may comprise or consist of an amino acid sequence having at least 98%, at least 99% or more identity to any one of SEQ ID NOs: 9, 10, 11 or 12. Said one or more fusion protein may comprise or consist of an amino acid sequence of any one of SEQ ID NOs: 9, 10, 11 or 12.

A fusion protein of the invention may comprise or consist of an amino acid sequence having at least 70%, at least 75%, at least 80%, at least 90%, at least 95%, at least 96%, at least 97%, at least 98%, at least 99% or more identity to any one of SEQ ID NOs: 28, 31 or 33. Preferably a fusion protein of the invention may comprise or consist of an amino acid sequence having at least 90%, at least 95%, at least 96%, at least 97%, at least 98%, at least 99% or more identity to any one of SEQ ID NOs: 28, 31 or 33. More preferably, a fusion protein of the invention may comprise or consist of an amino acid sequence having at least 98%, at least 99% or more identity to any one of SEQ ID NOs: 28, 31 or 33. A fusion protein of the invention may comprise or consist of an amino acid sequence of any one of SEQ ID NOs: 28, 31 or 33.

Said one or more fusion protein may preferably take the form of a VLP. Without being bound by theory, this is because HPSAg, HPV 18 L1 protein, HPB 16 L1 protein and Hepatitis E P239 protein are known to spontaneously form VLPs when expressed recombinantly, and this structure is retained when HPSAg, HPV 18 L1 protein, HPB 16 L1 protein and/or Hepatitis E P239 protein are present in fusion protein form combined with a SARS-CoV-2 spike protein of the invention (or immunogenic fragment thereof).

A fusion protein of the invention may comprise a linker (also referred to interchangeably herein as a linker peptide, a spacer or a spacer peptide). A linker may be used to join two or more functional domains of a fusion protein of the invention. Typically, where a linker is present, it is used to join the SARS-CoV-2 spike protein or immunogenic fragment thereof domain of the fusion protein to the non-SARS-CoV-2 spike protein domain of the fusion protein. Use of linkers in fusion proteins is routine in the art, and any conventional linker protein may be used in fusion proteins of the invention, provided that the resulting fusion protein retains the desired functional properties of the SARS-CoV-2 spike protein or immunogenic fragment thereof and the desired function properties of the non-2 SARS-CoV-2 spike protein domain.

A linker may be a short peptide of up to about 30 amino acids, such as about 5-30 amino acids, about 5-25 amino acids, about 5-20 amino acids, about 10-20 amino acids, about 5-15 amino acids or about 10-15 amino acids in length. In some embodiments, the linker is about 10, about 11, about 12, about 13, about 14, about 15, about 16, about 17, about 18, about 19 or about 20 amino acids in length.

In some embodiments a rigid linker may be used in fusion proteins of the invention. Rigid linkers are conventionally used when it is necessary to keep a fixed distance between the different domains/portions of a fusion protein and to maintain their independent functions. Rigid linkers may also be used when the spatial separation of the fusion protein domains is critical to preserve the stability or bioactivity of the fusion proteins. An empirical rigid linker with the sequence of A(EAAAK)ₙA (n=2-5) (SEQ ID NO: 16) displayed α-helical conformation, which is stabilized by Glu⁻-Lys⁺ salt bridges. A non-limiting example of a rigid linker is EAAAKEAAAKEAAAK (also referred to as (EAAAK)₃, SEQ ID NO: 18), which may be encoded by the nucleic acid sequence (SEQ ID NO: 17). Rigid linkers may be preferably used for expression of fusion proteins of the invention in mammalian cells, such as HEK 293 cells.

In some embodiments, flexible linkers may be used in fusion proteins of the invention. Flexible linkers are conventionally used when the joined domains require a certain degree of movement or interaction. Flexible linkers usually comprise or consist of small amino acid residues, such as glycine, threonine, arginine, serine, asparagine, glutamine, alanine, aspartic acid, proline, glutamic acid, lysine, leucine and/or valine, particularly glycine, serine, alanine, leucine and/or valine. Flexible linkers comprising or consisting of glycine, serine and/or alanine are preferred, with glycine and serine being particularly preferred. Accordingly, the most commonly used flexible linkers have sequences consisting primarily of stretches of Gly and Ser residues (“GS” linker), which comprise a sequence of (Gly-Gly-Gly-Gly-Ser)˜ (SEQ ID NO: 19). Non-limiting examples of GS linkers include GS5 or (GGGGS)₁ (SEQ ID NO: 20); GS10 or (GGGGS)₂ (SEQ ID NO: 21); GS15 or (GGGGS)₃ (SEQ ID NO: 23); GS20 or (GGGGS)₄ (SEQ ID NO: 24); and GS25 or (GGGGS)₅ (SEQ ID NO: 25). Preferably, GS15 may be used, which may be encoded by (SEQ ID NO: 22). Flexible linkers may be preferably used for expression of fusion proteins of the invention in bacterial cells, such as E. coli cells.

Any appropriate linker, such as the exemplary linkers described herein may be used with any fusion protein of the invention (comprising any SARS-CoV-2 spike protein or immunogenic fragment domain and any non-SARS-CoV-2 spike protein domain). By way of non-limiting example, a fusion protein of the invention may comprise or consist of HBSAg-(EAAAK)₃-RBD (SEQ ID NO: 28), or a variant with at least 90% sequence identity thereto, which may be encoded by (SEQ ID NO: 26 or 27), or a variant with at least 90% sequence identity thereto. By way of a further non-limiting example, a fusion protein of the invention may comprise or consist of HBSAg-(EAAAK)₃-full-length 2019-nCoV spike protein (SEQ ID NO: 33), or a variant with at least 90% sequence identity thereto, which may be encoded by SEQ ID NO: 32, or a variant with at least 90% sequence identity thereto. By way of further non-limiting example, a fusion protein of the invention may comprise or consist of HEV-GS15-RBD (SEQ ID NO: 31), or a variant with at least 90% sequence identity thereto, which may be encoded by (SEQ ID NO: 29 or 30), or a variant with at least 90% sequence identity thereto.

A fusion protein may preferably take the form of a VLP. Without being bound by theory, this is because HBSAg, HPV 18 L1 protein, HPB 16 L1 protein and Hepatitis E P239 protein are known to spontaneously form VLPs when expressed recombinantly, and this structure is retained when HBSAg, HPV 18 L1 protein, HPB 16 L1 protein and/or Hepatitis E P239 protein are present in fusion protein form combined with a SARS-CoV-2 spike protein of the invention (or immunogenic fragment thereof).

### Influenza Haemagglutinin (HA) and Neuraminidase (NA) Antigens

The combined influenza-COVID-19 vaccines of the invention comprise an influenza haemagglutinin (HA) or an immunogenic fragment thereof. Optionally, the combined influenza-COVID-19 vaccines of the invention may further comprise an influenza neuraminidase (NA) or an immunogenic fragment thereof.

An immunogenic fragment of HA has a common antigenic cross-reactivity with the HA from which it is derived. Similarly, an immunogenic fragment of NA has a common antigenic cross-reactivity with the NA from which it is derived.

The influenza HA or immunogenic fragment thereof (and optionally the influenza NA or immunogenic fragment thereof) may present in a combined influenza-COVID-19 vaccine in any appropriate form.

The influenza HA or immunogenic fragment thereof and/or the influenza NA or immunogenic fragment thereof will typically be prepared from influenza virions but, as an alternative, these antigens may be provided in other forms, such as polynucleotides, viral vector, a DNA vector (or DNA plasmid) or an RNA vaccine, VLPs and fusion proteins.

The general disclosure herein in relation to polynucleotides, viral vector, a DNA vector (or DNA plasmid) or an RNA vaccine, VLPs and fusion proteins is also applicable to the influenza HA or immunogenic fragment thereof and the influenza NA or immunogenic fragment thereof as described herein. Any general disclosure herein in relation to polynucleotides, viral vector, a DNA vector (or DNA plasmid) or an RNA vaccine, VLPs and fusion proteins in the context of antigens derived from SARS-Cov-2 (e.g. SARS-CoV-2 spike protein) applies equally and without restriction to the influenza HA or immunogenic fragment thereof and the influenza NA or immunogenic fragment thereof as described herein.

As described herein, (a) the influenza HA or immunogenic fragment thereof may be (i) comprised in an inactivated influenza virion; (ii) a recombinant HA or immunogenic fragment thereof; (iii) a fusion protein comprising HA or an immunogenic fragment thereof; or (iv) encoded by an RNA or DNA vaccine.

As described herein, (a) the influenza NA or immunogenic fragment thereof may be (i) comprised in an inactivated influenza virion; (ii) a recombinant NA or immunogenic fragment thereof; (iii) a fusion protein comprising NA or an immunogenic fragment thereof; or (iv) encoded by an RNA or DNA vaccine.

The influenza HA or immunogenic fragment thereof and/or the influenza NA or immunogenic fragment thereof may take the form of an existing influenza vaccine. The influenza HA or immunogenic fragment thereof and/or the influenza NA or immunogenic fragment thereof may take the form of a live (attenuated or vectored) vaccine, an inactivated vaccine or a subunit vaccine. Inactivated influenza vaccines include both inactivated whole virion vaccines and inactivated split virion vaccines, whole virion inactivated vaccines are preferred. Split virions are obtained by treating virions with detergents (e.g. ethyl ether, polysorbate 80, deoxycholate, tri-N-butyl phosphate, Triton X-100, Triton N101, cetyltrimethylammonium bromide, Tergitol NP9, etc.) to produce subvirion preparations. Methods of splitting influenza viruses are well known in the art.

An inactivated vaccine may be generated by any appropriate means. Conventional means for inactivating influenza virions include treatment with an effective amount of one or more of the following agents: detergents, formaldehyde, formalin, β-propiolactone, or UV light. Additional chemical means for inactivation include treatment with methylene blue, psoralen, carboxyfullerene (C60) or a combination of any thereof. Other methods of viral inactivation are known in the art, such as for example binary ethylamine, acetyl ethyleneimine, or gamma irradiation.

The combined influenza-COVID-19 vaccines of the invention may comprise or be produced using any influenza vaccine, including any commercially available influenza vaccine, a universal influenza vaccine and/or a pandemic influenza vaccine.

Typically influenza virus strains for use in vaccines change from season to season. In the current inter-influenza pandemic period, vaccines typically include two influenza A strains (H1N1 and H3N2) and one influenza B strain (B/Colorado/06/2017-like (Victoria lineage) virus), and trivalent vaccines against seasonal influenza (seasonal trivalent influenza vaccines) are typical. Quadrivalent vaccines against seasonal influenza (seasonal quadrivalent influenza vaccines) are also in common usage. Currently the seasonal quadrivalent influenza vaccines include the same strains as the seasonal trivalent influenza vaccines, with the inclusion of an additional influenza B strain (B/Phuket/3073/2013-like virus (Yamagata lineage)). Any seasonal influenza vaccine, including seasonal trivalent and quadrivalent influenza vaccines may be comprised in or used to produce the combined influenza-COVID-19 vaccines of the invention. Regulatory approved seasonal influenza vaccines are identified on the websites Centers for Disease Control and Prevention (CDC) (the CDC 2019-2020 list is provided here: https://www.cdc.gov/flu/professionals/acip/summary/summary-recommendations.htm#composition) and the European Medicines Agency (EMA).

Alternatively, a pandemic influenza vaccine may be comprised in or used to produce the combined influenza-COVID-19 vaccines of the invention. Pandemic influenza vaccines are raised against pandemic influenza strains, which are strains to which the vaccine recipient and the general human population are immunologically naïve, such as H2, H5, H7 or H9 subtype strains (in particular of influenza A virus). Pandemic influenza virus strains often arise in non-human species which then jump the species barrier to humans. A recent example of a potential pandemic influenza strain is the genotype 4 (G4) Eurasian avian-like (EA) H1N1 swine influenza strain. The combined influenza-COVID-19 vaccines of the invention may comprise an influenza component which is directed to such species-jumping pandemic strains, such as G4 EA H1N1. Pandemic influenza vaccines may be monovalent or may be based on a trivalent vaccine, supplemented by a pandemic strain. Monovalent pandemic influenza vaccines may be preferred.

A universal influenza vaccine may be comprised in or used to produce the combined influenza-COVID-19 vaccines of the invention. Examples of universal influenza vaccines under development include subunit vaccines and two-stage vaccines comprising a priming DNA vaccine and a live vectored vaccine.

Depending on the season and on the nature of the HA and/or NA included in the vaccine, the influenza component of the combined influenza-COVID-19 vaccines of the invention may protect against one or more of influenza A virus hemagglutinin subtypes H1, H2, H3, H4, H5, H6, H7, H8, H9, H10, H11, H12, H13, H14, H15 or H16. The invention may protect against one or more of influenza A virus NA subtypes N1, N2, N3, N4, N5, N6, N7, N8 or N9.

The influenza component of the combined influenza-COVID-19 vaccines of the invention may include HA and/or NA (or immunogenic fragments thereof) from one or more (e.g. 1, 2, 3, 4 or more) influenza strains, including influenza A virus and/or influenza B virus.

The viruses used as the source of the influenza HA and/or NA or the influenza vaccines which form the influenza component of the combined influenza-COVID-19 vaccines can be grown either on eggs or on cell culture. The current standard method for influenza virus growth uses specific pathogen-free (SPF) embryonated hen eggs, with virus being purified from the egg contents (allantoic fluid). More recently, however, viruses have been grown in animal cell culture and, for reasons of speed and patient allergies, this growth method is preferred. If egg-based viral growth is used then one or more amino acids may be introduced into the allantoid fluid of the egg together with the virus. When cell culture is used, the viral growth substrate will typically be a cell line of mammalian origin. Suitable mammalian cells of origin include, but are not limited to, hamster, cattle, primate (including humans and monkeys) and dog cells. Various cell types may be used, such as kidney cells, fibroblasts, retinal cells, lung cells, etc. Suitable cell lines include, but are not limited to: MDCK; CHO; 293T; BHK; Vero; MRC-5; PER.C6; WI-38; etc. Preferred mammalian cell lines for growing influenza viruses include: MDCK cells derived from Madin Darby canine kidney which are available e.g. from the American Type Cell Culture (ATCC) collection as CCL-34. Derivatives of the MDCK cell line may also be used.

Where virus has been grown on a mammalian cell line then the composition will advantageously be free from egg proteins (e.g. ovalbumin and ovomucoid) and from chicken DNA, thereby reducing allergenicity.

### Compositions and Therapeutic Indications

As described herein, the present inventors have demonstrated that vaccine compositions comprising SARS-CoV-2 antigens, particularly SARS-CoV-2 spike protein can be successfully combined with influenza virus vaccines, to generate robust antibody responses to both SARS-CoV-2 and influenza. Thus, the present inventions have surprisingly demonstrated that it is possible to produce combined influenza-COVID-19 vaccines with none of the expected problems of vaccine component suppression which are common in the production of combination vaccine products.

Accordingly, the present invention provides a combined influenza-COVID-19 vaccine as described herein. The invention provides a composition comprising (i) an influenza HA antigen or immunogenic fragment thereof; (ii) one or more antigen derived from SARS-CoV-2 (particularly at least one SARS-CoV-2 spike protein) or an immunogenic fragment thereof; and optionally (iii) an influenza NA antigen or immunogenic fragment thereof; wherein said composition is capable of inducing an immune response against SARS-CoV-2 (particularly against SARS-CoV-2 spike protein) and influenza (particularly influenza HA and optionally NA). The invention also provides the use of such a composition as a vaccine.

The invention also provides a vaccine composition comprising (i) an influenza HA antigen or immunogenic fragment thereof; (iii) one or more antigen derived from SARS-CoV-2 (particularly at least one SARS-CoV-2 spike protein) or an immunogenic fragment thereof; and optionally (iii) an influenza NA antigen or immunogenic fragment thereof. The vaccine composition may optionally comprise a pharmaceutically acceptable excipient, diluent, carrier, propellant, salt and/or additive.

The vaccine composition may comprise at least two different antigens derived from SARS-CoV-2 or immunogenic fragments thereof according to the invention, and/or at least two different polynucleotide molecules encoding at least two different antigens derived from SARS-CoV-2 or immunogenic fragments, as described herein. By way of non-limiting example, the vaccine composition may comprise a polynucleotide encoding a SARS-CoV-2 spike protein and a polynucleotide encoding a SARS-CoV-2 membrane protein.

The vaccine composition may comprise at least two different antigens derived from influenza or immunogenic fragments thereof according to the invention, and/or at least two different polynucleotide molecules encoding at least two different antigens derived from influenza or immunogenic fragments, as described herein. Typically the vaccine composition comprises an influenza HA antigen or immunogenic fragment thereof and optionally an influenza NA antigen or immunogenic fragment thereof. As the influenza component of the combined influenza-COVID-19 vaccines of the invention is typically provided by a live (attenuated or vectored) or inactivated influenza vaccine comprising whole or split influenza virions, other influenza antigens may also be included.

The present invention also provides a method of stimulating or inducing an immune response in a patient using a combined influenza-COVID-19 vaccine or composition of the invention (as described above). The vaccines and compositions of the present invention typically stimulate or induce an immune response and/or protection against both influenza and COVID-19.

Said method of stimulating or inducing an immune response in a subject may comprise administering a combined influenza-COVID-19 vaccine or composition of the invention (as described above) to a subject.

In the context of the therapeutic uses and methods, a “subject” is any animal subject that would benefit from stimulation or induction of an immunoprotective response against SARS-CoV-2 and influenza. Typical animal subjects are mammals, such as primates, for example, humans.

Thus, the present invention provides a method for treating or preventing SARS-CoV-2 infection (COVID-19) and influenza infection. Said method typically comprises the administration of a combined influenza-COVID-19 vaccine or composition of the invention to a subject in need thereof.

The present invention also provides a combined influenza-COVID-19 vaccine or composition of the invention for use in prevention or treatment of SARS-CoV-2 infection.

The present invention also provides the use of (i) one or more polynucleotide, expression construct, viral vector, DNA plasmid or RNA vaccine which expresses one or more SARS-CoV-2 spike protein or immunogenic fragment thereof, or one or more SARS-CoV-2 spike protein or immunogenic fragment thereof, one or more SARS-CoV-2 vaccine composition of the invention; and (ii) an influenza HA or immunogenic fragment thereof (and optionally an influenza NA or immunogenic fragment thereof), preferably comprised in an influenza vaccine as described herein, for the manufacture of a medicament for the prevention or treatment of SARS-CoV-2 infection and influenza infection.

As used herein, the term “treatment” or “treating” embraces therapeutic or preventative/prophylactic measures, and includes post-infection therapy and amelioration of a SARS-CoV-2 infection and influenza infection. The terms “therapy” and “therapeutic” embrace prophylactic therapy.

As used herein, the term “preventing” includes preventing the initiation of infection by SARS-CoV-2 and influenza and/or reducing the severity or intensity of an infection by SARS-CoV-2 and influenza. The term “preventing” includes inducing or providing protective immunity against infection by SARS-CoV-2 and influenza infection. Immunity to infection by a SARS-CoV-2 and influenza infection may be quantified using any appropriate technique, examples of which are known in the art.

Preferred compositions of the invention satisfy 1, 2 or 3 of the CPMP criteria for efficacy. In adults (18-60 years), these criteria are: (1) ≥70% seroprotection; (2) ≥40% seroconversion; and/or (3) a GMT increase of ≥2.5-fold. In elderly (>60 years), these criteria are: (1) ≥60% seroprotection; (2) ≥30% seroconversion; and/or (3) a GMT increase of ≥2-fold.

These criteria are based on open label studies with at least 50 patients.

A combined influenza-COVID-19 vaccine or composition of the invention as defined herein may be administered to a subject (typically a mammalian subject such as a human or other primate) already having a SARS-CoV-2 infection and/or an influenza infection, a condition or symptoms associated with infection by SARS-CoV-2 and/or influenza infection, to treat or prevent infection by SARS-CoV-2 and or influenza. For example, the subject may be suspected of having come in contact with SARS-CoV-2 or influenza, or has had known contact with SARS-CoV-2 or influenza, but is not yet showing symptoms of exposure.

When administered to a subject (e.g. a mammal such as a human or other primate) that already has a SARS-CoV-2 infection and/or influenza infection, or is showing symptoms associated with a SARS-CoV-2 infection and/or influenza infection, the combined influenza-COVID-19 vaccine or composition of the invention as defined herein can cure, delay, reduce the severity of, or ameliorate one or more symptoms, and/or prolong the survival of a subject beyond that expected in the absence of such treatment.

Alternatively, a combined influenza-COVID-19 vaccine or composition of the invention as defined herein may be administered to a subject (e.g. a mammal such as a human or other primate) who ultimately may be infected with SARS-CoV-2 and/or influenza, in order to prevent, cure, delay, reduce the severity of, or ameliorate one or more symptoms of said SARS-CoV-2 infection and/or influenza, or in order to prolong the survival of a subject beyond that expected in the absence of such treatment, or to help prevent that subject from transmitting a SARS-CoV-2 infection and/or influenza infection.

The treatments and preventative therapies of the present invention are applicable to a variety of different subjects of different ages. In the context of humans, the therapies are applicable to children (e.g. infants, children under 5 years old, older children or teenagers) and adults. In the context of other animal subjects (e.g. mammals such as primates), the therapies are applicable to immature subjects and mature/adult subjects. As used herein, the term “preventing” includes preventing the initiation of SARS-CoV-2 infection and/or influenza infection; and/or reducing the severity or intensity of a SARS-CoV-2 infection and/or influenza infection. The term “preventing” includes inducing or providing protective immunity against SARS-CoV-2 infection and/or influenza infection. Immunity to SARS-CoV-2 infection and/or influenza infection may be quantified using any appropriate technique, examples of which are known in the art.

As used, herein, a “vaccine” is a formulation that, when administered to an animal subject such as a mammal (e.g. a human or other primate) stimulates a protective immune response against SARS-CoV-2 infection and/or influenza infection. The immune response may be a humoral and/or cell-mediated immune response. A vaccine of the invention can be used, for example, to protect a subject from the effects of SARS-CoV-2 infection and/or influenza infection.

As described herein, the evidence available to-date indicates that immunity following SARS-CoV-2 infection may be relatively short-lived. Therefore, the invention provides the means of boosting immunity to SARS-CoV-2 infection by regular repeated administration of COVID-19/SARS-CoV-2 vaccine, in particular a combined influenza-COVID-19 vaccine of the invention. This repeated administration may use or be integrated into existing public health programs/schedules for seasonal influenza vaccination.

Accordingly, the invention provides a combined influenza-COVID-19 vaccine of the invention for use in the treatment and/or prevention of COVID-19 and influenza, wherein the combined vaccine is for administration at intervals of about six months, about seven months, about eight months, about nine months, about ten months, about 11 months, about 12 months, about 13 months, about 14 months or about 15 months. Preferably the combined vaccine is for administration at intervals of about 11 months, about 12 months, about 13 months, most preferable about 12 months. The invention also provides a method of immunising a subject against both influenza and COVID-19 comprising administering to said subject a therapeutically effective amount of a combined influenza-COVID-19 vaccine of the invention at these same intervals. The invention also provides the use of an influenza HA or an immunogenic fragment thereof; an antigen derived from SARS-CoV-2 or an immunogenic fragment thereof, and optionally an influenza NA or an immunogenic fragment thereof in the manufacture of a medicament for use in the treatment and/or prevention of COVID-19 and influenza, wherein said medicament is for administration at these same intervals.

The combined vaccine may be administered at an interval as described herein at least twice, at least five times, at least ten times, at least 15 times, at least 20 times or more.

The combined vaccine may be administered at an interval as described herein for a duration of at least two years, at least five years, at least ten years or more, up to the lifetime of a patient.

### Pharmaceutical Compositions and Formulations

The term “vaccine” is herein used interchangeably with the terms “therapeutic/prophylactic composition”, “formulation” or “medicament”.

The vaccine of the invention (as defined above) can be combined or administered in addition to a pharmaceutically acceptable carrier. Alternatively or in addition the vaccine of the invention can further be combined with one or more of a salt, excipient, diluent, adjuvant, immunoregulatory agent and/or antimicrobial compound.

Pharmaceutically acceptable salts include acid addition salts formed with inorganic acids such as, for example, hydrochloric or phosphoric acids, or with organic acids such as acetic, oxalic, tartaric, maleic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Administration of immunogenic compositions, therapeutic formulations, medicaments and prophylactic formulations (e.g. vaccines) is generally by conventional routes e.g. intravenous, subcutaneous, intraperitoneal, or mucosal (particularly nasal) routes. The administration may be by parenteral injection, for example, a subcutaneous, intradermal or intramuscular injection.

Accordingly, immunogenic compositions, therapeutic formulations, medicaments and prophylactic formulations (e.g. vaccines) of the invention are typically prepared as injectables, either as liquid solutions or suspensions. Solid forms suitable for solution in, or suspension in, liquid prior to injection may alternatively be prepared. The preparation may also be emulsified, or the peptide encapsulated in liposomes or microcapsules.

The active immunogenic ingredients (such as the SARS-CoV-2 spike proteins, fragments thereof, nucleic acids encoding said spike proteins, expression vectors, virial vectors, DNA plasmids, RNA vaccines, fusion proteins and vaccine compositions and the influenza HA and/or NA antigens or influenza vaccines as described herein) are often mixed with carriers, diluents, excipients or similar which are pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH buffering agents, and/or adjuvants which enhance the effectiveness of the vaccine.

Generally, the carrier, diluent, excipient or similar is a pharmaceutically-acceptable carrier. Non-limiting examples of pharmaceutically acceptable carriers include water, saline, and phosphate-buffered saline. In some embodiments, however, the composition is in lyophilized form, in which case it may include a stabilizer, such as BSA. In some embodiments, it may be desirable to formulate the composition with a preservative, such as thiomersal or sodium azide, to facilitate long term storage.

Examples of buffering agents include, but are not limited to, sodium succinate (pH 6.5), and phosphate buffered saline (PBS; pH 6.5 and 7.5).

Additional formulations which are suitable for other modes of administration include suppositories and, in some cases, oral formulations or formulations suitable for distribution as aerosols. For suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders.

### Adjuvants

Whilst conventional influenza vaccines do not comprise an adjuvant, the combined influenza-COVID-19 vaccine of the invention may further comprise an adjuvant. Said adjuvant may be a stimulator of cellular (Th1) and/or humoral (Th2) immune responses.

Examples of additional adjuvants which may be effective include but are not limited to: complete Freunds adjuvant (CFA), Incomplete Freunds adjuvant (IFA), Saponin, a purified extract fraction of Saponin such as Quil A, a derivative of Saponin such as QS-21, lipid particles based on Saponin such as ISCOM/ISCOMATRIX, E. coli heat labile toxin (LT) mutants such as LTK63 and/or LTK72, aluminium hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryl oxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion, the MF59 formulation developed by Novartis, and the AS02, AS01, AS03 and AS04 adjuvant formulations developed by GSK Biologicals (Rixensart, Belgium). Adjuvants typically present in a combined influenza-COVID-19 vaccine of the invention may be selected from squalene oil-in-water emulsions, aluminium salts and monophosphoryl Lipid A (MPL). Particularly preferred adjuvants include Addavax®, 5% squalene (MF59), MPL and aluminium hydroxide and aluminium phosphate gel.

### Kits

The invention provides kits comprising the combined influenza-COVID-19 vaccines of the invention, optionally with instructions for use. Any adjuvant may be contained separate from the combined vaccine within the kit or may be combined with the combined vaccine. The combined vaccine in a kit may be ready for use (e.g. including the adjuvant), or may be ready for extemporaneous preparation (e.g. to incorporate the adjuvant) at the time of delivery. This extemporaneous arrangement allows the adjuvant and the antigen to be kept separately until the time of use, which is particularly useful when using an oil-in-water emulsion adjuvant.

The invention also provides kits of parts comprising the SARS-CoV-2 component of the combined vaccine and the influenza component of the combined vaccine. The two components may be separate within the kit. Any adjuvant may be contained separate within the kit or may be combined with either the SARS-CoV-2 component or the influenza component. In such instances, the components may be mixed prior to administration to a patient, or the components may remain separate but be administered to a patient substantially at the same time or simultaneously.

The invention also provides kits of parts comprising the SARS-CoV-2 component of the combined vaccine and an adjuvant, preferably a squalene oil-in-water emulsion, an aluminium salt or MPL, more preferably Addavax®, MF59, MPL or aluminium hydroxide and aluminium phosphate gel. Optionally the kit of parts may include instructions regarding the combining of the SARS-CoV-2 component and adjuvant with an existing influenza vaccine (examples of which are described herein) and administering the combined influenza-COVID-19 vaccine as a single unit, or administering the mixed SARS-CoV-2 and adjuvant to a patient substantially at the same time or simultaneously to the influenza vaccine.

The SARS-CoV-2 component and/or the influenza component in a kit may be ready for use, or may be ready for extemporaneous preparation at the time of delivery. This extemporaneous arrangement allows the adjuvant and the SARS-CoV-2 and/or influenza components to be kept separately until the time of use, which is particularly useful when using an oil-in-water emulsion adjuvant.

Where a vaccine is prepared extemporaneously, its components are physically separate from each other within the kit, and this separation can be achieved in various ways. For instance, the two components may be in two separate containers, such as vials. The contents of the two vials can then be mixed e.g. by removing the contents of one vial and adding them to the other vial, or by separately removing the contents of both vials and mixing them in a third container. By way of non-limiting example, one of the kit components is in a syringe and the other is in a container such as a vial. The syringe can be used (e.g. with a needle) to insert its contents into the second container for mixing, and the mixture can then be withdrawn into the syringe. The mixed contents of the syringe can then be administered to a patient, typically through a new sterile needle. Packing one component in a syringe eliminates the need for using a separate syringe for patient administration. By way of further non-limiting example, the two components of a vaccine are held together but separately in the same syringe e.g. a dual-chamber syringe. When the syringe is actuated (e.g. during administration to a patient) then the contents of the two chambers are mixed. This arrangement avoids the need for a separate mixing step at the time of use.

Where a vaccine is prepared extemporaneously (either by mixing the combined vaccine with an adjuvant, or by mixing the SARS-CoV-2 component and the influenza component, optionally with an adjuvant), its components will generally be in aqueous form. In some arrangements, a component (typically the combined vaccine or the SARS-CoV-2 component and/or the influenza component of said vaccine, rather than the adjuvant component) is in dry form (e.g. in a lyophilised form), with one or more of the other components being in aqueous form. The components can be mixed in order to reactivate the dry component and give an aqueous composition for administration to a patient.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics: 1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

Alignment score for determining sequence identity

BLOSUM62 table

A R N D C Q E G H I L K M F P S T W Y V

### A4

### R −1 5

### N −2 0 6

### D −2 −2 1 6

### C0 −3 −3 −3 9

### Q −1 1 0 0 −3 5

### E −1 0 0 2 −4 2 5

### G 0 −2 0 −1 −3 −2 −2 6

### H −2 0 1 −1 −3 0 0 −2 8

### I −1 −3 −3 −3 −1 −3 −3 −4 −3 4

### L −1 −2 −3 −4 −1 −2 −3 −4 −3 2 4

### K −1 2 0 −1 −3 1 1 −2 −1 −3 −2 5

### M −1 −1 −2 −3 −1 0 −2 −3 −2 1 2 −1 5

### F −2 −3 −3 −3 −2 −3 −3 −3 −1 0 0 −3 0 6

### P −1 −2 −2 −1 −3 −1 −1 −2 −2 −3 −3 −1 −2 −4 7

### S 1 −1 1 0 −1 0 0 0 −1 −2 −2 0 −1 −2 −1 4

### T 0 −1 0 −1 −1 −1 −1 −2 −2 −1 −1 −1 −1 −2 −1 1 5

### W −3 −3 −4 −4 −2 −2 −3 −2 −2 −3 −2 −3 −1 1 −4 −3 −2 11

### Y −2 −2 −2 −3 −2 −1 −2 −3 2 −1 −1 −2 −1 3 −3 −2 −2 2 7

### V 0 −3 −3 −3 −1 −2 −2 −3 −3 3 1 −2 1 −1 −2 −2 0 −3 −1 4

The percent identity is then calculated as:

$$\frac{Total number of identical matches}{[ \begin{matrix}length of the longer sequence plus the number of gaps introduced \\ into the longer sequence in order to align the two sequences\end{matrix} ]} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

### Conservative Amino Acid Substitutions

Basic: arginine
lysine
histidine
   Acidic: glutamic acid
aspartic acid
   Polar: glutamine
asparagine
   Hydrophobic: leucine
isoleucine
valine
   Aromatic: phenylalanine
tryptophan
tyrosine
   Small: glycine
alanine
serine
threonine
methionine

In addition to the 20 standard amino acids, non-standard amino acids (such as 4-hydroxyproline, 6-N-methyl lysine, 2-aminoisobutyric acid, isovaline and a-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues in the SARS-CoV-2 antigens of the invention. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allothreonine, methyl-threonine, hydroxy-ethylcysteine, hydroxyethylhomo-cysteine, nitroglutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90: 10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labelling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30: 10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

The following Examples illustrate the invention.

### EXAMPLES

### Example 1: Comparison of Immunogenicity of a Trivalent Commercial Flu Vaccine (Addavax Adjuvanted) Alone, and a COVID-19 Vaccine (RBD-HBs Conjugated Produced in HEK Cells and Addavax Adjuvanted) Alone with a Combined Flu-Covid-19 Vaccine (Addavax Adjuvanted)

Three vaccine preparations were prepared:
1. Commercial Flu vaccine 3 μg/ml (split type) Addavax adjuvanted (20 μl/ml)
2. Covid-19 vaccine (RBD-HBs conjugated, produced in HEK cells) 3 μg/ml Addavax adjuvanted (20 μl/ml)
3. Combined Flu-Covid-19 vaccine (3 μg each component/ml) Addavax adjuvanted (20 μl/

Three groups of 5 Balb/c mice were vaccinated with 0.5 ml of each the above vaccines (day 0). Serum samples were taken from the mice on day 0 and 14.

Antibody titres were measured by ELISA against the receptor binding domain (RBD) of the SARS-CoV-2 spike protein (COVID-19 antigen) and against H1N1, H3N2 and B antigens of influenza virus. Antibody titres against influenza antigens are shown in Table 1. Antibody titres against the SARS-CoV-2 spike protein are shown in Table 2. All vaccines elicited a strong antibody response. The use of an adjuvant containing combined influenza-COVID-19 vaccine was able to elicit strong antibody responses against both influenza and the SARS-CoV-2 spike protein, with no evidence of component suppression.

TABLE 1
Antibody titres against influenza antigens

| Vaccine Group | ELISA Antibody Titre against |
|---|---|
| (5 Balb/c mice per group) | Influenza Antigens |
| PBS control | 0 |
| COVID-19 day 0 | 0 |
| COVID-19 day 14 | 0 |
| Flu H1N1 day 0 | 0 |
| Flu H1N1 day 14 | 67.1 |
| Flu H3N2 day 0 | 0 |
| Flu H3N2 day 14 | 43.1 |
| Flu B day 0 | 0 |
| Flu B day 14 | 40.5 |
| COVID-19 + Flu H1N1 day 0 | 0 |
| COVID-19 + Flu H1N1 day 14 | 69.3 |
| COVID-19 + Flu H3N2 day 0 | 0 |
| COVID-19 + Flu H3N2 day 14 | 50.3 |
| COVID-19 + Flu B day 0 | 0 |
| COVID-19 + Flu B day 14 | 39.4 |

TABLE 2
Antibody titres against SARS-COV-2 spike protein

| Vaccine Group | ELISA Antibody Titre against |
|---|---|
| (5 Balb/c mice per group) | SARS-Cov-2 spike protein |
| PBS control | 0 |
| COVID-19 day 0 | 0 |
| COVID-19 day 14 | 3.2 |
| Flu H1N1 day 0 | 0 |
| Flu H1N1 day 14 | 0 |
| Flu H3N2 day 0 | 0 |
| Flu H3N2 day 14 | 0 |
| Flu B day 0 | 0 |
| Flu B day 14 | 0 |
| COVID-19 + Flu H1N1 day 0 | 0 |
| COVID-19 + Flu H1N1 day 14 | 3.5 |
| COVID-19 + Flu H3N2 day 0 | 0 |
| COVID-19 + Flu H3N2 day 14 | 3.6 |
| COVID-19 + Flu B day 0 | 0 |
| COVID-19 + Flu B day 14 | 3.4 |

### Example 2: Comparison of Immunogenicity of a Commercial Flu Vaccine (Vaxigrip) Alone, and a COVID-19 Vaccine (Full-Size Spike Protein Conjugated to HBSAg) Alone with a Combined Flu-Covid-19 Vaccine

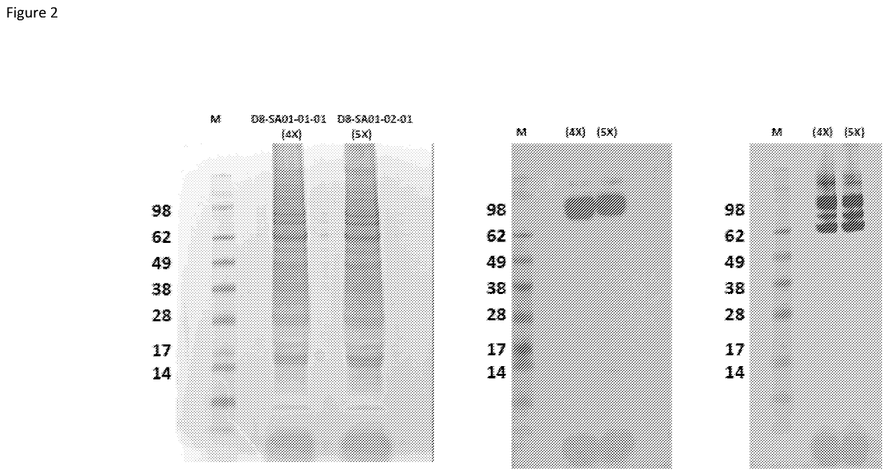

Fusion proteins of HBSAg and full-length SARS-CoV-2 spike protein (with an (EAAAK)₃ linker) was expressed recombinantly in HEK cells. The recombinant expression was carried out in two independent experiments, with the medium from 5 clones (experiment 1) and 4 clones (experiment 2) pooled and assessed for fusion protein expression as shown in FIG. 2.

The pooled medium from 5 clones (experiment 1) was designated HBSAg-(EAAAK)₃-Cov-S D8-SA01-02-01 (5×) HBSAg. The pooled medium from 4 clones (experiment 2) was designated HBSAg-(EAAAK)₃-Cov-S D8-SA01-01-01 (4×) HBSAg.

The total protein content of both fusion protein pools was determined by Bradford assay and adjusted to 1 mg/ml in a total volume of 100 ml.

Balb/c mice were immunised with either HBSAg-(EAAAK)₃-Cov-S D8-SA01-02-01 (5×) HBSAg or HBSAg-(EAAAK)₃-Cov-S D8-SA01-01-01 (4×) HBSAg, either alone or in combination with Vaxigrip influenza vaccine. The COVID-19/'flu/combination vaccines were administered either without adjuvant, with Alu-280 adjuvant or Adda-Vax adjuvant as shown in Table 3 below.

Immunisation with HBSAg-(EAAAK)₃-Cov-S D8-SA01-02-01 (5×) or HBSAg-(EAAAK)₃-Cov-S D8-SA01-01-01 (4×) was carried out using 50 μg/dose (volume 100 μl). Immunisation with the influenza vaccine was carried out using 1.5 μg/dose (volume 50 μl). Where either adjuvant was used, a 1:1 v/v vaccine:adjuvant ratio was used (totalling 100 μl for adjuvant+1 vaccine; or 150 μl for adjuvant+2 vaccines). Mice were immunised on day 0, with boosts at day 7, 14 and 28. Serum samples were obtained on day 14, and following sacrifice on day 42. The spleens of the immunised mice were also isolated for testing after sacrifice.

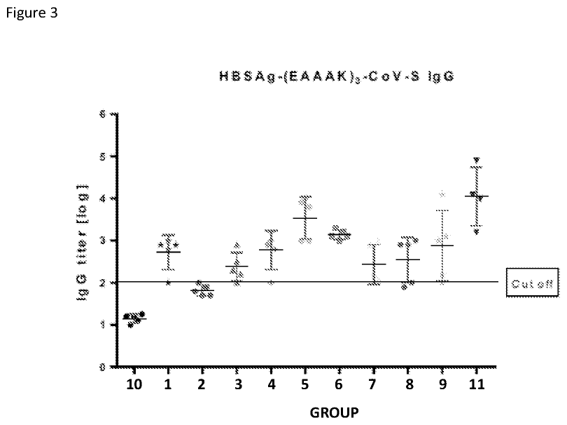

Antibody titres were measured by ELISA against the receptor binding domain (RBD) of the SARS-CoV-2 spike protein (COVID-19 antigen). As shown in FIG. 3 below, in all experimental groups (groups 1, 3-9 and 11), observable titres of anti-HBSAg-(EAAAK)₃-Cov-S IgG were present 14 days after the priming immunisation, compared with the PBS control group (group 10) or the influenza vaccine alone (group 2). Significantly, no appreciable component suppression was observed when either HBSAg-(EAAAK)₃-Cov-S fusion protein was administered with the influenza vaccine, supporting the potential clinical utility of a combined COVID-19/influenza vaccine. As FIG. 3 also shows, the use of an adjuvant, particularly Adda-Vax further increased IgG production, particularly for HBSAg-(EAAAK)₃-Cov-S D8-SA01-02-01 (5×), and the combination of HBSAg-(EAAAK)-Cov-S D8-SA01-01-01 (4×) with Vaxigrip.

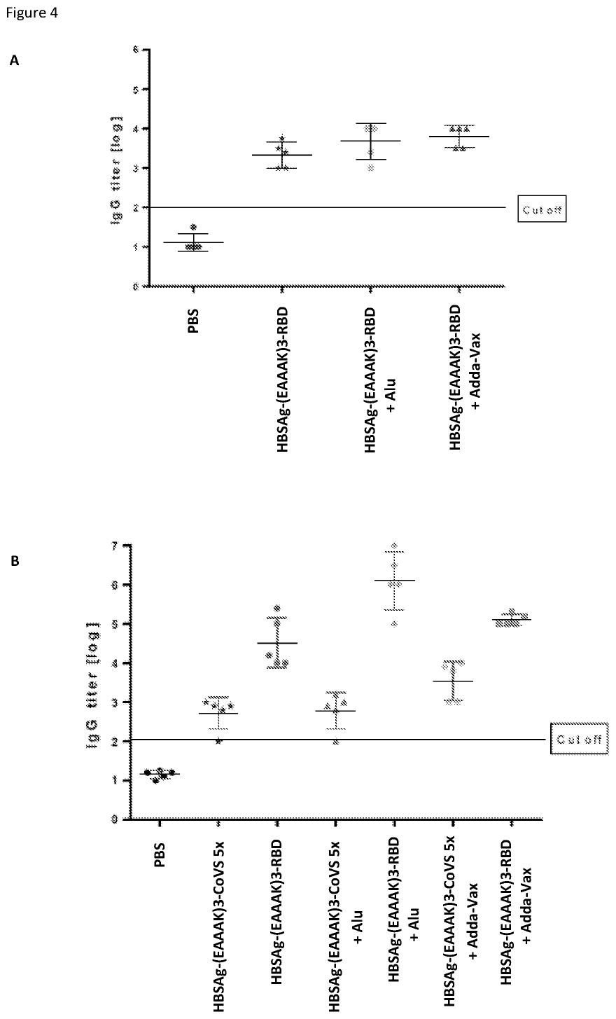

The titre of anti-COVID spike protein IgG quantified using ELISA was (alone or in combination with Vaxigrip) was compared with the IgG produced against a similar fusion protein containing only the receptor-binding domain (RBD) of the SARS-CoV-2 spike protein, HBSAg-(EAAAK)₃-Cov-S. Data for HBSAg-(EAAAK)₃-Cov-S alone is shown in FIG. 4A, and compared with HBSAg-(EAAAK)₃-Cov-S D8-SA01-02-01 (5×) in FIG. 4B. Higher titres were obtained using the RBD-fusions (FIG. 4B). Antibody titres were measured again 42 days after the priming immunisation. Again, as at day 14, in all experimental groups (groups 1, 3-9 and 11), observable titres of anti-HBSAg-(EAAAK)₃-Cov-S IgG were present 14 days after the priming immunisation, compared with the PBS control group (group 10) or the influenza vaccine alone (group 2). Significantly, no appreciable component suppression was observed when either HBSAg-(EAAAK)₃-Cov-S fusion protein was administered with the influenza vaccine, supporting the potential clinical utility of a combined COVID-19/influenza vaccine. Indeed, the anti-HBSAg-(EAAAK)₃-Cov-S IgG titre for group 3 (immunised with HBSAg-

TABLE 3
HBSAg-(EAAAK)₃-CoV-S, Influenza and HBSAg-(EAAAK)₃-CoV-S/Influenza Immunization

| | Animal | | | | |
|---|---|---|---|---|---|
| | No | | | | Injection |
| Group | Balb/c | Cage | Vaccine | Adjuvant | volume/route |
| 1 | 5 | A/B | HBSAg-(EAAAK)₃-CoV-S (HEK) D8-SA01-02-01 (5×) | None | 50 μl (i.p.) |
| 2 | 5 | C/D | Influenza (VAXIGRIP 0.5 ml) | None | 50 μl (i.p.) |
| 3 | 5 | E/F | HBSAg-(EAAAK)₃-CoV-S (HEK) D8-SA01-02-01 (5×) + | None | 100 μl (i.p.) |
| | | | Influenza (VAXIGRIP 0.5 ml) | | |
| 4 | 5 | G/H | HBSAg-(EAAAK)₃-CoV-S (HEK) D8-SA01-02-01 (5×) | Alu-280 | 100 μl (i.p.) |
| 5 | 5 | I/L | HBSAg-(EAAAK)₃-CoV-S (HEK) D8-SA01-02-01 (5×) | Adda-Vax | 100 μl (i.p.) |
| 6 | 5 | M/N | HBSAg-(EAAAK)₃-CoV-S (HEK) D8-SA01-02-01 (5×) + | Alu-280 | 150 μl (i.p.) |
| | | | Influenza (VAXIGRIP 0.5 ml) | | (50 μl + 50 μl + 50 μl) |
| 7 | 5 | O/P | HBSAg-(EAAAK)₃-CoV-S (HEK) D8-SA01-02-01 (5×) + | Adda-Vax | 150 μl (i.p.) |
| | | | Influenza (VAXIGRIP 0.5 ml) | | (50 μl + 50 μl + 50 μl) |
| 8 | 5 | Q/R | HBSAg-CoV-S (HEK) D8-SA01-01-01 (4×) | None | 50 μl (i.p.) |
| 9 | 5 | S/T | HBSAg-CoV-S (HEK) D8-SA01-01-01 (4×) + | None | 100 μl (i.p.) |
| | | | Influenza (VAXIGRIP 0.5 ml) | | |
| 10 | 5 | U/V | PBS | None | 50 μl (i.p.) |
| 11 | 4 | Z | HBSAg-CoV-S (HEK) D8-SA01-01-01 (4×) + | Adda-Vax | 150 μl (i.p.) |
| | | | Influenza (VAXIGRIP 0.5 ml) | | (50 μl + 50 μl + 50 μl) |

(EAAAK)₃-Cov-S D8-SA01-02-01 (5×) and Vaxigrip) was greater than for group 1 (immunised with HBSAg-(EAAAK)₃-Cov-S D8-SA01-02-01 (5×) alone).

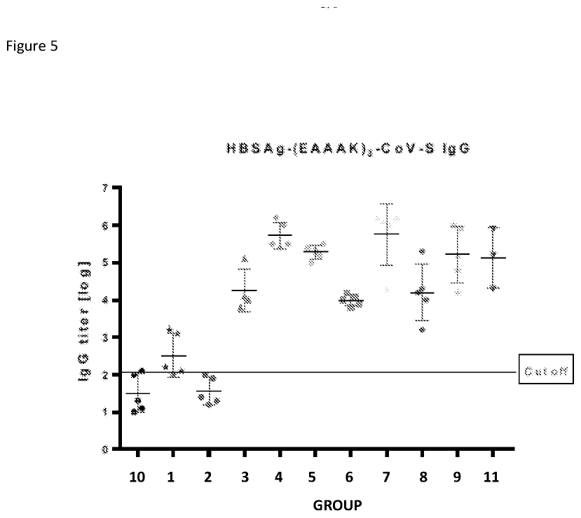

As FIG. 5 also shows, the use of an adjuvant, particularly Adda-Vax further increased IgG production, particularly for HBSAg-(EAAAK)₃-Cov-S D8-SA01-02-01 (5×) alone, or in combination with Vaxigrip.

The titre of anti-COVID spike protein IgG quantified using ELISA was (alone or in combination with Vaxigrip) was compared with the IgG produced against a similar fusion protein containing only the receptor-binding domain (RBD) of the SARS-CoV-2 spike protein, HBSAg-(EAAAK)₃-Cov-S. Data for HBSAg-(EAAAK)₃-Cov-S alone at day 42 is shown in FIG. 5A, and compared with HBSAg-(EAAAK)₃-Cov-S D8-SA01-02-01 (5×) and HBSAg-(EAAAK)₃-Cov-S D8-SA01-01-01 (4×) in FIG. 5B. The highest titres were obtained using the RBD-fusions (FIG. 58), however, high titres were maintained with HBSAg-(EAAAK)₃-Cov-S D8-SA01-01-01 (4×) in combination with Vaxigrip when formulated with Adda-Vax.

These experiments demonstrate that vaccine compositions comprising SARS-CoV-2 spike protein fusions can be successfully combined with influenza virus vaccines, with none of the expected problems of vaccine component suppression which are common in the production of combination vaccine products. Accordingly, neutralisation assays were planned using said combination vaccines.

### Example 3: Neutralisation Assay Comparing a Commercial Flu Vaccine (Vaxigrip) Alone, and a COVID-19 Vaccine (Full-Size Spike Protein Conjugated to HBSAg) Alone with a Combined Flu-Covid-19 Vaccine

The ability of SARS-CoV-2 fusion proteins, 'flu vaccine and combined COVID-19-'flu vaccines of the invention to generate neutralising antibodies against their respective antibodies can be tested using micro-neutralisation assays based on cytopathic effect (MN-CPE).

Groups of 5 Balb/c mice were vaccinated with 0.5 ml of each the above vaccines (day 0).

Serum samples were taken from the mice on day 0, 14 and 42.

1. Commercial Flu vaccine (e.g. Vaxigrip)

2. Covid-19 vaccine (e.g. HBSAg-(EAAAK)-Cov-S)

3. Combined Flu-Covid-19 vaccine

These can be repeated with or without adjuvant (e.g. Addavax)

Vero E6 cells are seeded in 96 well plates and cultured to achieve sub-confluency.

The titre of SARS-CoV-2 is calculated using a standard titration assay, and a ten-fold serial dilution (log 10) of the SARS-CoV-2 is prepared. Alternatively a 3.16-fold serial dilution (0.5 Log 10) can be carried out.

The serially diluted SARS-CoV-2 is applied to the confluent Vero cells in the 96 well plate. A column of the plate is left untreated with SARS-CoV-2 as a cell control. In addition, a sample containing known SARS-CoV-2 specific neutralising antibodies can be used as a positive control, and a human or animal depleted sample may be used as a negative control (e.g. human serum minus IgA/IgM/IgG).

After addition of the SARS-CoV-2, the plates are incubated at 37° C., 5% CO₂ for 3 days (the incubation time may be varied depending on the SARS-CoV-2 strain and variants). After incubation, the plates are observed under an inverted microscope and wells are scored as positive for SARS-CoV-2 (i.e. a CPE is observed) or negative for SARS-CoV-2 (i.e. the cells are alive and without CPE).

Once the 50% tissue culture infectious dose (TCID50) has been calculated, the MN-CPE assay can be carried out.

For the MN-CPE, Vero E6 cells are cultured and seeded in 96 well plates as before. Serum samples from the vaccinated mice are heat treated at 56 t 1° C. for 30 minutes ±10 minutes. The serum samples from the treated mice are serially diluted, first by 1:10., and then 2-fold serial dilutions are performed across the rows of the plate. The desired viral titre (one plate for SARS-CoV-2, one plate for influenza) is added to each well of the plate, following which the plates are incubated at 37±1° C., 5±1% CO₂ for 1 hour. The virus-serum mixtures are then applied to the sub-confluent pre-cultured Vero E6 cells, and the plates are incubated at 37°±1 C, 5±1 CO₂ for 3 days (the incubation time may be varied depending on the SARS-CoV-2 strain and variants).

The microneutralisation titre (MNt) is the reciprocal of the highest sample dilution that protects from CPE at least 50% of the cells. If no neutralisation is observed, it is assumed that the MNt is <10, which is under the lower limit of detection.

Serum from mice treated with HBSAg-(EAAAK)₃-Cov-S demonstrates effective neutralisation and inhibition of the CPE in Vero cells. Similarly, mice treated with the influenza vaccine produce serum with neutralisation activity against influenza. Where mice are treated with a combination of and HBSAg-(EAAAK)₃-Cov-S an influenza vaccine, neutralisation is achieved against both SARS-CoV-2 and influenza, demonstrating that there is no component suppression when using a combined SARS-CoV-2 and influenza vaccine.

The experiment is repeated using a combination of a SARS-CoV-2 RBD fragment vaccine and an influenza vaccine. Again, no component suppression is observed.

### SEQUENCE INFORMATION

SARS-CoV-2 spike protein amino acid sequence
SEQ ID NO: 1
MFVFLVLLPLVSSQCVNLTTRTQLPPAYTNSFTRGVYYPDKVFRSSVL
HSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVY
YHKNNKSWMESEFRVYSSANNCTFEYVSQPFLMDLEGKQGNFKNLREF
VFKNIDGYFKIYSKHTPINLVRDLPQGFSALEPLVDLPIGINITRFQT
LLALHRSYLTPGDSSSGWTAGAAAYYVGYLQPRTFLLKYNENGTITDA
VDCALDPLSETKCTLKSFTVEKGIYQTSNFRVQPTESIVRFPNITNLC
PFGEVFNATRFASVYAWNRKRISNCVADYSVLYNSASFSTFKCYGVSP
TKLNDLCFTNVYADSFVIRGDEVRQIAPGQTGKIADYNYKLPDDFTGC
VIAWNSNNLDSKVGGNYNYLYRLFRKSNLKPFERPISTEIYQAGSTPC
NGVEGFNCYFPLQSYGFQPTNGVGYQPYRVVVLSFELLHAPATVCGPK
KSTNLVKNKCVNFNFNGLTGTGVLTESNKKFLPFQQFGRDIADTTDAV
RDPQTLEILDITPCSFGGVSVITPGTNTSNQVAVLYQDVNCTEVPVAI
HADQLTPTWRVYSTGSNVFQTRAGCLIGAEHVNNSYECDIPIGAGICA
SYQTQTNSPRRARSVASQSIIAYTMSLGAENSVAYSNNSIAIPTNFTI
SVTTEILPVSMTKTSVDCTMYICGDSTECSNLLLQYGSFCTQLNRALT
GIAVEQDKNTQEVFAQVKQIYKTPPIKDFGGFNFSQILPDPSKPSKRS
FIEDLLFNKVTLADAGFIKQYGDCLGDIAARDLICAQKFNGLTVLPPL
LTDEMIAQYTSALLAGTITSGWTFGAGAALQIPFAMQMAYRFNGIGVT
QNVLYENQKLIANQFNSAIGKIQDSLSSTASALGKLQDVVNQNAQALN
TLVKQLSSNFGAISSVLNDILSRLDKVEAEVQIDRLITGRLQSLQTYV
TQQLIRAAEIRASANLAATKMSECVLGQSKRVDFCGKGYHLMSFPQSA
PHGVVFLHVTYVPAQEKNFTTAPAICHDGKAHFPREGVFVSNGTHWFV
TQRNFYEPQIITTDNTFVSGNCDVVIGIVNNTVYDPLQPELDSFKEEL
DKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCC
SCGSCCKFDEDDSEPVLKGVKLHYT

The RDB domain of the spike protein (residues 319 to 529) is underlined.

SARS-CoV-2 spike protein nucleic acid sequence-optimised for
expression in E. coli and containing SacI and NotI single cloning sites.
SEQ ID NO: 2 [symbol]
t ttgtttttct ggttctgctg ccgctggtta gcagccagtg tgttaatctg
accacacgta cccagctgcc tccggcatat accaatagct ttacccgtgg tgtttattat
ccggacaaag tttttcgtag cagcgttctg catagcaccc aggacctgtt tctgccgttt
tttagcaatg ttacctggtt tcatgccatt catgttagcg gcaccaatgg caccaaacgt
tttgataatc cggtgctgcc gtttaatgat ggtgtgtatt ttgcaagcac cgaaaaaagc
aacattattc gcggttggat ttttggtaca accctggata gcaaaaccca gagcctgctg
attgttaata atgccaccaa tgtggtgatc aaagtgtgcg aatttcagtt ttgcaatgat
ccgtttctgg gcgtgtatta ccacaaaaat aacaagagct ggatggaaag cgaatttcgt
gtttatagca gcgccaataa ttgcaccttt gaatatgtta gccagccgtt tctgatggat
ctggaaggta aacagggtaa ctttaaaaac ctgcgcgagt tcgtgttcaa aaacatcgat
ggttacttca aaatctatag caaacacacc ccgattaatc tggttcgtga tctgccgcag
ggttttagcg cactggaacc gctggttgat ctgccaattg gtattaacat tacccgtttt
cagaccctgc tggcactgca tcgtagctat ctgacaccgg gtgatagcag cagcggttgg
accgcaggcg cagcagcata ttatgttggt tatctgcagc ctcgtacctt tctgctgaaa
tataacgaaa atggcacaat taccgatgcc gttgattgtg ccctggatcc gctgagcgaa
accaaatgta ccctgaaaag ctttaccgtt gagaaaggta tttatcagac cagcaatttt
cgtgtgcagc cgaccgaaag cattgttcgt tttccgaata tcaccaatct gtgtccgttt
ggcgaagttt ttaatgcaac ccgttttgcc agcgtttatg catggaatcg taaacgtatt
agcaattgcg ttgccgatta tagcgttctg tataatagcg caagcttcag cacctttaaa
tgctatggtg ttagcccgac caaactgaat gatctgtgtt ttaccaatgt gtatgccgat
agctttgtga ttcgtggtga tgaagttcgt cagattgcac cgggtcagac cggtaaaatt
gcagattata actataaact gccggatgat tttacgggtt gtgttattgc ctggaatagc
aataatctgg acagcaaagt tggtggcaac tataactatc tgtatcgcct gtttcgtaag
agcaatctga aaccgtttga acgtgatatt agcaccgaga tttatcaggc aggtagcacc
gtttatagca gcgccaataa ttgcaccttt gaatatgtta gccagccgtt tctgatggat
ctggaaggta aacagggtaa ctttaaaaac ctgcgcgagt tcgtgttcaa aaacatcgat
ggttacttca aaatctatag caaacacacc ccgattaatc tggttcgtga tctgccgcag
ggttttagcg cactggaacc gctggttgat ctgccaattg gtattaacat tacccgtttt
cagaccctgc tggcactgca tcgtagctat ctgacaccgg gtgatagcag cagcggttgg
accgcaggcg cagcagcata ttatgttggt tatctgcagc ctcgtacctt tctgctgaaa
tataacgaaa atggcacaat taccgatgcc gttgattgtg ccctggatcc gctgagcgaa
accaaatgta ccctgaaaag ctttaccgtt gagaaaggta tttatcagac cagcaatttt
cgtgtgcagc cgaccgaaag cattgttcgt tttccgaata tcaccaatct gtgtccgttt
ggcgaagttt ttaatgcaac ccgttttgcc agcgtttatg catggaatcg taaacgtatt
agcaattgcg ttgccgatta tagcgttctg tataatagcg caagcttcag cacctttaaa
tgctatggtg ttagcccgac caaactgaat gatctgtgtt ttaccaatgt gtatgccgat
agctttgtga ttcgtggtga tgaagttcgt cagattgcac cgggtcagac cggtaaaatt
gcagattata actataaact gccggatgat tttacgggtt gtgttattgc ctggaatagc
aataatctgg acagcaaagt tggtggcaac tataactatc tgtatcgcct gtttcgtaag
agcaatctga aaccgtttga acgtgatatt agcaccgaga tttatcaggc aggtagcacc
ccgtgtaatg gtgttgaagg ttttaattgc tattttccgc tgcagagcta tggttttcag
ccgacaaatg gtgtgggtta tcagccgtat cgtgttgttg ttctgtcatt tgaactgctg
catgcaccgg caaccgtttg tggtccgaaa aaaagtacca atctggtgaa aaataagtgc
gtgaacttta actttaatgg tctgaccggc accggtgttc tgaccgaaag taacaaaaaa
ttcctgccgt ttcagcagtt tggccgtgat attgcagata ccaccgatgc agttcgcgat
ccgcagacac tggaaattct ggatattacc ccgtgcagct ttggtggtgt ttcagttatt
acaccgggta caaataccag caatcaggtt gcagttctgt atcaggatgt taattgtacc
gaagttccgg ttgcaattca tgcagatcag ctgaccccga cctggcgtgt gtatagcacc
ggtagcaatg tgtttcagac acgtgcaggt tgtctgattg gtgcagaaca tgtgaataat
agctatgaat gcgatattcc gattggtgcg ggtatttgtg ccagctatca gacccagacc
aatagtccgc gtcgtgcacg tagcgttgca agccagagca ttattgccta taccatgagc
ctgggtgcag aaaatagcgt tgcctatagt aataacagca ttgccattcc gaccaacttt
accattagcg ttaccaccga aattctgccg gttagcatga ccaaaaccag cgttgattgc
accatgtata tttgtggtga tagtaccgaa tgtagcaatc tgctgctgca gtatggtagc
ttttgcaccc agctgaatcg tgcactgacc ggtattgcag ttgaacagga taaaaacacg
caagaagttt ttgcacaggt caagcagatc tataaaaccc ctccgattaa agattttggc
ggtttcaatt ttagccagat cctgccggat ccgagcaaac cgagtaaacg tagctttatt
gaagatctgc tgttcaacaa agtgaccctg gcagatgcag gttttatcaa acagtatggt
gattgcctgg gcgatattgc cgcacgtgat ctgatttgtg cacagaaatt taacggcctg
accgttctgc ctccgctgct gaccgatgaa atgattgcac agtataccag cgcactgctg
gcaggcacca ttaccagtgg ttggaccttt ggtgccggtg ccgcactgca gattccgttt
gcaatgcaga tggcatatcg ttttaatggt attggtgtta cccagaacgt gctgtatgaa
aaccagaaac tgattgccaa ccagtttaat agcgccattg gcaaaattca ggatagcctg
agcagcaccg caagtgcact gggtaaactg caggacgttg ttaatcagaa tgcacaggca
ctgaataccc tggttaaaca gctgagcagt aattttggtg caatttcaag cgtgctgaac
gatattctga gccgtctgga taaagttgaa gcagaagttc agattgatcg tctgattacc
ggtcgtctgc aaagcctgca gacctatgtg acccagcagc tgattcgcgc agcagaaatt
cgtgcaagcg caaatctggc agccaccaaa atgagcgaat gtgttctggg tcagagcaaa
cgtgttgatt tttgcggcaa aggttatcac ctgatgagct ttccgcagag cgcaccgcat
ggtgttgtgt ttctgcatgt tacctatgtt ccggcacaag aaaaaaactt tacaaccgct
ccggcaattt gccatgatgg taaagcacat tttccgcgtg aaggtgtttt tgttagtaat
ggcacccatt ggtttgttac acagcgcaac ttttatgaac cgcagattat tacaaccgac
aacacctttg ttagcggtaa ctgtgatgtt gtgattggca ttgtgaataa caccgtttat
gatccactgc agccggaact ggatagcttt aaagaagaac tggacaaata tttcaaaaac
cacaccagtc cggatgttga tctgggtgat atttcaggta ttaatgccag cgtggtgaac
atccagaaag aaattgatcg cctgaatgaa gtggccaaaa atctgaatga aagcctgatt
gatctgcaag aactggggaa atatgagcag tatatcaaat ggccgtggta tatttggctg
ggttttattg caggcctgat tgcaattgtt atggtgacca ttatgctgtg ttgtatgacc
agctgttgta gctgtctgaa aggttgttgc agctgcggta gctgttgcaa atttgatgaa
gatgatagcg aaccggtgct gaaaggtgtt aaactgcatt atacctaatg a

[symbol]

The 5′ SacI single cloning site is single-underlined
The 3′ NotI single cloning site is dash-underlined
The ATG start codon is in bold and italicised
The nucleic acid sequences of SEQ ID NO: 2 translates to give the native SARS-CoV-2 spike protein of SEQ ID NO: 1

nucleic acid encoding for fusion protein HEV-SARS-Cov-2 spike protein-optimised
for expression in E. coli and containing SacI and NotI single cloning sites.
SEQ ID NO: 3
gagctcATGA TTGCACTGAC CCTGTTTAAT CTGGCAGATA CCCTGTTAGG TGGTCTGCCG
ACCGAACTGA TTAGCAGTGC CGGTGGTCAG CTGTTTTATA GCCGTCCGGT TGTTAGCGCA
AATGGTGAAC CGACCGTTAA ACTGTATACC AGCGTTGAAA ATGCACAGCA GGATAAAGGT
ATTGCAATTC CGCATGATAT TGATCTGGGT GAAAGCCGTG TTGTGATTCA GGATTATGAT
AATCAGCATG AACAGGATCG TCCGACACCG AGTCCGGCAC CGAGCCGTCC GTTTAGCGTT
CTGCGTGCAA ATGATGTTCT GTGGCTGAGC CTGACCGCAG CAGAATATGA TCAGAGCACC
TATGGTAGCA GCACCGGTCC GGTTTATGTT AGCGATAGCG TTACCCTGGT TAATGTTGCA
ACCGGTGCAC AGGCAGTTGC ACGTAGCCTG GATTGGACCA AAGTGACCCT GGATGGTCGT
CCGCTGAGCA CCATTCAGCA GTATAGCAAA ACCTTTTTTG TTCTGCCGCT GCGTGGTAAA
CTGAGCTTTT GGGAAGCAGG CACCACCAAA GCAGGTTATC CGTATAACTA TAATACCACC
GCAAGCGATC AGCTGCTGGT TGAAAACGCA GCAGGTCATC GTGTTGCAAT TAGCACCTAT
ACCACCAGTT TAGGTGCAGG TCCGGTTAGC ATTAGCGCAG TTGCAGTTCT GGCACCGCAT
AGCGCAtttg tttttctggt tctgctgccg ctggttagca gccagtgtgt taatctgacc
acacgtaccc agctgcctcc ggcatatacc aatagcttta cccgtggtgt ttattatccg
gacaaagttt ttcgtagcag cgttctgcat agcacccagg acctgtttct gccgtttttt
agcaatgtta cctggtttca tgccattcat gttagcggca ccaatggcac caaacgtttt
gataatccgg tgctgccgtt taatgatggt gtgtattttg caagcaccga aaaaagcaac
attattcgcg gttggatttt tggtacaacc ctggatagca aaacccagag cctgctgatt
gttaataatg ccaccaatgt ggtgatcaaa gtgtgcgaat ttcagttttg caatgatccg
tttctgggcg tgtattacca caaaaataac aagagctgga tggaaagcga atttcgtgtt
tatagcagcg ccaataattg cacctttgaa tatgttagcc agccgtttct gatggatctg
gaaggtaaac agggtaactt taaaaacctg cgcgagttcg tgttcaaaaa catcgatggt
tacttcaaaa tctatagcaa acacaccccg attaatctgg ttcgtgatct gccgcagggt
tttagcgcac tggaaccgct ggttgatctg ccaattggta ttaacattac ccgttttcag
accctgctgg cactgcatcg tagctatctg acaccgggtg atagcagcag cggttggacc
gcaggcgcag cagcatatta tgttggttat ctgcagcctc gtacctttct gctgaaatat
aacgaaaatg gcacaattac cgatgccgtt gattgtgccc tggatccgct gagcgaaacc
aaatgtaccc tgaaaagctt taccgttgag aaaggtattt atcagaccag caattttcgt
gtgcagccga ccgaaagcat tgttcgtttt ccgaatatca ccaatctgtg tccgtttggc
gaagttttta atgcaacccg ttttgccagc gtttatgcat ggaatcgtaa acgtattagc
aattgcgttg ccgattatag cgttctgtat aatagcgcaa gcttcagcac ctttaaatgc
tatggtgtta gcccgaccaa actgaatgat ctgtgtttta ccaatgtgta tgccgatagc
tttgtgattc gtggtgatga agttcgtcag attgcaccgg gtcagaccgg taaaattgca
gattataact ataaactgcc ggatgatttt acgggttgtg ttattgcctg gaatagcaat
aatctggaca gcaaagttgg tggcaactat aactatctgt atcgcctgtt tcgtaagagc
aatctgaaac cgtttgaacg tgatattagc accgagattt atcaggcagg tagcaccccg
tgtaatggtg ttgaaggttt taattgctat tttccgctgc agagctatgg ttttcagccg
acaaatggtg tgggttatca gccgtatcgt gttgttgttc tgtcatttga actgctgcat
gcaccggcaa ccgtttgtgg tccgaaaaaa agtaccaatc tggtgaaaaa taagtgcgtg
aactttaact ttaatggtct gaccggcacc ggtgttctga ccgaaagtaa caaaaaattc
ctgccgtttc agcagtttgg ccgtgatatt gcagatacca ccgatgcagt tcgcgatccg
cagacactgg aaattctgga tattaccccg tgcagctttg gtggtgtttc agttattaca
ccgggtacaa ataccagcaa tcaggttgca gttctgtatc aggatgttaa ttgtaccgaa
gttccggttg caattcatgc agatcagctg accccgacct ggcgtgtgta tagcaccggt
agcaatgtgt ttcagacacg tgcaggttgt ctgattggtg cagaacatgt gaataatagc
tatgaatgcg atattccgat tggtgcgggt atttgtgcca gctatcagac ccagaccaat
agtccgcgtc gtgcacgtag cgttgcaagc cagagcatta ttgcctatac catgagcctg
ggtgcagaaa atagcgttgc ctatagtaat aacagcattg ccattccgac caactttacc
attagcgtta ccaccgaaat tctgccggtt agcatgacca aaaccagcgt tgattgcacc
atgtatattt gtggtgatag taccgaatgt agcaatctgc tgctgcagta tggtagcttt
tgcacccagc tgaatcgtgc actgaccggt attgcagttg aacaggataa aaacacgcaa
gaagtttttg cacaggtcaa gcagatctat aaaacccctc cgattaaaga ttttggcggt
ttcaatttta gccagatcct gccggatccg agcaaaccga gtaaacgtag ctttattgaa
gatctgctgt tcaacaaagt gaccctggca gatgcaggtt ttatcaaaca gtatggtgat
tgcctgggcg atattgccgc acgtgatctg atttgtgcac agaaatttaa cggcctgacc
gttctgcctc cgctgctgac cgatgaaatg attgcacagt ataccagcgc actgctggca
ggcaccatta ccagtggttg gacctttggt gccggtgccg cactgcagat tccgtttgca
atgcagatgg catatcgctt taatggtatt ggtgttaccc agaacgtgct gtatgaaaac
cagaaactga ttgccaacca gtttaatagc gccattggca aaattcagga tagcctgagc
agcaccgcaa gtgcactggg taaactgcag gacgttgtta atcagaatgc acaggcactg
aataccctgg ttaaacagct gagcagtaat tttggtgcaa tttcaagcgt gctgaacgat
attctgagcc gtctggataa agttgaagca gaagttcaga ttgatcgtct gattaccggt
cgtctgcaaa gcctgcagac ctatgtgacc cagcagctga ttcgcgcagc agaaattcgt
gcaagcgcaa atctggcagc caccaaaatg agcgaatgtg ttctgggtca gagcaaacgt
gttgattttt gcggcaaagg ttatcacctg atgagctttc cgcagagcgc accgcatggt
gttgtgtttc tgcatgttac ctatgttccg gcacaagaaa aaaactttac aaccgctccg
gcaatttgcc atgatggtaa agcacatttt ccgcgtgaag gtgtttttgt tagtaatggc
acccattggt ttgttacaca gcgcaacttt tatgaaccgc agattattac aaccgacaac
acctttgtta gcggtaactg tgatgttgtg attggcattg tgaataacac cgtttatgat
ccactgcagc cggaactgga tagctttaaa gaagaactgg acaaatattt caaaaaccac
accagtccgg atgttgatct gggtgatatt tcaggtatta atgccagcgt ggtgaacatc
cagaaagaaa ttgatcgcct gaatgaagtg gccaaaaatc tgaatgaaag cctgattgat
ctgcaagaac tggggaaata tgagcagtat atcaaatggc cgtggtatat ttggctgggt
tttattgcag gcctgattgc aattgttatg gtgaccatta tgctgtgttg tatgaccagc
tgttgtagct gtctgaaagg ttgttgcagc tgcggtagct gttgcaaatt tgatgaagat
gatagcgaac cggtgctgaa aggtgttaaa ctgcattata cctaatga

[symbol]

The 5′ SacI single cloning site is single-underlined
The HEV (p239 fragment) sequence is shown in capital letters
The SARS-CoV-2 spike protein encoding sequence is shown in lower case letters
The 3′ NotI single cloning site is dash-underlined

SARS-COV-2 spike protein nucleic acid sequence-optimised for expression in
Komagataella pastoris and containing BstB1 and NotI single cloning sites.
SEQ ID NO: 4
TTCGAAacga tgttcgtgtt cttggtcctg ttgccattgg tttcttccca gtgtgttaac
ctgaccacta gaactcaatt gcctccagcc tacaccaatt ccttcaccag aggtgtttac
tacccagaca aggtgttcag atcttccgtc ttgcactcca ctcaggactt gttcttgcca
ttcttctcca acgttacctg gttccacgct attcacgttt ccggaactaa cggtactaag
agattcgaca acccagtcct gccattcaac gatggtgtct acttcgcttc taccgagaag
tccaacatca tcagaggttg gatcttcggt actaccctgg actctaagac tcagtccttg
ctgatcgtta acaacgccac caacgttgtc atcaaggttt gcgagttcca gttctgcaac
gacccattct tgggtgtgta ctaccacaag aacaacaagt cttggatgga atccgagttc
agagtttact cctccgccaa caactgtacc ttcgagtacg tttcccagcc attcttgatg
gacttggagg gtaagcaggg taacttcaag aacctgagag agttcgtttt caagaacatc
gacggttact tcaagatcta ctccaagcac accccaatca acctggttag agatttgcca
caaggtttct ccgctttgga gcctttggtt gacttgccaa tcggtatcaa catcaccaga
ttccagacct tcttggcctt gcacagatcc tacttgactc caggtgattc ttcttccggt
tggactgctg gtgctgctgc ttactatgtt ggttacttgc agccaagaac cttcctgctg
aagtacaacg agaacggaac tatcactgac gctgttgact gtgctttgga cccattgtct
gagactaagt gcaccttgaa gtccttcacc gttgagaagg gtatctacca gacctccaac
ttcagagttc agccaactga gtccatcgtc agattcccaa acatcactaa cttgtgccca
ttcggtgagg tcttcaacgc tactagattc gcttctgttt acgcctggaa cagaaagaga
atctccaact gcgttgctga ctactccgtc ttgtacaact ctgcttcatt ctccaccttc
aagtgctacg gtgtttcccc aactaagtty aacgacctgt gtttcactaa cgtctacgcc
gactccttcg ttattagagg tyacgaggtt agacagatcg ctccaggtca aactggtaag
atcgctgact acaactacaa gctgccagac gacttcaccg gttgtgttat tgcttggaac
tccaacaacc tygactccaa ggttggtggt aactacaatt acctgtaccg tctgttcaga
aagtccaact tgaagccatt cyagagagac atctccaccg agatctacca agctggttct
actccatgta acggtgtcga gggtttcaac tgctacttcc cattgcaatc ctacggtttc
caacctacca acggtgttgg ataccagcca tacagagttg tcgttttgtc cttcgagttg
ttgcacgctc cagctactgt ttgtggtcca aagaagtcca ccaacttggt caagaacaaa
tgcgtcaact ttaacttcaa cggcctgacc ggtactggtg ttttgactga atccaacaag
aagttcctgc ctttccagca gttcggtaga gacattgctg acactactga cgccgttaga
gatccacaga ctttggagat cttggacatc accccatgtt ccttcggtgg tgtttccgtt
attacccctg gaactaacac ctccaatcag gtcgctgtct tytaccagga ccttaactgt
actgaggttc cagttgctat ccacgctgac caattgactc caacttggag agtctactcc
accggttcca acgttttcca aactagagcc gcttgtttga tcggtgctga acacgtcaac
aactcctacg agtgtgacat tccaattggt gctggtatct gtgcctccta ccaaactcaa
actaactccc caagaagggc tagatccgtt gcttcccaat ccattatcgc ttacaccaty
tctttgggtg ccgagaactc tcttgcctac tctaacaact ctatcgctat ccctaccaad
ttcaccatct ccgttaccac tgagatcttg ccagtctcca tgaccaagac ttccgttgac
tgtaccatgt acatctgtgg tgactccact gagtgttcca acttgttgct gcaatacggt
tccttctgca cccagttgaa cagagctttg actggtattg ctgtcgagca agacaagaac
actcaagagg ttttcgccca ggtgaagcag atctacaaga ctccacctat taaggacttc
ggtggcttca acttctccca gattttgcca gatccatcta agccctccaa gagatccttc
attgaggacc tgctgttcaa caaggttact ttggctgacg ccggtttcat caagcagtac
ggtgattgct tgggtgacat tccagctaga gacttgatct gtgcccagaa gttcaacggt
tcgaccgttt tgccaccttt gttgaccgac gagatgatcg ctcagtacac ttctgctttg
ttggccggta ctatcacttc tcgttggaca tttggagctg gtgccgcatt gcaaattcca
ttcgctatgc aaatggccta cagattcaac ggtatcggtg ttacccagaa cytcctgtac
gagaaccaga agcttatcgc caaccagttc aactccgcta tcggtaagat tcaggactcc
ttgtcctcta ctgcttctgc cttgggaaag ttgcaggatg ttgttaacca gaatgcccag
gctttgaaca ccctggttaa gcaactgtcc tctaacttcg gtgctatctc ctccgttttg
aacgacatct tgtcccgttt ggacaaggtt gaggctgagg ttcagatcga cagattgatc
actggtagat tccagtccct gcagacttac gttactcagc agttgattag agctgccgag
attagagcct ctgctaactt ggctgctact aagatgtccg agtgtgtttt gggtcagtcc
aagagagtty acttctgcgg taagggttac cacctgatgt ctttcccaca atctgctcca
cacggtgtcg ttttcttgca cgttacttac gttccagctc aagagaagaa cttcactact
gctccagcca tttgtcacga tcgtaaggct cactttcctc gtgagggtgt tttcgtttcc
aacggtactc actggttcgt cacccagaga aacttttacg agccacagat catcaccacc
gacaacactt tcgtttctgg taactgtgac gtcgtcatcg gtatcgtgaa caacactgtc
tacgatccat tgcagccaga attggactcc ttcaaagagg aactggacaa gtactttaag
aaccacactt ccccagacgt tgacctgggt gatatttccg gtattaacgc ctccgttgtc
aacatccaaa aagagatcga ccgtttgaac gaggtcgcca agaacttgaa cgagtccttg
attgacttgc aagagctggg caagtacgag cagtacatta agtggccatg gtacatttgg
ctgggtttca ttgctggttt gatcgccatc gttatggtca ccatcatgtt gtgctgtatg
acctcctgtt gctcctgttt gaagggttgt tgttcctgcg gttcctgttg taagttcgac
gaagatgact ccgagccagt cttgaagggt gttaagttgc actacactta a

[symbol]

The 5′ BstBI single cloning site is single-underlined
The 3′ NotI single cloning site is dash-underlined
Immediately following the 5′ SacI is an ACG codon (needed for the coding sequence to be in frame with the ATG start codon, which immediately follows the ACG). These two codons are shown in bold and italicised.
The nucleic acid sequences of SEQ ID NO: 4 translates to give the native SARS-CoV-2 spike protein of SEQ ID NO: 1

nucleic acid encoding for fusion protein
HPV18L1/SARS-Cov-2 spike protein-
optimised for expression in K. pastoris
and containing BstB1 and NotI single
cloning sites.
SEQ ID NO: 5
TTCGAAacgatggctctttggagaccatccgacaacactgtttacttgcc
accaccatccgttgctagagttgttaacactgacgactacgttactagaa
cttccatcttctaccacgctggttcttccagattgttgactgttggtaac
ccatacttcagagttccagctggaggtggtaacaagcaagacatcccaaa
ggtttccgcttaccagtacagagttttcagagttcagttgccagacccaa
acaagtttggattgccagacacttccatctacaacccagagactcagaga
tgttggtttgtctggtcacccattctacaacaagttggacgacactgaat
cttctcacgctgctacttctaacgtttccgaggatgttagagacaacgtt
tccgttgactacaagcagactcagttgtgtatcttgggttgtgctccagc
tattggtgaacattgggctaagggtactgcttgtaagtccagaccattgt
ctcagggagattgtccaccattggagttgaagaacactgttttggaggac
ggtgatatggttgatactggttacggtgctatggacttctctactttgca
ggacactaagtgtgaagttccattggacatctgtcagtccatctgtaagt
acccagactacttgcaaatgtccgctgatccatacggtgactctatgttc
ttctgtttgagaagagagcagttgttcgctagacacttctggaacagagc
tggtactatgggtgacactgttccacaatccttgtacatcaagggtactg
gaatgagagcttctcctggttcttgtgtttactctccatctccatccggt
tccattgttacttccgactcccagttgttcaacaagccatactggttgca
taaggctcaaggtcacaacaacggtgtttgttggcacaaccagttgttcg
ttactgttgttgacactactagatccactaacttgactatctgtgcttcc
actcaatctccagttccaggacaatacgacgctactaagttcaagcagta
ctccagacacgttgaagagtacgacttgcagttcatcttccagttgtgta
ctatcactttgactgctgatgttatgtcctacatccactctatgaactcc
tccattttggaggattggaacttcggtgttccaccaccaccaactacttc
attggttgacacttacagattcgttcagtccgttgctatcacttgtcaaa
aggacgctgctccagctgaaaacaaggacccatacgacaagttgaagttc
tggaacgttgacttgaaagagaagttctccttggacttggaccaataccc
attgggtagaaagtttttggttcaggctggattgagaagaaagccaacta
tcggtccaagaaagagatcagctccatccgctactacttcatccaagcca
gctaagagagttagagttagagctagaaagtTCGTGTTCTTGGTCCTGTT
GCCATTGGTTTCTTCCCAGTGTGTTAACCTGACCACTAGAACTCAATTGC
CTCCAGCCTACACCAATTCCTTCACCAGAGGTGTTTACTACCCAGACAAG
GTGTTCAGATCTTCCGTCTTGCACTCCACTCAGGACTTGTTCTTGCCATT
CTTCTCCAACGTTACCTGGTTCCACGCTATTCACGTTTCCGGAACTAACG
GTACTAAGAGATTCGACAACCCAGTCCTGCCATTCAACGATGGTGTCTAC
TTCGCTTCTACCGAGAAGTCCAACATCATCAGAGGTTGGATCTTCGGTAC
TACCCTGGACTCTAAGACTCAGTCCTTGCTGATCGTTAACAACGCCACCA
ACGTTGTCATCAAGGTTTGCGAGTTCCAGTTCTGCAACGACCCATTCTTG
GGTGTGTACTACCACAAGAACAACAAGTCTTGGATGGAATCCGAGTTCAG
AGTTTACTCCTCCGCCAACAACTGTACCTTCGAGTACGTTTCCCAGCCAT
TCTTGATGGACTTGGAGGGTAAGCAGGGTAACTTCAAGAACCTGAGAGAG
TTCGTTTTCAAGAACATCGACGGTTACTTCAAGATCTACTCCAAGCACAC
CCCAATCAACCTGGTTAGAGATTTGCCACAAGGTTTCTCCGCTTTGGAGC
CTTTGGTTGACTTGCCAATCGGTATCAACATCACCAGATTCCAGACCTTG
TTGGCCTTGCACAGATCCTACTTGACTCCAGGTGATTCTTCTTCCGGTTG
GACTGCTGGTGCTGCTGCTTACTATGTTGGTTACTTGCAGCCAAGAACCT
TCCTGCTGAAGTACAACGAGAACGGAACTATCACTGACGCTGTTGACTGT
GCTTTGGACCCATTGTCTGAGACTAAGTGCACCTTGAAGTCCTTCACCGT
TGAGAAGGGTATCTACCAGACCTCCAACTTCAGAGTTCAGCCAACTGAGT
CCATCGTCAGATTCCCAAACATCACTAACTTGTGCCCATTCGGTGAGGTG
TTCAACGCTACTAGATTCGCTTCTGTTTACGCCTGGAACAGAAAGAGAAT
CTCCAACTGCGTTGCTGACTACTCCGTCTTGTACAACTCTGCTTCATTCT
CCACCTTCAAGTGCTACGGTGTTTCCCCAACTAAGTTGAACGACCTGTGT
TTCACTAACGTCTACGCCGACTCCTTCGTTATTAGAGGTGACGAGGTTAG
ACAGATCGCTCCAGGTCAAACTGGTAAGATCGCTGACTACAACTACAAGC
TGCCAGACGACTTCACCGGTTGTGTTATTGCTTGGAACTCCAACAACCTG
GACTCCAAGGTTGGTGGTAACTACAATTACCTGTACCGTCTGTTCAGAAA
GTCCAACTTGAAGCCATTCGAGAGAGACATCTCCACCGAGATCTACCAAG
CTGGTTCTACTCCATGTAACGGTGTCGAGGGTTTCAACTGCTACTTCCCA
TTGCAATCCTACGGTTTCCAACCTACCAACGGTGTTGGATACCAGCCATA
CAGAGTIGTCGTTTTGTCCTTCGAGTTGTTGCACGCTCCAGCTACTGITT
GTGGTCCAAAGAAGTCCACCAACTTGGTCAAGAACAAATGCGTCAACTTT
AACTTCAACGGCCTGACCGGTACTGGTGTTTTGACTGAATCCAACAAGAA
GTTCCTGCCTTTCCAGCAGTTCGGTAGAGACATTGCTGACACTACTGACG
CCGTTAGAGATCCACAGACTTTGGAGATCTTGGACATCACCCCATGTTCC
TTCGGTGGTGTTTCCGTTATTACCCCTGGAACTAACACCTCCAATCAGGT
CGCTGTCTTGTACCAGGACGTTAACTGTACTGAGGTTCCAGTTGCTATCC
ACGCTGACCAATTGACTCCAACTTGGAGAGTCTACTCCACCGGTTCCAAC
GTTTTCCAAACTAGAGCCGGTTGTTTGATCGGTGCTGAACACGTCAACAA
CTCCTACGAGTGTGACATTCCAATTGGTGCTGGTATCTGTGCCTCCTACC
AAACTCAAACTAACTCCCCAAGAAGGGCTAGATCCGTTGCTTCCCAATCC
ATTATCGCTTACACCATGTCTTTGGGTGCCGAGAACTCTGTTGCCTACTC
TAACAACTCTATCGCTATCCCTACCAACTTCACCATCTCCGTTACCACTG
AGATCTTGCCAGTCTCCATGACCAAGACTTCCGTTGACTGTACCATGTAC
ATCTGTGGTGACTCCACTGAGTGTTCCAACTTGTTGCTGCAATACGGTTC
CTTCTGCACCCAGTTGAACAGAGCTTTGACTGGTATTGCTGTCGAGCAAG
ACAAGAACACTCAAGAGGTTTTCGCCCAGGTGAAGCAGATCTACAAGACT
CCACCTATTAAGGACTICGGTGGCTTCAACTTCTCCCAGATTTTGCCAGA
TCCATCTAAGCCCTCCAAGAGATCCTTCATTGAGGACCTGCTGTTCAACA
AGGTTACTTTGGCTGACGCCGGTTTCATCAAGCAGTACGGTGATTGCTTG
GGTGACATTGCAGCTAGAGACTTGATCTGTGCCCAGAAGTTCAACGGTTT
GACCGTTTTGCCACCTTTGTTGACCGACGAGATGATCGCTCAGTACACTT
CTGCTTTGTTGGCCGGTACTATCACTTCTGGTTGGACATTTGGAGCTGGT
GCCGCATTGCAAATTCCATTCGCTATGCAAATGGCCTACAGATTCAACGG
TATCGGTGTTACCCAGAACGTCCTGTACGAGAACCAGAAGCTTATCGCCA
ACCAGTTCAACTCCGCTATCGGTAAGATTCAGGACTCCTTGTCCTCTACT
GCTTCTGCCTTGGGAAAGTTGCAGGATGTTGTTAACCAGAATGCCCAGGC
TTTGAACACCCTGGTTAAGCAACTGTCCTCTAACTTCGGTGCTATCTCCT
CCGTTTTGAACGACATCTTGTCCCGTTTGGACAAGGTTGAGGCTGAGGTT
CAGATCGACAGATTGATCACTGGTAGATTGCAGTCCCTGCAGACTTACGT
TACTCAGCAGTTGATTAGAGCTGCCGAGATTAGAGCCTCTGCTAACTTGG
CTGCTACTAAGATGTCCGAGTGTGTTTTGGGTCAGTCCAAGAGAGTTGAC
TTCTGCGGTAAGGGTTACCACCTGATGTCTTTCCCACAATCTGCTCCACA
CGGTGTCGTTTTCTTGCACGTTACTTACGTTCCAGCTCAAGAGAAGAACT
TCACTACTGCTCCAGCCATTTGTCACGATGGTAAGGCTCACTTTCCTCGT
GAGGGTGTTTTCGTTTCCAACGGTACTCACTGGTTCGTCACCCAGAGAAA
CTTTTACGAGCCACAGATCATCACCACCGACAACACTTTCGTTTCTGGTA
ACTGTGACGTCGTCATCGGTATCGTGAACAACACTGTCTACGATCCATTG
CAGCCAGAATTGGACTCCTTCAAAGAGGAACTGGACAAGTACTTTAAGAA
CCACACTTCCCCAGACGTTGACCTGGGTGATATTTCCGGTATTAACGCCT
CCGTTGTCAACATCCAAAAAGAGATCGACCGTTTGAACGAGGTCGCCAAG
AACTTGAACGAGTCCTTGATTGACTTGCAAGAGCTGGGCAAGTACGAGCA
GTACATTAAGTGGCCATGGTACATTTGGCTGGGTTTCATTGCTGGTTTGA
TCGCCATCGTTATGGTCACCATCATGTTGTGCTGTATGACCTCCTGTTGC
TCCTGTTTGAAGGGTTGTTGTTCCTGCGGTTCCTGTTGTAAGTTCGACGA
AGATGACTCCGAGCCAGTCTTGAAGGGTGTTAAGTTGCACTACACTTAAG

[symbol]

[symbol]

The 5′ BstBI single cloning site is single-underlined
The HPV18L1 sequence is shown in lower case letters
The SAR5-CoV-2 spike protein encoding sequence is shown in capitalised letters
The 3′ NotI single cloning site is dash-underlined
Immediately following the 5′ BstBI is an ACG codon (needed for the coding sequence to be in frame with the ATG start codon, which immediately follows the ACG). These two codons are shown in bold and italicised.

nucleic acid encoding for fusion protein
HPV16L1/SARS-Cov-2 spike protein nucleic
-optimised for expression in K. pastoris
and containing BstB1 and NotI single
cloning sites.
SEQ ID NO: 6
TTCGAAacgatgtctttgtggttgccatctgaagctactgtttacttgcc
accagttccagtttctaaagttgtttccactgacgaatacgttgctagaa
ctaacatctactaccacgctggtacttctagattgttggctgttggtcat
ccatacttcccaattaagaagccaaacaacaacaagattttggttccaaa
ggtttccggattgcaatacagagttttcagaatccatttgccagatccaa
acaagtttggtttcccagatacttctttctacaacccagacactcaaaga
tgttggtatttctggtcacccattgttgaacaagttggacgatactgaaa
acgcttctgcttacgctgctaacgctggtgttgataacagagaatgtatt
tctatggactacaagcaaactcaattgtgtttgattggttgtaagccacc
aattggtgaacattggggaaagggttctccatgtactaatgttgctgtta
accctggtgattgtccaccattggaattgattaacactgttattcaagac
ggtgatatggttgatactggtttcggtgctatggatttcactactttgca
agctaacaagtctgaagttccattggacatttgtacttccatctgtaagt
acccagactacattaagatggtttctgaaccatacggtgattctttgttc
ttctacttgagaagagaacaaatgtttgttagacacttgttcaacagagc
tggtgctgttggtgaaaacgttccagatgacttgtacattaagggttctg
gttctactgctaacttggcttcttctaactactttccaactccatctggt
tctatggttacttctgacgctcaaattttcaacaagccatactggttgca
aagagcacaaggtcataacaacggtatttgttggggtaaccaattgttcg
ttactgttgttgacactactagatccactaacatgtccttgtgtgctgct
atttctacttctgaaactacttacaagaacactaacttcaaagagtactt
gagacacggagaagaatacgacttgcaattcattttccaattgtgtaaga
ttactttgactgctgacgttatgacttacattcactctatgaactctact
attttggaagattggaacttcggattgcaaccaccaccaggtggtacttt
ggaagatacttacagattcgttacttctcaagctattgcttgtcaaaagc
atactccacctgctccaaaagaagatccattgaagaagtacactttctgg
gaagttaacttgaaagaaaagttctctgctgatttggatcaattcccatt
gggtagaaagtttttgttgcaagctggattgaaggctaaaccaaagttca
ctttgggaaagagaaaggctactccaactacttcttctacttctactact
gctaagagaaagaagagaaaattgtTCGTGTTCTTGGTCCTGTTGCCATT
GGTTTCTTCCCAGTGTGTTAACCTGACCACTAGAACTCAATTGCCTCCAG
CCTACACCAATTCCTTCACCAGAGGTGTTTACTACCCAGACAAGGTGTTC
AGATCTTCCGTCTTGCACTCCACTCAGGACTTGTTCTTGCCATTCTTCTC
CAACGTTACCTGGTTCCACGCTATTCACGTTTCCGGAACTAACGGTACTA
AGAGATTCGACAACCCAGTCCTGCCATTCAACGATGGTGTCTACTTCGCT
TCTACCGAGAAGTCCAACATCATCAGAGGTTGGATCTTCGGTACTACCCT
GGACTCTAAGACTCAGTCCTTGCTGATCGTTAACAACGCCACCAACGTTG
TCATCAAGGTTTGCGAGTTCCAGTTCTGCAACGACCCATTCTTGGGTGTG
TACTACCACAAGAACAACAAGTCTTGGATGGAATCCGAGTTCAGAGTTTA
CTCCTCCGCCAACAACTGTACCTTCGAGTACGTTTCCCAGCCATTCTTGA
TGGACTTGGAGGGTAAGCAGGGTAACTTCAAGAACCTGAGAGAGTTCGTT
TTCAAGAACATCGACGGTTACTTCAAGATCTACTCCAAGCACACCCCAAT
CAACCTGGTTAGAGATTTGCCACAAGGTTTCTCCGCTTTGGAGCCTTTGG
TTGACTTGCCAATCGGTATCAACATCACCAGATTCCAGACCTTGTTGGCC
TTGCACAGATCCTACTTGACTCCAGGTGATTCTTCTTCCGGTTGGACTGC
TGGTGCTGCTGCTTACTATGTTGGTTACTTGCAGCCAAGAACCTTCCTGC
TGAAGTACAACGAGAACGGAACTATCACTGACGCTGTTGACTGTGCTTTG
GACCCATTGTCTGAGACTAAGTGCACCTTGAAGTCCTTCACCGTTGAGAA
GGGTATCTACCAGACCTCCAACTTCAGAGTTCAGCCAACTGAGTCCATCG
TCAGATTCCCAAACATCACTAACTTGTGCCCATTCGGTGAGGTGTTCAAC
GCTACTAGATTCGCTTCTGTTTACGCCTGGAACAGAAAGAGAATCTCCAA
CTGCGTTGCTGACTACTCCGTCTTGTACAACTCTGCTTCATTCTCCACCT
TCAAGTGCTACGGTGTTTCCCCAACTAAGTTGAACGACCTGTGTTTCACT
AACGTCTACGCCGACTCCTTCGTTATTAGAGGTGACGAGGTTAGACAGAT
CGCTCCAGGTCAAACTGGTAAGATCGCTGACTACAACTACAAGCTGCCAG
ACGACTTCACCGGTTGTGTTATTGCTTGGAACTCCAACAACCTGGACTCC
AAGGTTGGTGGTAACTACAATTACCTGTACCGTCTGTTCAGAAAGTCCAA
CTTGAAGCCATTCGAGAGAGACATCTCCACCGAGATCTACCAAGCTGGTT
CTACTCCATGTAACGGTGTCGAGGGTTTCAACTGCTACTTCCCATTGCAA
TCCTACGGTTTCCAACCTACCAACGGTGTTGGATACCAGCCATACAGAGT
TGTCGTTTTGTCCTTCGAGTTGTTGCACGCTCCAGCTACTGTTTGTGGTC
CAAAGAAGTCCACCAACTTGGTCAAGAACAAATGCGTCAACTTTAACTTC
AACGGCCTGACCGGTACTGGTGTTTTGACTGAATCCAACAAGAAGTTCCT
GCCTTTCCAGCAGTTCGGTAGAGACATTGCTGACACTACTGACGCCGTTA
GAGATCCACAGACTTTGGAGATCTTGGACATCACCCCATGTTCCTTCGGT
GGTGTTTCCGTTATTACCCCTGGAACTAACACCTCCAATCAGGTCGCTGT
CTTGTACCAGGACGTTAACTGTACTGAGGTTCCAGTTGCTATCCACGCTG
ACCAATTGACTCCAACTTGGAGAGTCTACTCCACCGGTTCCAACGTTTTC
CAAACTAGAGCCGGTTGTTTGATCGGTGCTGAACACGTCAACAACTCCTA
CGAGTGTGACATTCCAATTGGTGCTGGTATCTGTGCCTCCTACCAAACTC
AAACTAACTCCCCAAGAAGGGCTAGATCCGTTGCTTCCCAATCCATTATC
GCTTACACCATGTCTTTGGGTGCCGAGAACTCTGTTGCCTACTCTAACAA
CTCTATCGCTATCCCTACCAACTTCACCATCTCCGTTACCACTGAGATCT
TGCCAGTCTCCATGACCAAGACTTCCGTTGACTGTACCATGTACATCTGT
GGTGACTCCACTGAGTGTTCCAACTTGTTGCTGCAATACGGTTCCTTCTG
CACCCAGITGAACAGAGCTTTGACTGGTATTGCTGTCGAGCAAGACAAGA
ACACTCAAGAGGTTTTCGCCCAGGTGAAGCAGATCTACAAGACTCCACCT
ATTAAGGACTTCGGTGGCTTCAACTTCTCCCAGATTTTGCCAGATCCATC
TAAGCCCTCCAAGAGATCCTTCATTGAGGACCTGCTGTTCAACAAGGTTA
CTTTGGCTGACGCCGGTTTCATCAAGCAGTACGGTGATTGCTTGGGTGAC
ATTGCAGCTAGAGACTTGATCTGTGCCCAGAAGTTCAACGGTTTGACCGT
TTTGCCACCTTTGTTGACCGACGAGATGATCGCTCAGTACACTTCTGCTT
TGTTGGCCGGTACTATCACTTCTGGTTGGACATTTGGAGCTGGTGCCGCA
TTGCAAATTCCATTCGCTATGCAAATGGCCTACAGATTCAACGGTATCGG
TGTTACCCAGAACGTCCTGTACGAGAACCAGAAGCTTATCGCCAACCAGT
TCAACTCCGCTATCGGTAAGATTCAGGACTCCTTGTCCTCTACTGCTTCT
GCCTTGGGAAAGTTGCAGGATGTTGTTAACCAGAATGCCCAGGCTTTGAA
CACCCTGGTTAAGCAACTGTCCTCTAACTTCGGTGCTATCTCCTCCGTTT
TGAACGACATCTTGTCCCGTTTGGACAAGGTTGAGGCTGAGGTTCAGATC
GACAGATTGATCACTGGTAGATTGCAGTCCCTGCAGACTTACGTTACTCA
GCAGTTGATTAGAGCTGCCGAGATTAGAGCCTCTGCTAACTTGGCTGCTA
CTAAGATGTCCGAGTGTGTTTTGGGTCAGTCCAAGAGAGTTGACTTCTGC
GGTAAGGGTTACCACCTGATGTCTTTCCCACAATCTGCTCCACACGGTGT
CGTTTTCTTGCACGTTACTTACGTTCCAGCTCAAGAGAAGAACTTCACTA
CTGCTCCAGCCATTTGTCACGATGGTAAGGCTCACTTTCCTCGTGAGGGT
GTTTTCGTTTCCAACGGTACTCACTGGTTCGTCACCCAGAGAAACTTTTA
CGAGCCACAGATCATCACCACCGACAACACTTTCGTTTCTGGTAACTGTG
ACGTCGTCATCGGTATCGTGAACAACACTGTCTACGATCCATTGCAGCCA
GAATTGGACTCCTTCAAAGAGGAACTGGACAAGTACTTTAAGAACCACAC
TTCCCCAGACGTTGACCTGGGTGATATTTCCGGTATTAACGCCTCCGTTG
TCAACATCCAAAAAGAGATCGACCGTTTGAACGAGGTCGCCAAGAACTTG
AACGAGTCCTTGATTGACTTGCAAGAGCTGGGCAAGTACGAGCAGTACAT
TAAGTGGCCATGGTACATTTGGCTGGGTTTCATTGCTGGTTTGATCGCCA
TCGTTATGGTCACCATCATGTTGTGCTGTATGACCTCCTGTTGCTCCTGT
TTGAAGGGTTGTTGTTCCTGCGGTTCCTGTTGTAAGTTCGACGAAGATGA
CTCCGAGCCAGTCTTGAAGGGTGTTAAGTTGCACTACACTTAAGCGGCCG [symbol]
C

The 5′ BstBI single cloning site is single-underlined
The HPV16L1 sequence is shown in lower case letters
The SARS-CoV-2 spike protein encoding sequence is shown in capitalised letters
The 3′ NotI single cloning site is dash-underlined
Immediately following the 5′ BstBI is an ACG codon (needed for the coding sequence to be in frame with the ATG start codon, which immediately follows the ACG). These two codons are shown in bold and italicised.

SARS-COV-2 spike protein nucleic acid sequence-optimised for expression
in humans (293F) and containing NheI and NotI single cloning sites.
SEQ ID NO: 7
GCTAGCgaca tgttcgtgtt tctggtgctg ctgcctctgg tgtccagcca gtgtgtgaac
ctgaccacca gaacacagct gcctccagcc tacaccaata gcttcaccag gggcgtgtac
taccccgaca aggtgttcag atctagcgtg ctgcacagca cccaggacct gtttctgccc
ttcttcagca acgtgacctg gttccacgcc atccacgtgt ccggcaccaa tcgcaccaag
agattcgaca accccgtgct gcccttcaac gatggggtgt actttgccag caccgagaag
tccaacatca tcagaggctg gatcttcggc accacactgg acagcaagac ccagagcctg
ctgatcgtga acaacgccac caacgtggtc atcaaagtgt gcgagttcca gttctgcaac
gacccattcc tgggagtcta ctaccacaag aacaacaaga gctggatgga aagcgagttc
cgggtgtaca gcagcgccaa caactgcacc ttcgagtacy tgtcccagcc tttcctgatg
gacctggaag gcaagcaggg caacttcaag aacctgcgcg agttcgtgtt caagaacatc
gacggctact tcaagatcta cagcaagcac acccctatca acctcgtgcg ggatctgcct
cagggctttt ctgctctgga acctctggtg gacctgccta tcggcatcaa catcacccgg
tttcagaccc tgctggccct gcacagatct tacctgacac ctggcgatag cagctctgga
tggacagctg gcgccgctgc ctattatgty ggctacctgc agcctcggac cttcctgctg
aagtacaacg agaacggcac catcaccgac gccgtggatt gtgctctgga tcccctgagc
gagacaaagt gcaccctgaa gtccttcacc gtggaaaagg gcatctacca gaccagcaac
ttcagagtgc agcccaccga gagcatcgtg ccgttcccca atatcaccaa tctgtgcccc
ttcggcgagg tcttcaatgc cacaagattt gccagcgtgt acgcctggaa ccggaagaga
atcagcaact gcgtggccga ctacagcgtg ctgtacaata gcgccagctt cagcaccttc
aagcgctacg gcgtgtcccc taccaagctg aacgacctgt gcttcaccaa tgtgtacgcc
gacagcttcg tgatcagagg cgacgaagtt cggcagatcg ctcctggaca gacaggcaag
atcgccgatt acaactacaa gctgcccgac gacttcaccg gctgcgtgat cgcctggaat
agcaacaacc tggactccaa agtcggcggc aactacaact acctgtaccg gctgttccgg
aagtccaatc tgaagccctt cgagcgggac atctccaccg aaatctatca ggccggcagc
accccttgta acggcgtgga aggcttcaac tcctacttcc cactgcagtc ctacggcttt
cagcctacca atggcgtggg ctatcagccc tatagagtgg tggtgctgag cttcgaactg
ctgcatgccc ctgctaccgt gtgcggccct aagaagtcta ccaacctggt caagaacaaa
tgcgtgaact tcaacttcaa cggcctgacc ggcacaggcg tgctgacaga gagcaacaag
aagttcctgc ctttccagca gtttggccgg gatatcgccg ataccacaga cgccgttaga
gatccccaga cactggaaat cctggacatc accccatgca gctttggcgg agtgtctgtg
atcaccccty gcaccaatac cagcaatcag gtggccgtgc tgtatcagga cgtgaactgt
acagaggtgc ccgtggccat tcacgccgat caactgacac ccacttggag agtgtactcc
accggctcca acgtgttcca gactagagcc ggatgtctga tcggagccga gcacgtgaac
aatagctacg agtgcgacat ccccatcggc gctggcatct gcgccagcta ccagacacag
acaaatagcc ccagacgggc cagaagcgty gcctctcaga gcatcattgc ctacacaaty
agcctgggcg ccgagaattc tctggcctac agcaacaact ctatcgctat ccccaccaac
ttcaccatca gcgtgaccac cgagatcctg cctgtgtcca tgaccaagac cagcgtggad
tgcaccatgt acatctgcgg cgattccacc gagtgcagca acctgctgct gcagtacggc
agcttctgca cccagctgaa tagagccctg acagggatcg ccgtygaaca ggacaagaac
acccaagagg tcttcgccca agtgaagcag atctacaaga cccctcctat caaggacttc
ggcggcttca atttcagcca gattctgccc gatcctagca agcccagcaa gcggagcttt
atcgaggacc tgctgttcaa caaagtgaca ctggccgacg ccggcttcat caagcagtat
ggcgattgcc tgggcgacat tgccgccaga gatctgattt gcgcccagaa gtttaacgga
ctgacagtgc tgcctcctct gctgaccgat gagatgatcg cccagtacac atctgctctg
ctggccggca caatcaccag cggatggaca tttggagctg gcgcagccct gcagatcccc
tttgctatgc agatggccta ccggttcaac ggcatcggag tgacccagaa tgtgctgtac
gagaaccaga agctgatcgc caaccagttc aacagcgcca tcggcaagat ccaggatagc
ctgtctagca cagccagcgc tctgggcaaa ctgcaggacg tggtcaatca gaacgctcag
gccctgaaca ccctcgtgaa gcagctgagc agcaatttcg gcgccatcag ctccgtgctg
aacgatatcc tgagccggct ggataaggty gaagccgagg tgcagatcga cagactgatc
acaggcagac tgcagagcct ccagacatac gtgacccagc agctgatcag agccgccgag
attagagcct ctgccaatct ggccgccacc aagatgtctg agtgtgtgct gggccagagc
aagagagtgg atttctgcgg caagggctac cacctgatga gctttccaca gtctgctcct
cacggcgtgg tgtttctgca cgtgacctat gtgcccgctc aagagaagaa cttcacaaca
gcccctgcca tctgccacga cggaaaggcc cattttccta gagaaggcgt gttcgtgtcc
aacggcaccc attggttcgt gacacagcgg aacttctacg agccccagat catcaccacc
gacaacacct tcgtgtctgg caactgtgac gtcgtgatcg gcattgtgaa caacaccgtg
tacgaccctc tccagcccga gctggacagc ttcaaagagg aactggacaa gtactttaag
aaccacacaa gccccgacgt ggacctgggc gatattagcg gcatcaatgc ctccgtggtc
aacatccaga aagagatcga ccggctgaac gaggtggcca agaatctgaa cgagagcctg
atcgacctgc aagaactggg gaagtacgag cagtacatca agtggccctg gtacatctgg
ctgggcttta tcgccggact gattgccatc gtgatggtca caatcatgct gtgctgcatg
accagctgct gtagctgcct gaagggctgt tgcagctgtg gcagctgctg caagttcgac
gaggatgata gcgagcctgt gctgaagggc gtgaaactgc actacacc

[symbol]

The 5′ NheI single cloning site is single-underlined
The 3′ NotI single cloning site is dash-underlined
Immediately following the 5′ NheI is an GAC codon (needed for the coding sequence to be in frame with the ATG start codon, which immediately follows the GAC). These two codons are shown in bold and italicised.
The nucleic acid sequences of SEQ ID NO: 7 translates to give the native SARS-CoV-2 spike protein of SEQ ID NO: 1

nucleic acid encoding for fusion protein
HBSAg/SARS-COV-2 spike protein-
optimised for expression in humans (293F)
and containing NheI and NotI single
cloning sites.
SEQ ID NO: 8
GCTAGCGACatgaactttctgggcggtacgacagtatgccttggacaaa
attcacaatctccgacgtctaatcactcccctacaagttgtccaccgac
ttgccccggctataggtggatgtgtctcagacgattcataatctttctc
ttccctggttcatccactacatctacgggtccctgtagaacatgcacca
cacctgcacagggcacctccatgtatccgtcatgctgctgcacgaaacc
atcagatggtaactgcacgtgcataccgatcccctcatcatgggcgttt
gggaaatttctgtgggagtgggcctcagcccggttttccTTCGTGTTTC
TGGTGCTGCTGCCTCTGGTGTCCAGCCAGTGTGTGAACCTGACCACCAG
AACACAGCTGCCTCCAGCCTACACCAATAGCTTCACCAGGGGCGTGTAC
TACCCCGACAAGGTGTTCAGATCTAGCGTGCTGCACAGCACCCAGGACC
TGTTTCTGCCCTTCTTCAGCAACGTGACCTGGTTCCACGCCATCCACGT
GTCCGGCACCAATGGCACCAAGAGATTCGACAACCCCGTGCTGCCCTTC
AACGATGGGGTGTACTTTGCCAGCACCGAGAAGTCCAACATCATCAGAG
GCTGGATCTTCGGCACCACACTGGACAGCAAGACCCAGAGCCTGCTGAT
CGTGAACAACGCCACCAACGTGGTCATCAAAGTGTGCGAGTTCCAGTTC
TGCAACGACCCATTCCTGGGAGTCTACTACCACAAGAACAACAAGAGCT
GGATGGAAAGCGAGTTCCGGGTGTACAGCAGCGCCAACAACTGCACCTT
CGAGTACGTGTCCCAGCCTTTCCTGATGGACCTGGAAGGCAAGCAGGGC
AACTTCAAGAACCTGCGCGAGTTCGTGTTCAAGAACATCGACGGCTACT
TCAAGATCTACAGCAAGCACACCCCTATCAACCTCGTGCGGGATCTGCC
TCAGGGCTTTTCTGCTCTGGAACCTCTGGTGGACCTGCCTATCGGCATC
AACATCACCCGGTTTCAGACCCTGCTGGCCCTGCACAGATCTTACCTGA
CACCTGGCGATAGCAGCTCTGGATGGACAGCTGGCGCCGCTGCCTATTA
TGTGGGCTACCTGCAGCCTCGGACCTTCCTGCTGAAGTACAACGAGAAC
GGCACCATCACCGACGCCGTGGATTGTGCTCTGGATCCCCTGAGCGAGA
CAAAGTGCACCCTGAAGTCCTTCACCGTGGAAAAGGGCATCTACCAGAC
CAGCAACTTCAGAGTGCAGCCCACCGAGAGCATCGTGCGGTTCCCCAAT
ATCACCAATCTGTGCCCCTTCGGCGAGGTGTTCAATGCCACAAGATTTG
CCAGCGTGTACGCCTGGAACCGGAAGAGAATCAGCAACTGCGTGGCCGA
CTACAGCGTGCTGTACAATAGCGCCAGCTTCAGCACCTTCAAGTGCTAC
GGCGTGTCCCCTACCAAGCTGAACGACCTGTGCTTCACCAATGTGTACG
CCGACAGCTTCGTGATCAGAGGCGACGAAGTTCGGCAGATCGCTCCTGG
ACAGACAGGCAAGATCGCCGATTACAACTACAAGCTGCCCGACGACTTC
ACCGGCTGCGTGATCGCCTGGAATAGCAACAACCTGGACTCCAAAGTCG
GCGGCAACTACAACTACCTGTACCGGCTGTTCCGGAAGTCCAATCTGAA
GCCCTTCGAGCGGGACATCTCCACCGAAATCTATCAGGCCGGCAGCACC
CCTTGTAACGGCGTGGAAGGCTTCAACTGCTACTTCCCACTGCAGTCCT
ACGGCTTTCAGCCTACCAATGGCGTGGGCTATCAGCCCTATAGAGTGGT
GGTGCTGAGCTTCGAACTGCTGCATGCCCCTGCTACCGTGTGCGGCCCT
AAGAAGTCTACCAACCTGGTCAAGAACAAATGCGTGAACTTCAACTTCA
ACGGCCTGACCGGCACAGGCGTGCTGACAGAGAGCAACAAGAAGTTCCT
GCCTTTCCAGCAGTTTGGCCGGGATATCGCCGATACCACAGACGCCGTT
AGAGATCCCCAGACACTGGAAATCCTGGACATCACCCCATGCAGCTTTG
GCGGAGTGTCTGTGATCACCCCTGGCACCAATACCAGCAATCAGGTGGC
CGTGCTGTATCAGGACGTGAACTGTACAGAGGTGCCCGTGGCCATTCAC
GCCGATCAACTGACACCCACTTGGAGAGTGTACTCCACCGGCTCCAACG
TGTTCCAGACTAGAGCCGGATGTCTGATCGGAGCCGAGCACGTGAACAA
TAGCTACGAGTGCGACATCCCCATCGGCGCTGGCATCTGTGCCAGCTAC
CAGACACAGACAAATAGCCCCAGACGGGCCAGAAGCGTGGCCTCTCAGA
GCATCATTGCCTACACAATGAGCCTGGGCGCCGAGAATTCTGTGGCCTA
CAGCAACAACTCTATCGCTATCCCCACCAACTTCACCATCAGCGTGACC
ACCGAGATCCTGCCTGTGTCCATGACCAAGACCAGCGTGGACTGCACCA
TGTACATCTGCGGCGATTCCACCGAGTGCAGCAACCTGCTGCTGCAGTA
CGGCAGCTTCTGCACCCAGCTGAATAGAGCCCTGACAGGGATCGCCGTG
GAACAGGACAAGAACACCCAAGAGGTGTTCGCCCAAGTGAAGCAGATCT
ACAAGACCCCTCCTATCAAGGACTTCGGCGGCTTCAATTTCAGCCAGAT
TCTGCCCGATCCTAGCAAGCCCAGCAAGCGGAGCTTTATCGAGGACCTG
CTGTTCAACAAAGTGACACTGGCCGACGCCGGCTTCATCAAGCAGTATG
GCGATTGCCTGGGCGACATTGCCGCCAGAGATCTGATTTGCGCCCAGAA
GTTTAACGGACTGACAGTGCTGCCTCCTCTGCTGACCGATGAGATGATC
GCCCAGTACACATCTGCTCTGCTGGCCGGCACAATCACCAGCGGATGGA
CATTTGGAGCTGGCGCAGCCCTGCAGATCCCCTTTGCTATGCAGATGGC
CTACCGGTTCAACGGCATCGGAGTGACCCAGAATGTGCTGTACGAGAAC
CAGAAGCTGATCGCCAACCAGTTCAACAGCGCCATCGGCAAGATCCAGG
ATAGCCTGTCTAGCACAGCCAGCGCTCTGGGCAAACTGCAGGACGTGGT
CAATCAGAACGCTCAGGCCCTGAACACCCTCGTGAAGCAGCTGAGCAGC
AATTTCGGCGCCATCAGCTCCGTGCTGAACGATATCCTGAGCCGGCTGG
ATAAGGTGGAAGCCGAGGTGCAGATCGACAGACTGATCACAGGCAGACT
GCAGAGCCTCCAGACATACGTGACCCAGCAGCTGATCAGAGCCGCCGAG
ATTAGAGCCTCTGCCAATCTGGCCGCCACCAAGATGTCTGAGTGTGTGC
TGGGCCAGAGCAAGAGAGTGACCTATGTGCCCGCTCAAGAGAAGAACTT
CACAACAGCCCCTGCCATCTGCCACGACGGAAAGGCCCATTTTCCTAGA
GAAGGCGTGTTCGTGTCCAACGGCACCCATTGGTTCGTGACACAGCGGA
ACTTCTACGAGCCCCAGATCATCACCACCGACAACACCTTCGTGTCTGG
CAACTGTGACGTCGTGATCGGCATTGTGAACAACACCGTGTACGACCCT
CTGCAGCCCGAGCTGGACAGCTTCAAAGAGGAACTGGACAAGTACTTTA
AGAACCACACAAGCCCCGACGTGGACCTGGGCGATATTAGCGGCATCAA
TGCCTCCGTGGTCAACATCCAGAAAGAGATCGACCGGCTGAACGAGGTG
GCCAAGAATCTGAACGAGAGCCTGATCGACCTGCAAGAACTGGGGAAGT
ACGAGCAGTACATCAAGTGGCCCTGGTACATCTGGCTGGGCTTTATCGC
CGGACTGATTGCCATCGTGATGGTCACAATCATGCTGTGCTGCATGACC
AGCTGCTGTAGCTGCCTGAAGGGCTGTTGCAGCTGTGGCAGCTGCTGCA
AGTTCGACGAGGATGATAGCGAGCCTGTGCTGAAGGGCGTGAAACTGCA
CTACACC

[symbol]

The 5′ NheI single cloning site is single-underlined
The HSBAg sequence is shown in lower case letters
The SARS-CoV-2 spike protein encoding sequence is shown in capitalised letters
The 3′ NotI single cloning site is dash-underlined
Immediately following the 5′ NheI is an GAC codon (needed for the coding sequence to be in frame with the ATG start codon, which immediately follows the GAC). These two codons are shown in bold and italicised.

amino acid sequence corresponding to
SEQ ID NO: 3 (fusion protein HEV-SARS-COV-2
spike protein-optimised for expression in
E. coli and containing
SacI and NotI single cloning sites.)
SEQ ID NO: 9
MIALTLENLADTLLGGLPTELISSAGGQLFYSRPVV
SANGEPTVKLYTSVENAQQDKGIAIPHDIDLGESR
VVIQDYDNQHEQDRPTPSPAPSRPFSVLRANDVLW
LSLTAAEYDQSTYGSSTGPVYVSDSVTLVNVATGA
QAVARSLDWTKVTLDGRPLSTIQQYSKTFFVLPLR
GKLSFWEAGTTKAGYPYNYNTTASDQLLVENAAGH
RVAISTYTTSLGAGPVSISAVAVLAPHSAFVFLVL
LPLVSSQCVNLTTRTQLPPAYTNSFTRGVYYPDKV
FRSSVLHSTQDLFLPFFSNVTWFHAIHVSGTNGTK
RFDNPVLPENDGVYFASTEKSNIIRGWIFGTTLDS
KTQSLLIVNNATNVVIKVCEFQFCNDPFLGVYYHK
NNKSWMESEFRVYSSANNCTFEYVSQPFLMDLEGK
QGNFKNLREFVFKNIDGYFKIYSKHTPINLVRDLP
QGFSALEPLVDLPIGINITRFQTLLALHRSYLTPG
DSSSGWTAGAAAYYVGYLQPRTELLKYNENGTITN
SNNLDSKVGGNYNYLYRLFRKSNLKPFERDISTEI
YQAGSTPCNGVEGENCYFPLQSYGFQPTNGVGYQP
YRVVVLSFELLHAPATVCGPKKSTNLVKNKCVNFN
FNGLTGTGVLTESNKKFLPFQQFGRDIADTTDAVR
DPQTLEILDITPCSFGGVSVITPGTNTSNQVAVLY
QDVNCTEVPVAIHADQLTPTWRVYSTGSNVFQTRA
GCLIGAEHVNNSYECDIPIGAGICASYQTQTNSPR
RARSVASQSIIAYTMSLGAENSVAYSNNSIAIPTN
FTISVTTEILPVSMTKTSVPSKRSFIEDLLFNKVT
LADAGFIKQYGDCLGDIAARDLICAQKENGLTVLP
PLLTDEMIAQYTSALLAGTITSGWTEGAGAALQIP
FAMQMAYRENGIGVTQNVLYENQKLIANQFNSAIG
KIQDSLSSTASALGKLQDVVNQNAQALNTLVKQLS
SNEGAISSVLNDILSRLDKVEAEVQIDRLITGRLQ
SLQTYVTQQLIRAAEIRASANLAATKMSECVLGQS
KRVDFCGKGYHLMSFPQSAPHGVVFLHVTYVPAQE
KNFTTAPAICHDGKAHFPREGVFVSNGTHWEVTQR
NFYEPQIITTDNTFVSGNCDVVIGIVNNTVYDPLQ
PELDSFKEELDKYFKNHTSPDVDLGDISGINASVV
NIQKEIDRLNEVAKNLNESLIDLQELGKYEQYIKW
PWYIWLGFIAGLIAIVMVTIMLCCMTSCCSCLKGC
CSCGSCCKFDEDDSEPVLKGVKLHYT

amino acid sequence corresponding to
SEQ ID NO: 5 (fusion protein HPV18L1/SARS-
COV-2 spike protein-optimised for expression
in K. pastoris and containing BstB1 and
NotI single cloning sites.)
SEQ ID NO: 10
MALWRPSDNTVYLPPPSVARVVNTDDYVTRTSIFYH
AGSSRLLTVGNPYFRVPAGGGNKQDIPKVSAYQYR
VFRVQLPDPNKFGLPDTSIYNPETQRLVWACAGVE
IGRGQPLGVGLSGHPFYNKLDDTESSHAATSNVSE
DVRDNVSVDYKQTQLCILGCAPAIGEHWAKGTACK
SRPLSQGDCPPLELKNTVLEDGDMVDTGYGAMDFS
TLQDTKCEVPLDICQSICKYPDYLQMSADPYGDSM
FFCLRREQLFARHEWNRAGTMGDTVPQSLYIKGTG
MRASPGSCVYSPSPSGSIVTSDSQLFNKPYWLHKA
QGHNNGVCWHNQLEVTVVDTTRSTNLTICASTQSP
VPGQYDATKFKQYSRHVEEYDLQFIFQLCTITLTA
DVMSYIHSMNSSILEDWNFGVPPPPTTSLVDTYRE
VQSVAITCQKDAAPAENKDPYDKLKFWNVDLKEKF
SLDLDQYPLGRKFLVQAGLRRKPTIGPRKRSAPSA
TTSSKPAKRVRVRARKEVFLVLLPLVSSQCVNLTT
RTQLPPAYTNSFTRGVYYPDKVFRSSVLHSTQDLF
LPFFSNVTWFHAIHVSGTNGTKRFDNPVLPFNDGV
YFASTEKSNIIRGWIFGTTLDSKTQSLLIVNNATN
VVIKVCEFQFCNDPFLGVYYHKNNKSWMESEFRVY
SSANNCTFEVQPTESIVRFPNITNLCPFGEVENAT
RFASVYAWNRKRISNCVADYSVLYNSASFSTEKCY
GVSPTKLNDLCFTNVYADSEVIRGDEVRQIAPGQT
GKIADYNYKLPDDFTGCVIAWNSNNLDSKVGGNYN
YLYRLFRKSNLKPFERDISTEIYQAGSTPCNGVEG
ENCYFPLQSYGFQPTNGVGYQPYRVVVLSFELLHA
PATVCGPKKSTNLVKNKCVNFNENGLTGTGVLTES
NKKELPFQQFGRDIADTTDAVRDPQTLEILDITPC
SFGGVSVITPGTNTSNQVAVLYQDVNCTEVPVAIH
ADQLTPTWRVYSTGSNVFQTRAGCLIGAEHVNNSY
ECDIPIGAGICASYQTQTNSPRRARSVASQSIIAY
TMSLGAENSVAYSNNSIAIPTNFTISVTTEILPVS
MTKTSVDCTMYICGDSTECSNLLLQYGSFCTQLNR
ALTGIAVEQDKNTQEVFAQVKQIYKTPPIKDFGGF
NFSQILPDPSKPSKRSFIEDLLFNKVTLADAGFIK
QYGDCLGDIAARDLICAQKENGLTVLPPLLTDEMI
AQYTSALLAGTITSGWTFGAGAALQIPFAMQMAYR
ENGIGVTQNVLYENRLITGRLQSLQTYVTQQLIRA
AEIRASANLAATKMSECVLGQSKRVDFCGKGYHLM
SFPQSAPHGVVFLHVTYVPAQEKNETTAPAICHDG
KAHFPREGVEVSNGTHWEVTQRNFYEPQIITTDNT
FVSGNCDVVIGIVNNTVYDPLQPELDSFKEELDKY
FKNHTSPDVDLGDISGINASVVNIQKEIDRLNEVA
KNLNESLIDLQELGKYEQYIKWPWYIWLGFIAGLI
AIVMVTIMLCCMTSCCSCLKGCCSCGSCCKFDEDD
SEPVLKGVKLHYT

amino acid sequence corresponding to
SEQ ID NO: 6 (fusion protein HPV16L1/
SARS-COV-2 spike protein nucleic-
optimised for expression in
K. pastoris and containing BstB1
and NotI single cloning sites.)
SEQ ID NO: 11
MSLWLPSEATVYLPPVPVSKVVSTDEYVARTNIYY
HAGTSRLLAVGHPYFPIKKPNNNKILVPKVSGLQY
RVFRIHLPDPNKFGFPDTSFYNPDTQRLVWACVGV
EVGRGQPLGVGISGHPLLNKLDDTENASAYAANAG
VDNRECISMDYKQTQLCLIGCKPPIGEHWGKGSPC
TNVAVNPGDCPPLELINTVIQDGDMVDTGFGAMDF
TTLQANKSEVPLDICTSICKYPDYIKMVSEPYGDS
LFFYLRREQMFVRHLFNRAGAVGENVPDDLYIKGS
GSTANLASSNYFPTPSGSMVTSDAQIFNKPYWLQR
AQGHNNGICWGNQLFVTVVDTTRSTNMSLCAAIST
SETTYKNTNFKEYLRHGEEYDLQFIFQLCKITLTA
DVMTYIHSMNSTILEDWNFGLQPPPGGTLEDTYRF
VTSQAIACQKHTPPAPKEDPLKKYTFWEVNLKEKF
SADLDQFPLGRKFLLQAGLKAKPKFTLGKRKATPT
TSSTSTTAKRKKRKLFVFLVLLPLVSSQCVNLTTR
TQLPPAYTNSFTRGVYYPDKVFRSSVLHSTQDLFL
PFFSNVTWFHAIHVSGTNGTKRFDNPVLPENDGVY
FASTEKSNIIRGWIFGTTLDSKTQSLLIVNNATNV
VIKVCEFQFCNDPFLGVYYHKNNKSWMESEFRVYS
SANNCTFEYVSQPFLMDLEGKQGNFKNLREFVFKN
IDGYFKIYSKHTPINLVRDLPQGFSALEPLVDLPI
GINITRFQTLLALHRSYLTPGDSSSGWTAGAAAYY
VGYLQPRTFLLKYNENGTITDAVDCALDPLSETKC
TLKSFTVEKGIYQTSNFRVQPTESIVRFPNITNLC
PFGEVENATRFASVYAWNRKRISNCVADYSVLYNS
ASFSTFKCYGVSPTKLNDLCFTNVYADSFVIRGDE
VRQIAPGQTGKIADYNYKLPDDFTGCVIAWNSNNL
DSKVGGNYNYLYRLERKSNLKPFERDISTETGTGV
LTESNKKELPFQQFGRDIADTTDAVRDPQTLEILD
ITPCSFGGVSVITPGTNTSNQVAVLYQDVNCTEVP
VAIHADQLTPTWRVYSTGSNVFQTRAGCLIGAEHV
NNSYECDIPIGAGICASYQTQTNSPRRARSVASQS
IIAYTMSLGAENSVAYSNNSIAIPTNETISVTTEI
LPVSMTKTSVDCTMYICGDSTECSNLLLQYGSFCT
QLNRALTGIAVEQDKNTQEVFAQVKQIYKTPPIKD
FGGFNFSQILPDPSKPSKRSFIEDLLENKVTLADA
GFIKQYGDCLGDIAARDLICAQKENGLTVLPPLLT
DEMIAQYTSALLAGTITSGWTFGAGAALQIPFAMQ
MAYRENGIGVTQNVLYENQKITGRLQSLQTYVTQQ
LIRAAEIRASANLAATKMSECVLGQSKRVDFCGKG
YHLMSFPQSAPHGVVFLHVTYVPAQEKNFTTAPAI
CHDGKAHFPREGVEVSNGTHWFVTQRNFYEPQIIT
TDNTFVSGNCDVVIGIVNNTVYDPLQPELDSFKEE
LDKYFKNHTSPDVDLGDISGINASVVNIQKEIDRL
NEVAKNLNESLIDLQELGKYEQYIKWPWYIWLGFI
AGLIAIVMVTIMLCCMTSCCSCLKGCCSCGSCCKF
DEDDSEPVLKGVKLHYT

amino acid sequence corresponding to
SEQ ID NO: 8 (fusion protein HBSAg/SARS-
COV-2 spike protein-optimised for
expression in humans (293F) and
containing NheI and NotI single
cloning sites.)
SEQ ID NO: 12
MNFLGGTTVCLGQNSQSPTSNHSPTSCPPTCPGYR
WMCLRRFIIFLFILLLCLIFLLVLLDYQGMLPVCP
LIPGSSTTSTGPCRTCTTPAQGTSMYPSCCCTKPS
DGNCTCIPIPSSWAFGKFLWEWASARFSFVFLVLL
PLVSSQCVNLTTRTQLPPAYTNSFTRGVYYPDKVE
RSSVLHSTQDLFLPFFSNVTWFHAIHVSGTNGTKR
FDNPVLPFNDGVYFASTEKSNIIRGWIFGTTLDSK
TQSLLIVNNATNVVIKVCEFQFCNDPFLGVYYHKN
NKSWMESEFRVYSSANNCTFEYVSQPFLMDLEGKQ
GNFKNLREFVFKNIDGYFKIYSKHTPINLVRDLPQ
GFSALEPLVDLPIGINITRFQTLLALHRSYLTPGD
SSSGWTAGAAAYYVGYLQPRTELLKYNENGTITDA
VDCALDPLSETKCTLKSFTVEKGIYQTSNFNVYAD
SFVIRGDEVRQIAPGQTGKIADYNYKLPDDFTGCV
IAWNSNNLDSKVGGNYNYLYRLFRKSNLKPFERDI
NGLTGTGVLTESNKKELPFQQFGRDIADTTDAVRD
PQTLEILDITPCSFGGVSVITPGTNTSNQVAVLYQ
DVNCTEVPVAIHADQLTPTWRVYSTGSNVFQTRAG
CLIGAEHVNNSYECDIPIGAGICASYQTQTNSPRR
ARSVASQSIIAYTMSLGAENSVAYSNNSIAIPTNE
TISVTTEILPVSMTKTSVDCTMYICGDSTECSNLL
LQYGSFCTQLNRALTGIAVEQDKNTQEVFAQVKQT
YKTPPIKDFGGFNFSQILPDPSKPSKRSFIEDLLE
NKVTLADAGFIKQYGDCLGDIAARDLICAQKFNGL
TVLPPLLTDEMIAQYTSALLAGTITSGWTFGAGAA
LQIPFAMQMAYRENGIGVTQNVLYENQKLIANQEN
SAIGKIQDSLSSTASALGKLQDVVNQNAQALNTLV
KQLSSNFGAISSVLNDILSRLDKVEAEVQIDRLIT
GRLQSLQTYVTQQLIRAAEIRASANLAATKMSECV
LGQSKRVDFCGKGYHLMSFPQSAPHGVVFLHVTYV
PAQEKNFTTAPAICHDGKAHFPREGVFVSNGTHWF
VTQRNFYEPQIITTDNTFVSGNCDVVIGIVNNTVY
DPLQPELDSFKEELDKYFKNHTSPDVDLGDISGIN
ASVVNIQKEIDRLNEVAKNLNESLIDLQELGKYEQ
YIKWPWYIWLGFIAGLIAIVMVTIMLCCMTSCCSC
LKGCCSCGSCCKFDEDDSEPVLKGVKLHYTAA

RBD SARS-COV-2 spike protein nucleic acid sequence
SEQ ID NO: 13 [symbol]
GACgccaccATGAGAGTCCAACCAACAGAATCTATTGTTAGATT
TCCTAATATTACAAACTTGTGCCCTTTTGGTGAAGTTTTTAACGCCACCA
GATTTGCATCTGTTTATGCTTGGAACAGGAAGAGAATCAGCAACTGTGTT
GCTGATTATTCTGTCCTATATAATTCCGCATCATTTTCCACTTTTAAGTG
TTATGGAGTGTCTCCTACTAAATTAAATGATCTCTGCTTTACTAATGTCT
ATGCAGATTCATTTGTAATTAGAGGTGATGAAGTCAGACAAATCGCTCCA
GGGCAAACTGGAAAGATTGCTGATTATAATTATAAATTACCAGATGATTT
TACAGGCTGCGTTATAGCTTGGAATTCTAACAATCTTGATTCTAAGGTTG
GTGGTAATTATAATTACCTGTATAGATTGTTTAGGAAGTCTAATCTCAAA
CCTTTTGAGAGAGATATTICAACTGAAATCTATCAGGCCGGTAGCACACC
TTGTAATGGTGTTGAAGGTTTTAATTGTTACTTTCCTTTACAATCATATG
GITTCCAACCCACTAATGGTGTTGGTTACCAACCATACAGAGTAGTAGTA
CTTTCTTTTGAACTTCTACATGCACCAGCAACTGTTTGTGGACCTAAAAA
Gtgataa

[symbol]

KOZAC sequence added (gcc acc, underlined) before the starting ATG (bold).
Secreted form tga taa added (double underlined) before NotI—this tga taa sequence is a “two stop codon” motif that interrupts protein synthesis, facilitating secretion into the extracellular medium (also included in other sequences, as described below).
Unique Restriction sites have been added respectively at 5′ end NheI and at the 3′ end, NotI (dash underlined)

RBD SARS-COV-2 spike protein nucleic acid
sequence-human codon optimized for
293F (HEK) cell expression.
SEQ ID NO: 14 [symbol]
GACgccaccATGAGAGTGCAGCCTACAGA
GTCTATCGTGCGGTT
CCCCAACATCACCAATCTGTGCCCTTTCGGCGAGG
TGTTCAACGCCACAAGATTTGCCAGCGTGTACGCC
TGGAACCGGAAGAGAATCAGCAACTGCGTGGCCGA
CTACAGCGTGCTGTACAATAGCGCCAGCTTCAGCA
CCTTCAAGTGCTACGGCGTGTCCCCTACCAAGCTG
AACGACCTGTGCTTCACCAATGTGTACGCCGACAG
CTTCGTGATCAGAGGCGACGAAGTTCGGCAGATCG
CTCCTGGACAGACAGGCAAGATCGCCGATTACAAC
TACAAGCTGCCCGACGACTTCACCGGCTGCGTGAT
CGCCTGGAATAGCAACAACCTGGACAGCAAAGTCG
GCGGCAACTACAACTACCTGTACCGGCTGTTCCGG
AAGTCCAACCTGAAGCCTTTCGAGCGGGACATCAG
CACCGAGATCTATCAGGCCGGCAGCACCCCTTGTA
ATGGCGTGGAAGGCTTCAACTGCTACTTCCCACTG
CAGTCCTACGGCTTCCAGCCTACAAACGGCGTGGG
CTACCAGCCTTATAGAGTGGTGGTGCTGAGCTTCG
AACTGCTGCATGCCCCTGCTACAGTGTGCGGCCCC
AAGAAGtgataa

[symbol]

KOZAC sequence added (gcc acc, underlined) before the starting ATG (bold).
Secreted form tga taa added (double underlined) before NotI
Unique Restriction sites have been added respectively at 5′ end NheI and at the 3′ end, NotI (dash underlined)

RBD SARS-COV-2 spike protein amino acid
sequence corresponding to SEQ ID NOS:
13 and 14
SEQ ID NO: 15
MRVQPTESIVRFPNITNLCPFGEVFNATRFASVYA
WNRKRISNCVADYSVLYNSASFSTFKCYGVSPTKL
NDLCFTNVYADSFVIRGDEVRQIAPGQTGKIADYN
YKLPDDFTGCVIAWNSNNLDSKVGGNYNYLYRLFR
KSNLKPFERDISTEIYQAGSTPCNGVEGFNCYFPL
QSYGFQPTNGVGYQPYRVVVLSFELLHAPATVCGP
KK

rigid EAAAK linker consensus amino acid sequence
SEQ ID NO: 16
A(EAAAK)ₙA (n = 2-5)
rigid (EAAAK)₃ linker nucleic acid sequence
SEQ ID NO: 17
GAA GCC GCC GCT AAA GAG GCC GCT GCC AAA GAA GCT GCT GCT AAG
rigid (EAAAK)₃ linker amino acid sequence
SEQ ID NO: 18
EAAAKEAAAKEAAAK
flexible GSₙ linker consensus amino acid sequence
SEQ ID NO: 19
(Gly-Gly-Gly-Gly-Ser)ₙ (n = 1-6)
flexible GS5 ((GGGGS)₁) linker amino acid sequence
SEQ ID NO: 20
GGGGS
flexible GS10 ((GGGGS)₂) linker amino acid sequence
SEQ ID NO: 21
GGGGSGGGGS
flexible GS15 ((GGGGS)₃) linker nucleic acid sequence
SEQ ID NO: 22
GGT GGT GGT GGT AGC GGT GGT GGC GGT TCA GGT GGC GGT GGT TCA
flexible GS15 ((GGGGS)₃) linker amino acid sequence
SEQ ID NO: 23
GGGGSGGGGSGGGGS
flexible GS20 ((GGGGS)₄) linker amino acid sequence
SEQ ID NO: 24
GGGGSGGGGSGGGGSGGGGS
flexible GS25 ((GGGGS)₅) linker amino acid sequence
SEQ ID NO: 25
GGGGSGGGGSGGGGSGGGGSGGGGS
HBSAg-(EAAAK)₃-RBD nucleic acid sequence
SEQ ID NO: 26

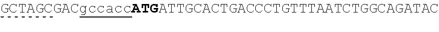

CCTGTTAGGTGGTCTGCCGACCGAACTGATTAGCAGTGCCGGTGGTCAGC
TGTTTTATAGCCGTCCGGTTGTTAGCGCAAATGGTGAACCGACCGTTAAA
CTGTATACCAGCGTTGAAAATGCACAGCAGGATAAAGGTATTGCAATTCC
GCATGATATTGATCTGGGTGAAAGCCGTGTTGTGATTCAGGATTATGATA
ATCAGCATGAACAGGATCGTCCGACACCGAGTCCGGCACCGAGCCGTCCG
TTTAGCGTTCTGCGTGCAAATGATGTTCTGTGGCTGAGCCTGACCGCAGC
AGAATATGATCAGAGCACCTATGGTAGCAGCACCGGTCCGGTTTATGTTA
GCGATAGCGTTACCCTGGTTAATGTTGCAACCGGTGCACAGGCAGTTGCA
CGTAGCCTGGATTGGACCAAAGTGACCCTGGATGGTCGTCCGCTGAGCAC
CATTCAGCAGTATAGCAAAACCTTTTTTGTTCTGCCGCTGCGTGGTAAAC
TGAGCTTTTGGGAAGCAGGCACCACCAAAGCAGGTTATCCGTATAACTAT
TGTTGCAATTAGCACCTATACCACCAGTTTAGGTGCAGGTCCGGTTAGCA

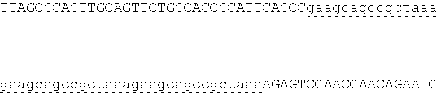

TATTGTTAGATTTCCTAATATTACAAACTTGTGCCCTTTTGGTGAAGTTT
TTAACGCCACCAGATTTGCATCTGTTTATGCTTGGAACAGGAAGAGAATC
AGCAACTGTGTTGCTGATTATTCTGTCCTATATAATTCCGCATCATTTTC
CACTTTTAAGTGTTATGGAGTGTCTCCTACTAAATTAAATGATCTCTGCT
TTACTAATGTCTATGCAGATTCATTTGTAATTAGAGGTGATGAAGTCAGA
CAAATCGCTCCAGGGCAAACTGGAAAGATTGCTGATTATAATTATAAATT
ACCAGATGATTTTACAGGCTGCGTTATAGCTTGGAATTCTAACAATCTTG
ATTCTAAGGTTGGTGGTAATTATAATTACCTGTATAGATTGTTTAGGAAG
TCTAATCTCAAACCTTTTGAGAGAGATATTTCAACTGAAATCTATCAGGC
CGGTAGCACACCTTGTAATGGTGTTGAAGGTTTTAATTGTTACTTTCCTT
TACAATCATATGGTTTCCAACCCACTAATGGTGTTGGTTACCAACCATAC
AGAGTAGTAGTACTTTCTTTTGAACTTCTACATGCACCAGCAACTGTTTG

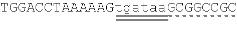

KOZAC sequence added (gcc acc, underlined) before the starting ATG (bold).
Secreted form tga taa added (double underlined) before NotI
Unique Restriction sites have been added respectively at 5′ end NheI and at the 3′ end, NotI (dash underlined)
The bold and dotted underlined sequence corresponds to the (EAAAK)₃ linker.

HBSAg-(EAAAK)₃-RBD nucleic acid sequence human codon optimised for 293f
(HEK) cell expression
SEQ ID NO: 27

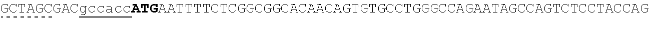

CAATCACAGCCCCACCAGCTGTCCTCCAACCTGTCCTGGCTACAGATGGATGTGCCTGCGGCGGTTCATCATCT
TTCTGTTCATCCTGCTGCTGTGCCTGATCTTCCTGCTGGTGCTGCTGGATTACCAGGGAATGCTGCCTGTGTGT
CCTCTGATCCCTGGCAGCAGCACAACAAGCACAGGCCCTTGCAGAACCTGCACAACACCAGCTCAGGGCACCA
GCATGTACCCTAGCTGCTGTTGTACCAAGCCTAGCGACGGCAACTGCACATGCATCCCCATTCCTAGCAGCTG
GGCCTTCGGCAAGTTTCTGTGGGAATGGGCCAGCGCCAGATTTTCCGAAGCCGCCGCTAAAGAGGCCGCTGC

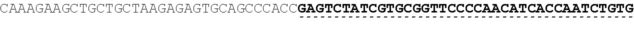

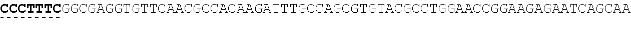

CTGCGTGGCCGACTACAGCGTGCTGTACAATAGCGCCAGCTTCAGCACCTTCAAGTGCTACGGCGTGTCCCCT
ACCAAGCTGAACGACCTGTGCTTCACCAATGTGTACGCCGACAGCTTCGTGATCAGAGGCGACGAAGTTCGG
CAGATCGCTCCTGGACAGACAGGCAAGATCGCCGATTACAACTACAAGCTGCCCGACGACTTCACCGGCTGC
GTGATCGCCTGGAATAGCAACAACCTGGACAGCAAAGTCGGCGGCAACTACAACTACCTGTACCGGCTGTTCC
GGAAGTCCAACCTGAAGCCTTTCGAGCGGGACATCAGCACCGAAATCTACCAGGCCGGCAGCACCCCTTGTA
ATGGCGTGGAAGGCTTCAACTGCTACTTCCCACTGCAGTCCTACGGCTTCCAGCCTACAAACGGCGTGGGCTA
CCAGCCTTATAGAGTGGTGGTGCTGAGCTTCGAACTGCTGCATGCCCCTGCTACAGTGTGCGGCCCCAAGAAG

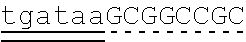

KOZAC sequence added (gcc acc, underlined) before the starting ATG (bold).
Secreted form tga taa added (double underlined) before NotI
Unique Restriction sites have been added respectively at 5′ end NheI and at the 3′ end, NotI (dash underlined)
The bold and dotted underlined sequence corresponds to the (EAAAK)₃ linker.

HBSAg-(EAAAK)₃-RBD amino acid sequence corresponding to SEQ ID NOS: 26 AND 27
SEQ ID NO: 28
MNFLGGTTVCLGQNSQSPTSNHSPTSCPPTCPGYRWMCLRRFIIFLFILLLCLIFLLVLLDYQGMLPVCPLIPGSSTTS

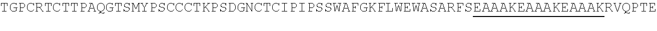

SIVRFPNITNLCPFGEVFNATRFASVYAWNRKRISNCVADYSVLYNSASFSTFKCYGVSPTKLNDLCFTNVYADSFVIR
GDEVRQIAPGQTGKIADYNYKLPDDFTGCVIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPKK

The (EAAAK)₃ linker is underlined.

HEV-GS15-RBD nucleic acid sequence
SEQ ID NO: 29

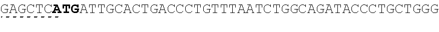

TGGTCTGCCGACCGAACTGATTAGCAGTGCCGGTGGTCAGCTGTTTTATA
GCCGTCCGGTTGTTAGCGCAAATGGTGAACCGACCGTTAAACTGTATACC
AGCGTTGAAAATGCACAGCAGGATAAAGGTATTGCAATTCCGCATGATAT
TGATCTGGGTGAAAGCCGTGTTGTGATTCAGGATTATGATAATCAGCATG
AACAGGATCGTCCGACCCCGAGTCCGGCACCGAGCCGTCCGTTTAGCGTT
CTGCGTGCAAATGATGTTCTGTGGCTGAGCCTGACCGCAGCAGAATATGA
TCAGAGCACCTATGGTAGCAGCACCGGTCCGGTTTATGTTAGCGATAGCG
TTACCCTGGTTAATGTTGCAACCGGTGCACAGGCAGTTGCACGTAGCCTG
GATTGGACCAAAGTGACCCTGGATGGTCGTCCGCTGAGCACCATTCAGCA
GTATAGCAAAACCTTTTTTGTTCTGCCGCTGCGTGGTAAACTGAGCTTTT
GGGAAGCAGGCACCACCAAAGCAGGTTATCCGTATAACTATAATACCACC
GCAAGCGATCAGCTGCTGGTTGAAAACGCAGCAGGTCATCGTGTTGCAAT
TAGCACCATACCACCAGTCTGGGTGCAGGTCCGGTTAGCATTAGCGCAG

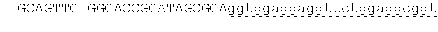

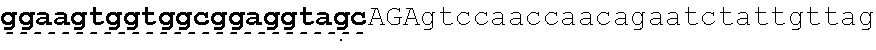

atttcctaatattacaaacttgtgcccttttggtgaagtttttaacgcca
ccagatttgcatctgtttatgcttggaacaggaagagaatcagcaactgt
gttgctgattattctgtcctatataattccgcatcattttccacttttaa
gtgttatggagtgtctcctactaaattaaatgatctctgctttactaatg
tctatgcagattcatttgtaattagaggtgatgaagtcagacaaatcgct
ccagggcaaactggaaagattgctgattataattataaattaccagatga
ttttacaggctgcgttatagcttggaattctaacaatcttgattctaagg
ttggtggtaattataattacctgtatagattgtttaggaagtctaatctc
aaaccttttgagagagatatttcaactgaaatctatcaggccggtagcac
accttgtaatggtgttgaaggttttaattgttactttcctttacaatcat
atggtttccaacccactaatggtgttggttaccaaccatacagagtagta
gtactttettttgaacttctacatgcaccagcaactgtttgtggacctaa

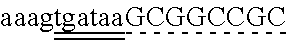

starting ATG (bold)
Unique Restriction sites have been added respectively at 5′ end, SacI and at the 3′ end, NotI (dash underlined)
Secreted form tga taa added (double underlined) before NotI
The bold and dotted underlined sequence corresponds to the GS15 linker.

HEV-GS15-RBD nucleic acid sequence optimized for E. coli expression
SEQ ID NO: 30

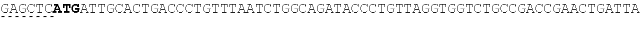

GCAGTGCCGGTGGTCAGCTGTTTTATAGCCGTCCGGTTGTTAGCGCAAATGGTGAACCGACCGTTAAACTGTA
TACCAGCGTTGAAAATGCACAGCAGGATAAAGGTATTGCAATTCCGCATGATATTGATCTGGGTGAAAGCCGT
GTTGTGATTCAGGATTATGATAATCAGCATGAACAGGATCGTCCGACACCGAGTCCGGCACCGAGCCGTCCGT
TTAGCGTTCTGCGTGCAAATGATGTTCTGTGGCTGAGCCTGACCGCAGCAGAATATGATCAGAGCACCTATGG
TAGCAGCACCGGTCCGGTTTATGTTAGCGATAGCGTTACCCTGGTTAATGTTGCAACCGGTGCACAGGCAGTT
GCACGTAGCCTGGATTGGACCAAAGTGACCCTGGATGGTCGTCCGCTGAGCACCATTCAGCAGTATAGCAAA
ACCTTTTTTGTTCTGCCGCTGCGTGGTAAACTGAGCTTTTGGGAAGCAGGCACCACCAAAGCAGGTTATCCGT
ATAACTATAATACCACCGCAAGCGATCAGCTGCTGGTTGAAAACGCAGCAGGTCATCGTGTTGCAATTAGCAC

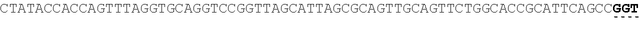

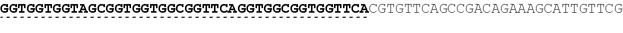

TTTTCCGAATATCACCAATCTGTGTCCGTTTGGCGAAGTTTTTAATGCAACCCGTTTTGCAAGCGTTTATGCCTG
GAATCGTAAACGTATTAGCAATTGCGTTGCCGATTATAGCGTGCTGTATAATAGCGCAAGCTTTAGCACCTTTA
AATGCTATGGTGTTAGCCCGACCAAACTGAATGATCTGTGTTTTACCAATGTGTATGCCGATAGCTTTGTGATT
CGTGGTGATGAAGTTCGTCAGATTGCACCGGGTCAGACCGGTAAAATTGCAGATTATAACTACAAACTGCCG
GATGATTTTACGGGTTGTGTTATTGCATGGAATAGCAATAACCTGGATAGCAAAGTTGGTGGCAACTATAACT
ATCTGTATCGCCTGTTTCGTAAGAGCAATCTGAAACCGTTTGAACGTGATATTAGCACCGAAATTTATCAGGCA
GGTAGCACCCCGTGCAATGGTGTTGAAGGTTTTAATTGTTATTTTCCGCTGCAGAGCTATGGTTTTCAGCCTAC
CAATGGTGTGGGTTATCAGCCGTATCGTGTTGTTGTTCTGTCATTTGAACTGCTGCATGCACCGGCAACCGTTT

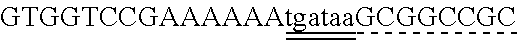

Secreted form tga taa added (double underlined) before NotI
Unique Restriction sites have been added respectively at 5′ end, SacI and at the 3′ end, NotI (dash underlined)
The bold and dotted underlined sequence corresponds to the GS15 linker.

HEV-GS15-RBD amino acid sequence corresponding to SEQ ID NO: 29 and 30
SEQ ID NO: 31
MIALTLFNLADTLLGGLPTELISSAGGQLFYSRPVVSANGEPTVKLYTSVENAQQDKGIAIPHDIDLGESRVVIQDYD
NQHEQDRPTPSPAPSRPFSVLRANDVLWLSLTAAEYDQSTYGSSTGPVYVSDSVTLVNVATGAQAVARSLDWTKV
TLDGRPLSTIQQYSKTFFVLPLRGKLSFWEAGTTKAGYPYNYNTTASDQLLVENAAGHRVAISTYTTSLGAGPVSISA

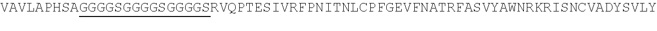

NSASFSTFKCYGVSPTKLNDLCFTNVYADSFVIRGDEVRQIAPGQTGKIADYNYKLPDDFTGCVIAWNSNNLDSKV
GGNYNYLYRLFRKSNLKPFERDISTEIYQAGSTPCNGVEGFNCYFPLQSYGFQPTNGVGYQPYRVVVLSFELLHAPA
TVCGPKK

The GS15 linker is underlined

HBSAg-(EAAAK)₃-full-length SARS-Cov-2 spike protein nucleic acid
sequence human codon optimised for 293f (HEK) cell expression
SEQ ID NO: 32

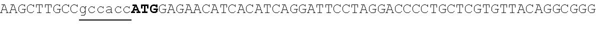

GTTTTTCTTGTTGACAAGAATCCTCACAATACCACAGAGTCTAGACTCGTGGTGGACTTCTCTCAATT
TTCTAGGGGGATCACCCGTGTGTCTGGGCCAAAATTCGCAGTCCCCAACCTCCAATCACTCACCAACC
TCTTGTCCTCCAATTTGTCCTGGCTATCGCTGGATGTGTCTGCGGCGTTTTATCATATTCCTCTTCAT
CCTGCTGCTATGCCTCATCTTCTTGTTGGTTCTTCTGGACTACCAGGGTATGTTGCCCGTTTGTCCTC
TAATTCCAGGATCAACAACTACCAACACGGGACCATGCAAGACCTGCACGACTCCTGCTCAAGGAAAC
TCTATGTTTCCCTCTTGTTGCTGTACAAAACCTACCGACGGAAACTGCACTTGTATTCCCATCCCATC
ATCCTGGGCTTTCGCAAAATACCTATGGGAGTGGGCCTCAGTCCGTTTCTCCTGGCTCAGTTTACTAG
TGCCATTTGTTCAGTGGTTCGTAGGGCTTTCCCCCACTGTTTGGCTTTCCGCTATATGGATGATGTGG
TATTGGGGGCCAAGTCTGTACAGCATCGTGAGTCCCTTTATACCTCTATTACCAATTTTCTTTTGTCT

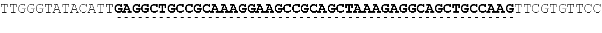

TGGTTCTGCTGCCCCTGGTGTCTAGCCAGTGCGTGAACCTGACCACCAGAACACAGCTGCCTCCAGCC
TACCCAGGACCTGTTCCTGCCTTTCTTCTCCAACGTGACCTGGTTCCACGCCATCCATGTGTCTGGCA
CCAACGGCACCAAGAGATTCGACAACCCCGTGCTGCCTTTCAACGACGGGGTGTACTTTGCCTCCACC
GAGAAGTCCAACATCATCAGAGGCTGGATCTTCGGCACAACCCTGGACAGCAAGACCCAGAGCCTGCT
GATCGTGAACAACGCCACCAACGTGGTCATCAAAGTGTGCGAGTTCCAGTTCTGCAACGACCCCTTCC
TGGGCGTCTACTACCACAAGAACAACAAGTCCTGGATGGAATCCGAGTTCCGGGTGTACTCCTCCGCC
AACAACTGCACCTTCGAATACGTGTCCCAGCCTTTCCTGATGGACCTGGAAGGCAAGCAGGGCAACTT
CAAGAACCTGCGCGAGTTCGTGTTCAAGAACATCGACGGCTACTTCAAGATCTACTCCAAGCACACCC
CTATCAACCTCGTGCGGGATCTGCCTCAGGGCTTCTCTGCTCTGGAACCCCTGGTGGATCTGCCCATC
GGCATCAACATCACCCGGTTTCAGACCCTGCTGGCCCTGCACCGGTCTTATTTGACCCCTGGCGACTC
CTCTTCTGGCTGGACTGCTGGCGCCGCTGCTTACTATGTGGGCTACCTGCAGCCTCGGACCTTTCTGC
TGAAGTACAACGAGAATGGCACCATCACCGACGCCGTGGACTGTGCTCTGGATCCTCTGTCCGAGACA
AAGTGCACCCTGAAGTCCTTCACCGTGGAAAAGGGCATCTACCAGACCTCCAACTTCCGGGTGCAGCC
CACCGAGTCTATCGTGCGGTTCCCTAACATCACCAACCTGTGTCCTTTCGGCGAGGTGTTCAATGCCA
CCAGATTCGCCTCTGTGTACGCCTGGAACCGGAAGCGGATCTCTAACTGCGTGGCCGACTACAGCGTG
CTGTACAACTCCGCCTCCTTCAGCACCTTCAAGTGCTACGGCGTGTCCCCTACAAAGCTGAACGACCT
GTGCTTCACAAACGTGTACGCCGACAGCTTCGTGATCCGGGGAGATGAAGTGCGGCAGATCGCTCCTG
GACAGACCGGCAAGATCGCCGATTACAACTACAAGCTGCCCGACGACTTCACCGGCTGTGTGATCGCT
TGGAACTCCAACAACCTGGACTCCAAAGTCGGCGGCAACTACAACTACCTGTACCGGCTGTTCCGGAA
GTCTAACCTGAAGCCTTTCGAGCGGGACATCAGCACCGAGATCTACCAGGCTGGCAGCACCCCTTGTA
ACGGCGTGGAAGGCTTCAACTGCTACTTCCCACTGCAGTCCTACGGCTTTCAGCCTACCAATGGCGTG
GGCTATCAGCCCTACAGAGTGGTGGTGCTGTCCTTCGAGCTGCTGCATGCTCCTGCTACCGTGTGCGG
CCCTAAGAAATCTACCAACCTGGTCAAGAACAAATGCGTGAACTTCAACTTCAACGGCCTGACCGGCA
CCGGCGTGCTGACAGAGTCCAACAAGAAGTTCCTGCCATTCCAGCAGTTCGGCCGGGATATCGCCGAT
ACCACAGATGCCGTCAGGGACCCTCAGACACTGGAAATCCTGGACATCACCCCTTGCTCCTTCGGCGG
AGTGTCTGTGATCACCCCAGGCACCAACACCTCTAACCAGGTGGCCGTGCTGTATCAGGACGTGAACT
GTACCGAGGTGCCCGTGGCTATCCATGCCGATCAGCTGACCCCTACATGGCGCGTGTACTCCACCGGC
TCTAACGTGTTCCAGACAAGAGCTGGCTGTCTGATCGGCGCTGAGCACGTGAACAATTCCTACGAGTG
CGACATCCCCATCGGAGCCGGAATCTGCGCCTCTTATCAGACCCAGACCAACTCTCCCAGACGGGCCA
GATCTGTGGCCAGCCAGTCTATCATTGCTTACACCATGAGCCTGGGCGCCGAGAACTCTGTGGCCTAC
AGCAACAACTCTATCGCTATCCCCACCAACTTCACCATCTCCGTGACCACAGAGATCCTGCCAGTGTC
CATGACCAAGACCAGCGTGGACTGCACCATGTACATCTGCGGCGACTCTACCGAGTGCTCCAACCTGC
TGCTCCAGTACGGCTCCTTCTGCACCCAGCTGAATAGAGCCCTGACCGGAATCGCCGTGGAACAGGAC
AAGAACACCCAAGAGGTGTTCGCCCAAGTGAAGCAGATCTACAAGACCCCTCCTATCAAGGACTTCGG
CGGCTTCAATTTCTCCCAGATTCTGCCCGATCCTAGCAAGCCCTCCAAGCGGTCTTTCATCGAGGACC
TGCTGTTCAACAAAGTGACACTGGCCGACGCCGGCTTCATCAAGCAGTACGGCGACTGTCTGGGCGAC
ATTGCCGCTAGGGATCTGATCTGCGCCCAGAAGTTTAACGGACTGACAGTGCTGCCTCCTCTGCTGAC
CGATGAGATGATCGCCCAGTACACCTCCGCACTGCTGGCTGGCACAATCACCTCTGGATGGACATTTG
GCGCTGGCGCTGCTCTGCAAATCCCATTCGCTATGCAAATGGCCTACCGGTTCAACGGCATCGGCGTG
ACCCAGAATGTGCTGTACGAGAACCAGAAGCTGATCGCCAACCAGTTCAACAGCGCCATCGGAAAGAT
CCAGGACAGCCTGTCCAGCACCGCTTCTGCCCTGGGAAAGCTGCAGGATGTGGTCAACCAGAACGCTC
AGGCCCTGAACACCCTCGTGAAGCAGCTGTCTAGCAACTTCGGCGCCATCTCCTCTGTGCTGAACGAT
ATCCTGAGCCGGCTGGACAAGGTGGAAGCCGAGGTGCAGATCGACAGACTGATCACCGGACGGCTGCA
GTCCCTGCAGACCTATGTTACCCAGCAGCTGATCCGGGCTGCCGAGATTAGAGCCTCTGCCAATCTGG
CCGCAACCAAGATGTCTGAGTGTGTGCTGGGACAGTCCAAGAGAGTGGACTTCTGCGGCAAGGGCTAC
CACCTGATGAGCTTCCCTCAGTCTGCTCCTCACGGCGTGGTGTTTCTGCACGTGACCTACGTGCCCGC
TCAAGAGAAGAACTTTACCACCGCTCCTGCCATCTGCCACGACGGCAAGGCTCACTTTCCTAGAGAAG
GCGTGTTCGTGTCTAACGGCACCCATTGGTTCGTGACACAGCGGAACTTCTACGAGCCCCAGATCATC
ACCACCGACAACACCTTCGTGTCCGGCAACTGCGACGTCGTGATCGGAATTGTGAACAATACCGTGTA
CGACCCTCTGCAGCCCGAGCTGGACTCCTTCAAAGAGGAACTGGACAAGTACTTTAAGAACCACACAA
GCCCCGACGTGGACCTGGGAGACATCTCTGGCATCAACGCCTCCGTGGTCAACATCCAGAAAGAGATC
GACCGGCTGAACGAGGTGGCCAAGAATCTGAACGAGTCCCTGATCGACCTGCAAGAACTGGGGAAGTA
CGAGCAGTACATCAAGTGGCCCTGGTACATCTGGCTGGGCTTTATCGCTGGCCTGATCGCTATCGTGA
TGGTCACAATCATGCTGTGCTGTATGACCTCCTGTTGCTCCTGCCTGAAGGGCTGCTGCTCTTGCGGC
TCTTGCTGCAAGTTCGACGAGGACGACTCTGAGCCCGTGCTGAAAGGCGTGAAGCTGCACTATACCTG
ATGACTCGAG

KOZAC sequence added (gcc acc, underlined) before the starting ATG (bold).
The bold and dotted underlined sequence corresponds to the (EAAAK)₃ linker.

HBSAg-(EAAAK)₃-full-length 2019-nCoV spike protein
amino acid sequence corresponding to SEQ ID NO: 32
SEQ ID NO: 33
MENITSGFLGPLLVLQAGFFLLTRILTIPQSLDSWWTSLNFLGGSPVCLG
QNSQSPTSNHSPTSCPPICPGYRWMCLRRFIIFLFILLLCLIFLLVLLDY
QGMLPVCPLIPGSTTTNTGPCKTCTTPAQGNSMFPSCCCTKPTDGNCTCI
PIPSSWAFAKYLWEWASVRFSWLSLLVPFVQWFVGLSPTVWLSAIWMMWY
WGPSLYSIVSPFIPLLPIFFCLWVYIEAAAKEAAAKEAAAKFVFLVLLPL
VSSQCVNLTTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTL
DSKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTPI
NLVRDLPQGFSALEPLVDLPIGINITRFQTLLALHRSYLTPGDSSSGWTA
GAAAYYVGYLQPRTFLLKYNENGTITDAVDCALDPLSETKCTLKSFTVEK
GIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQI
APGQTGKIADYNYKLPDDFTGCVIAWNSNNLDSKVGGNYNYLYRLFRKSN
LKPFERDISTEIYQAGSTPCNGVEGFNCYFPLQSYGFQPTNGVGYQPYRV
VVLSFELLHAPATVCGPKKSTNLVKNKCVNFNFNGLTGTGVLTESNKKFL
PFQQFGRDIADTTDAVRDPQTLEILDITPCSFGGVSVITPGTNTSNQVAV
LYQDVNCTEVPVAIHADQLTPTWRVYSTGSNVFQTRAGCLIGAEHVNNSY
ECDIPIGAGICASYQTQTNSPRRARSVASQSIIAYTMSLGAENSVAYSNN
SIAIPTNFTISVTTEILPVSMTKTSVDCTMYICGDSTECSNLLLQYGSFC
TQLNRALTGIAVEQDKNTQEVFAQVKQIYKTPPIKDFGGFNFSQILPDPS
KPSKRSFIEDLLFNKVTLADAGFIKQYGDCLGDIAARDLICAQKFNGLTV
LPPLLTDEMIAQYTSALLAGTITSGWTFGAGAALQIPFAMQMAYRFNGIG
VTQNVLYENQKLIANQFNSAIGKIQDSLSSTASALGKLQDVVNQNAQALN
TLVKQLSSNFGAISSVLNDILSRLDKVEAEVQIDRLITGRLQSLQTYVTQ
QLIRAAEIRASANLAATKMSECVLGQSKRVDFCGKGYHLMSFPQSAPHGV
VFLHVTYVPAQEKNFTTAPAICHDGKAHFPREGVFVSNGTHWFVTQRNFY
EPQIITTDNTFVSGNCDVVIGIVNNTVYDPLQPELDSFKEELDKYFKNHT
SPDVDLGDISGINASVVNIQKEIDRLNEVAKNLNESLIDLQELGKYEQYI
KWPWYIWLGFIAGLIAIVMVTIMLCCMTSCCSCLKGCCSCGSCCKFDEDD
SEPVLKGVKLHYT

The (EAAAK)₃ linker is underlined

1. A combined influenza-COVID-19 vaccine comprising:
(a) an influenza haemagglutinin (HA) protein or an immunogenic fragment thereof; and
(b) one or more protein antigens derived from SARS-CoV-2 or an immunogenic fragment thereof;
wherein the influenza haemagglutinin (HA) protein or an immunogenic fragment thereof and the one or more protein antigens derived from SARS-CoV-2 or an immunogenic fragment thereof are capable of eliciting an immune response and protection against both influenza and COVID-19.

2. The combined influenza-COVID-19 vaccine of claim 1, which further comprises an influenza neuraminidase (NA) protein or an immunogenic fragment thereof.

3. The combined influenza-COVID-19 vaccine of claim 1, wherein:
(a) the influenza HA protein or immunogenic fragment thereof is:
(i) comprised in an inactivated influenza virion;
(ii) a recombinant HA or immunogenic fragment thereof;
(iii) a fusion protein comprising HA protein or an immunogenic fragment thereof; or
(iv) encoded by an RNA or DNA vaccine; and/or
(b) the influenza NA protein or immunogenic fragment thereof is:
(i) comprised in an inactivated influenza virion;
(ii) a recombinant NA protein or immunogenic fragment thereof;
(iii) a fusion protein comprising NA or an immunogenic fragment thereof; or
(iv) encoded by an RNA or DNA vaccine; and/or
(c) the one or more protein antigens derived from SARS-CoV-2 or an immunogenic fragment thereof is:
(i) at least one recombinant SARS-CoV-2 spike protein or immunogenic fragment thereof;
(ii) at least one fusion protein comprising a SARS-CoV-2 spike protein or immunogenic fragment thereof;
(iii) at least one virus-like particle (VLP) comprising a SARS-CoV-2 spike protein or immunogenic fragment thereof;
(iv) encoded by at least one polynucleotide encoding a recombinant SARS-CoV-2 spike protein or immunogenic fragment thereof; or
(v) encoded by at least one RNA or DNA vaccine.

4. The combined influenza-COVID-19 vaccine of claim 1,
wherein the influenza HA protein or immunogenic fragment thereof and the influenza NA protein or immunogenic fragment thereof are comprised in an inactivated influenza virion and the one or more protein antigens derived from SARS-CoV-2 or an immunogenic fragment thereof is:
(i) at least one fusion protein comprising a SARS-CoV-2 spike protein or immunogenic fragment thereof or
(ii) at least one virus-like particle (VLP) comprising a SARS-CoV-2 spike protein or immunogenic fragment thereof.

5. The combined influenza-COVID-19 vaccine of claim 1, wherein:
(a) the influenza HA or immunogenic fragment thereof is comprised in a live attenuated influenza virion;
(b) the influenza NA or immunogenic fragment thereof is comprised in a live attenuated influenza virion; and/or
(c) the one or more protein antigens derived from SARS-CoV-2 or an immunogenic fragment thereof is comprised in a live viral vector.

6. The combined influenza-COVID-19 vaccine of claim 5, wherein the live viral vector comprising the one or more protein antigens derived from SARS-CoV-2 or an immunogenic fragment thereof is:
(a) an adenoviral vector;
(b) a measles virus vector;
(c) a mumps virus vector;
(d) a rubella virus vector;
(e) a varicella virus vector;
(f) a polio virus vector; or
(g) a yellow fever virus vector.

7. The combined influenza-COVID-19 vaccine of claim 1, further comprising an adjuvant.

8. The combined influenza-COVID-19 vaccine of claim 7, wherein said adjuvant is a stimulator of a cellular (Th1) response and a humoral (Th2) immune response.

9. The combined influenza-COVID-19 vaccine of claim 7, wherein said adjuvant comprises a squalene oil-in-water emulsion, an aluminium salt or a monophosphoryl Lipid A (MPL).

10. The combined influenza-COVID-19 vaccine of claim 1, wherein the one or more protein antigens derived from SARS-CoV-2 is selected from:
(a) a spike protein from SARS-CoV-2 having at least 90% identity with SEQ ID NO: 1, or a fragment thereof that has a common antigenic cross-reactivity with said spike protein;
(b) a fusion protein comprising a spike protein from SARS-CoV-2 having at least 90% identity with SEQ ID NO: 1, or a fragment thereof that has a common antigenic cross-reactivity with said spike protein;
(c) a VLP comprising a spike protein from SARS-CoV-2 having at least 90% identity with SEQ ID NO: 1, or a fragment thereof that has a common antigenic cross-reactivity with said spike protein;
(d) a polynucleotide encoding a spike protein from SARS-CoV-2 having at least 90% identity with SEQ ID NO: 1, or a fragment thereof that has a common antigenic cross-reactivity with said spike protein; or
(e) a viral vector, RNA vaccine or DNA plasmid that expresses a spike protein from SARS-CoV-2 having at least 90% identity with SEQ ID NO: 1, or a fragment thereof, that has a common antigenic cross-reactivity with said spike protein
wherein optionally the fragment of the SARS-CoV-2 spike protein comprises or consists of the receptor-binding domain (RBD) of the SARS-CoV-2 spike protein, optionally having at least 90% identity with SEQ ID NO: 15.

11. The combined influenza-COVID-19 vaccine claim 1, wherein the one or more protein antigens derived from SARS-CoV-2 is a fusion protein comprising a SARS-CoV-2 spike protein or immunogenic fragment thereof and further comprising:
(a) the Hepatitis B surface antigen, or a fragment thereof that has a common antigenic cross-reactivity with said Hepatitis B surface antigen;
(b) the HPV 18 L1 protein, or a fragment thereof that has a common antigenic cross-reactivity with said HPV 18 L1 protein;
(c) the Hepatitis E P239 protein, or a fragment thereof that has a common antigenic cross-reactivity with said Hepatitis E P239 protein; and/or
(d) the HPV 16 L1 protein, or a fragment thereof that has a common antigenic cross-reactivity with said HPV 16 L1 protein.

12. The combined influenza-COVID-19 vaccine of claim 11, wherein:
the fusion protein is encoded by a polynucleotide which comprises or consists of a nucleic acid sequence having at least 90% identity with any one of SEQ ID NO: 3, 5, 6, 8, 26, 27, 29, 30 or 32; and/or
the fusion protein comprises or consists of an amino acid sequence having at least 90% identity with any one of SEQ ID NO: 9, 10, 11, 12, 28, 31 or 33.

13. The combined influenza-COVID-19 vaccine of claim 1,
wherein the one or more protein antigens derived from SARS-CoV-2 is a VLP comprising a SARS-CoV-2 spike protein or immunogenic fragment thereof,
wherein said VLP comprises or consists of a fusion protein as defined in claim 11 or 12.

14. The combined influenza-COVID-19 vaccine of claim 1,
wherein the influenza HA or immunogenic fragment thereof and the influenza NA or immunogenic fragment thereof are comprised in:
a seasonal influenza vaccine,
and optionally the seasonal influenza vaccine is a seasonal 3-valent influenza vaccine or a seasonal 4-valent influenza vaccine;
a monovalent pandemic influenza vaccine; or
a universal influenza vaccine.

15-16. (canceled)

17. A method of immunising a subject against both influenza and COVID-19 comprising:
(a) providing a combined influenza-COVID-19 vaccine as set forth in claim 1; and
(b) administering to said subject a therapeutically effective amount of the combined influenza-COVID-19 vaccine.

18. The method of claim 17, wherein the combined influenza-COVID-19 vaccine is administered at intervals of 10 to 14 months, or the combined influenza-COVID-19 vaccine is administered at intervals of about 12 months.


SEQUENCE LISTING (33 sequences)

SEQ ID NO: 1 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - DNA, 3839 bp, Artificial Sequence
  SARS-CoV2 spike protein nucleic acid - optimised for E. coli expression
gagctcatgtttgtttttctggttctgctgccgctggttagcagccagtgtgttaatctgaccacacgtacccagctgcctccggcatataccaatagctttacccgtggtgtttattatccggacaaagtttttcgtagcagcgttctgcatagcacccaggacctgtttctgccgttttttagcaatgttacctggtttcatgccattcatgttagcggcaccaatggcaccaaacgttttgataatccggtgctgccgtttaatgatggtgtgtattttgcaagcaccgaaaaaagcaacattattcgcggttggatttttggtacaaccctggatagcaaaacccagagcctgctgattgttaataatgccaccaatgtggtgatcaaagtgtgcgaatttcagttttgcaatgatccgtttctgggcgtgtattaccacaaaaataacaagagctggatggaaagcgaatttcgtgtttatagcagcgccaataattgcacctttgaatatgttagccagccgtttctgatggatctggaaggtaaacagggtaactttaaaaacctgcgcgagttcgtgttcaaaaacatcgatggttacttcaaaatctatagcaaacacaccccgattaatctggttcgtgatctgccgcagggttttagcgcactggaaccgctggttgatctgccaattggtattaacattacccgttttcagaccctgctggcactgcatcgtagctatctgacaccgggtgatagcagcagcggttggaccgcaggcgcagcagcatattatgttggttatctgcagcctcgtacctttctgctgaaatataacgaaaatggcacaattaccgatgccgttgattgtgccctggatccgctgagcgaaaccaaatgtaccctgaaaagctttaccgttgagaaaggtatttatcagaccagcaattttcgtgtgcagccgaccgaaagcattgttcgttttccgaatatcaccaatctgtgtccgtttggcgaagtttttaatgcaacccgttttgccagcgtttatgcatggaatcgtaaacgtattagcaattgcgttgccgattatagcgttctgtataatagcgcaagcttcagcacctttaaatgctatggtgttagcccgaccaaactgaatgatctgtgttttaccaatgtgtatgccgatagctttgtgattcgtggtgatgaagttcgtcagattgcaccgggtcagaccggtaaaattgcagattataactataaactgccggatgattttacgggttgtgttattgcctggaatagcaataatctggacagcaaagttggtggcaactataactatctgtatcgcctgtttcgtaagagcaatctgaaaccgtttgaacgtgatattagcaccgagatttatcaggcaggtagcaccccgtgtaatggtgttgaaggttttaattgctattttccgctgcagagctatggttttcagccgacaaatggtgtgggttatcagccgtatcgtgttgttgttctgtcatttgaactgctgcatgcaccggcaaccgtttgtggtccgaaaaaaagtaccaatctggtgaaaaataagtgcgtgaactttaactttaatggtctgaccggcaccggtgttctgaccgaaagtaacaaaaaattcctgccgtttcagcagtttggccgtgatattgcagataccaccgatgcagttcgcgatccgcagacactggaaattctggatattaccccgtgcagctttggtggtgtttcagttattacaccgggtacaaataccagcaatcaggttgcagttctgtatcaggatgttaattgtaccgaagttccggttgcaattcatgcagatcagctgaccccgacctggcgtgtgtatagcaccggtagcaatgtgtttcagacacgtgcaggttgtctgattggtgcagaacatgtgaataatagctatgaatgcgatattccgattggtgcgggtatttgtgccagctatcagacccagaccaatagtccgcgtcgtgcacgtagcgttgcaagccagagcattattgcctataccatgagcctgggtgcagaaaatagcgttgcctatagtaataacagcattgccattccgaccaactttaccattagcgttaccaccgaaattctgccggttagcatgaccaaaaccagcgttgattgcaccatgtatatttgtggtgatagtaccgaatgtagcaatctgctgctgcagtatggtagcttttgcacccagctgaatcgtgcactgaccggtattgcagttgaacaggataaaaacacgcaagaagtttttgcacaggtcaagcagatctataaaacccctccgattaaagattttggcggtttcaattttagccagatcctgccggatccgagcaaaccgagtaaacgtagctttattgaagatctgctgttcaacaaagtgaccctggcagatgcaggttttatcaaacagtatggtgattgcctgggcgatattgccgcacgtgatctgatttgtgcacagaaatttaacggcctgaccgttctgcctccgctgctgaccgatgaaatgattgcacagtataccagcgcactgctggcaggcaccattaccagtggttggacctttggtgccggtgccgcactgcagattccgtttgcaatgcagatggcatatcgttttaatggtattggtgttacccagaacgtgctgtatgaaaaccagaaactgattgccaaccagtttaatagcgccattggcaaaattcaggatagcctgagcagcaccgcaagtgcactgggtaaactgcaggacgttgttaatcagaatgcacaggcactgaataccctggttaaacagctgagcagtaattttggtgcaatttcaagcgtgctgaacgatattctgagccgtctggataaagttgaagcagaagttcagattgatcgtctgattaccggtcgtctgcaaagcctgcagacctatgtgacccagcagctgattcgcgcagcagaaattcgtgcaagcgcaaatctggcagccaccaaaatgagcgaatgtgttctgggtcagagcaaacgtgttgatttttgcggcaaaggttatcacctgatgagctttccgcagagcgcaccgcatggtgttgtgtttctgcatgttacctatgttccggcacaagaaaaaaactttacaaccgctccggcaatttgccatgatggtaaagcacattttccgcgtgaaggtgtttttgttagtaatggcacccattggtttgttacacagcgcaacttttatgaaccgcagattattacaaccgacaacacctttgttagcggtaactgtgatgttgtgattggcattgtgaataacaccgtttatgatccactgcagccggaactggatagctttaaagaagaactggacaaatatttcaaaaaccacaccagtccggatgttgatctgggtgatatttcaggtattaatgccagcgtggtgaacatccagaaagaaattgatcgcctgaatgaagtggccaaaaatctgaatgaaagcctgattgatctgcaagaactggggaaatatgagcagtatatcaaatggccgtggtatatttggctgggttttattgcaggcctgattgcaattgttatggtgaccattatgctgtgttgtatgaccagctgttgtagctgtctgaaaggttgttgcagctgcggtagctgttgcaaatttgatgaagatgatagcgaaccggtgctgaaaggtgttaaactgcattatacctaatgagcggccgc

SEQ ID NO: 3 - DNA, 4556 bp, Artificial Sequence
  nucleic acid encoding for fusion protein HEV- SARS-CoV-2 spike protein- optimised for expression in E. coli
gagctcatgattgcactgaccctgtttaatctggcagataccctgttaggtggtctgccgaccgaactgattagcagtgccggtggtcagctgttttatagccgtccggttgttagcgcaaatggtgaaccgaccgttaaactgtataccagcgttgaaaatgcacagcaggataaaggtattgcaattccgcatgatattgatctgggtgaaagccgtgttgtgattcaggattatgataatcagcatgaacaggatcgtccgacaccgagtccggcaccgagccgtccgtttagcgttctgcgtgcaaatgatgttctgtggctgagcctgaccgcagcagaatatgatcagagcacctatggtagcagcaccggtccggtttatgttagcgatagcgttaccctggttaatgttgcaaccggtgcacaggcagttgcacgtagcctggattggaccaaagtgaccctggatggtcgtccgctgagcaccattcagcagtatagcaaaaccttttttgttctgccgctgcgtggtaaactgagcttttgggaagcaggcaccaccaaagcaggttatccgtataactataataccaccgcaagcgatcagctgctggttgaaaacgcagcaggtcatcgtgttgcaattagcacctataccaccagtttaggtgcaggtccggttagcattagcgcagttgcagttctggcaccgcatagcgcatttgtttttctggttctgctgccgctggttagcagccagtgtgttaatctgaccacacgtacccagctgcctccggcatataccaatagctttacccgtggtgtttattatccggacaaagtttttcgtagcagcgttctgcatagcacccaggacctgtttctgccgttttttagcaatgttacctggtttcatgccattcatgttagcggcaccaatggcaccaaacgttttgataatccggtgctgccgtttaatgatggtgtgtattttgcaagcaccgaaaaaagcaacattattcgcggttggatttttggtacaaccctggatagcaaaacccagagcctgctgattgttaataatgccaccaatgtggtgatcaaagtgtgcgaatttcagttttgcaatgatccgtttctgggcgtgtattaccacaaaaataacaagagctggatggaaagcgaatttcgtgtttatagcagcgccaataattgcacctttgaatatgttagccagccgtttctgatggatctggaaggtaaacagggtaactttaaaaacctgcgcgagttcgtgttcaaaaacatcgatggttacttcaaaatctatagcaaacacaccccgattaatctggttcgtgatctgccgcagggttttagcgcactggaaccgctggttgatctgccaattggtattaacattacccgttttcagaccctgctggcactgcatcgtagctatctgacaccgggtgatagcagcagcggttggaccgcaggcgcagcagcatattatgttggttatctgcagcctcgtacctttctgctgaaatataacgaaaatggcacaattaccgatgccgttgattgtgccctggatccgctgagcgaaaccaaatgtaccctgaaaagctttaccgttgagaaaggtatttatcagaccagcaattttcgtgtgcagccgaccgaaagcattgttcgttttccgaatatcaccaatctgtgtccgtttggcgaagtttttaatgcaacccgttttgccagcgtttatgcatggaatcgtaaacgtattagcaattgcgttgccgattatagcgttctgtataatagcgcaagcttcagcacctttaaatgctatggtgttagcccgaccaaactgaatgatctgtgttttaccaatgtgtatgccgatagctttgtgattcgtggtgatgaagttcgtcagattgcaccgggtcagaccggtaaaattgcagattataactataaactgccggatgattttacgggttgtgttattgcctggaatagcaataatctggacagcaaagttggtggcaactataactatctgtatcgcctgtttcgtaagagcaatctgaaaccgtttgaacgtgatattagcaccgagatttatcaggcaggtagcaccccgtgtaatggtgttgaaggttttaattgctattttccgctgcagagctatggttttcagccgacaaatggtgtgggttatcagccgtatcgtgttgttgttctgtcatttgaactgctgcatgcaccggcaaccgtttgtggtccgaaaaaaagtaccaatctggtgaaaaataagtgcgtgaactttaactttaatggtctgaccggcaccggtgttctgaccgaaagtaacaaaaaattcctgccgtttcagcagtttggccgtgatattgcagataccaccgatgcagttcgcgatccgcagacactggaaattctggatattaccccgtgcagctttggtggtgtttcagttattacaccgggtacaaataccagcaatcaggttgcagttctgtatcaggatgttaattgtaccgaagttccggttgcaattcatgcagatcagctgaccccgacctggcgtgtgtatagcaccggtagcaatgtgtttcagacacgtgcaggttgtctgattggtgcagaacatgtgaataatagctatgaatgcgatattccgattggtgcgggtatttgtgccagctatcagacccagaccaatagtccgcgtcgtgcacgtagcgttgcaagccagagcattattgcctataccatgagcctgggtgcagaaaatagcgttgcctatagtaataacagcattgccattccgaccaactttaccattagcgttaccaccgaaattctgccggttagcatgaccaaaaccagcgttgattgcaccatgtatatttgtggtgatagtaccgaatgtagcaatctgctgctgcagtatggtagcttttgcacccagctgaatcgtgcactgaccggtattgcagttgaacaggataaaaacacgcaagaagtttttgcacaggtcaagcagatctataaaacccctccgattaaagattttggcggtttcaattttagccagatcctgccggatccgagcaaaccgagtaaacgtagctttattgaagatctgctgttcaacaaagtgaccctggcagatgcaggttttatcaaacagtatggtgattgcctgggcgatattgccgcacgtgatctgatttgtgcacagaaatttaacggcctgaccgttctgcctccgctgctgaccgatgaaatgattgcacagtataccagcgcactgctggcaggcaccattaccagtggttggacctttggtgccggtgccgcactgcagattccgtttgcaatgcagatggcatatcgttttaatggtattggtgttacccagaacgtgctgtatgaaaaccagaaactgattgccaaccagtttaatagcgccattggcaaaattcaggatagcctgagcagcaccgcaagtgcactgggtaaactgcaggacgttgttaatcagaatgcacaggcactgaataccctggttaaacagctgagcagtaattttggtgcaatttcaagcgtgctgaacgatattctgagccgtctggataaagttgaagcagaagttcagattgatcgtctgattaccggtcgtctgcaaagcctgcagacctatgtgacccagcagctgattcgcgcagcagaaattcgtgcaagcgcaaatctggcagccaccaaaatgagcgaatgtgttctgggtcagagcaaacgtgttgatttttgcggcaaaggttatcacctgatgagctttccgcagagcgcaccgcatggtgttgtgtttctgcatgttacctatgttccggcacaagaaaaaaactttacaaccgctccggcaatttgccatgatggtaaagcacattttccgcgtgaaggtgtttttgttagtaatggcacccattggtttgttacacagcgcaacttttatgaaccgcagattattacaaccgacaacacctttgttagcggtaactgtgatgttgtgattggcattgtgaataacaccgtttatgatccactgcagccggaactggatagctttaaagaagaactggacaaatatttcaaaaaccacaccagtccggatgttgatctgggtgatatttcaggtattaatgccagcgtggtgaacatccagaaagaaattgatcgcctgaatgaagtggccaaaaatctgaatgaaagcctgattgatctgcaagaactggggaaatatgagcagtatatcaaatggccgtggtatatttggctgggttttattgcaggcctgattgcaattgttatggtgaccattatgctgtgttgtatgaccagctgttgtagctgtctgaaaggttgttgcagctgcggtagctgttgcaaatttgatgaagatgatagcgaaccggtgctgaaaggtgttaaactgcattatacctaatgagcggccgc

SEQ ID NO: 4 - DNA, 3839 bp, Artificial Sequence
  SARS-CoV-2 spike protein nucleic acid sequence - optimised for expression in Komagataella pastoris
ttcgaaacgatgttcgtgttcttggtcctgttgccattggtttcttcccagtgtgttaacctgaccactagaactcaattgcctccagcctacaccaattccttcaccagaggtgtttactacccagacaaggtgttcagatcttccgtcttgcactccactcaggacttgttcttgccattcttctccaacgttacctggttccacgctattcacgtttccggaactaacggtactaagagattcgacaacccagtcctgccattcaacgatggtgtctacttcgcttctaccgagaagtccaacatcatcagaggttggatcttcggtactaccctggactctaagactcagtccttgctgatcgttaacaacgccaccaacgttgtcatcaaggtttgcgagttccagttctgcaacgacccattcttgggtgtgtactaccacaagaacaacaagtcttggatggaatccgagttcagagtttactcctccgccaacaactgtaccttcgagtacgtttcccagccattcttgatggacttggagggtaagcagggtaacttcaagaacctgagagagttcgttttcaagaacatcgacggttacttcaagatctactccaagcacaccccaatcaacctggttagagatttgccacaaggtttctccgctttggagcctttggttgacttgccaatcggtatcaacatcaccagattccagaccttgttggccttgcacagatcctacttgactccaggtgattcttcttccggttggactgctggtgctgctgcttactatgttggttacttgcagccaagaaccttcctgctgaagtacaacgagaacggaactatcactgacgctgttgactgtgctttggacccattgtctgagactaagtgcaccttgaagtccttcaccgttgagaagggtatctaccagacctccaacttcagagttcagccaactgagtccatcgtcagattcccaaacatcactaacttgtgcccattcggtgaggtgttcaacgctactagattcgcttctgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacctgtaccgtctgttcagaaagtccaacttgaagccattcgagagagacatctccaccgagatctaccaagctggttctactccatgtaacggtgtcgagggtttcaactgctacttcccattgcaatcctacggtttccaacctaccaacggtgttggataccagccatacagagttgtcgttttgtccttcgagttgttgcacgctccagctactgtttgtggtccaaagaagtccaccaacttggtcaagaacaaatgcgtcaactttaacttcaacggcctgaccggtactggtgttttgactgaatccaacaagaagttcctgcctttccagcagttcggtagagacattgctgacactactgacgccgttagagatccacagactttggagatcttggacatcaccccatgttccttcggtggtgtttccgttattacccctggaactaacacctccaatcaggtcgctgtcttgtaccaggacgttaactgtactgaggttccagttgctatccacgctgaccaattgactccaacttggagagtctactccaccggttccaacgttttccaaactagagccggttgtttgatcggtgctgaacacgtcaacaactcctacgagtgtgacattccaattggtgctggtatctgtgcctcctaccaaactcaaactaactccccaagaagggctagatccgttgcttcccaatccattatcgcttacaccatgtctttgggtgccgagaactctgttgcctactctaacaactctatcgctatccctaccaacttcaccatctccgttaccactgagatcttgccagtctccatgaccaagacttccgttgactgtaccatgtacatctgtggtgactccactgagtgttccaacttgttgctgcaatacggttccttctgcacccagttgaacagagctttgactggtattgctgtcgagcaagacaagaacactcaagaggttttcgcccaggtgaagcagatctacaagactccacctattaaggacttcggtggcttcaacttctcccagattttgccagatccatctaagccctccaagagatccttcattgaggacctgctgttcaacaaggttactttggctgacgccggtttcatcaagcagtacggtgattgcttgggtgacattgcagctagagacttgatctgtgcccagaagttcaacggtttgaccgttttgccacctttgttgaccgacgagatgatcgctcagtacacttctgctttgttggccggtactatcacttctggttggacatttggagctggtgccgcattgcaaattccattcgctatgcaaatggcctacagattcaacggtatcggtgttacccagaacgtcctgtacgagaaccagaagcttatcgccaaccagttcaactccgctatcggtaagattcaggactccttgtcctctactgcttctgccttgggaaagttgcaggatgttgttaaccagaatgcccaggctttgaacaccctggttaagcaactgtcctctaacttcggtgctatctcctccgttttgaacgacatcttgtcccgtttggacaaggttgaggctgaggttcagatcgacagattgatcactggtagattgcagtccctgcagacttacgttactcagcagttgattagagctgccgagattagagcctctgctaacttggctgctactaagatgtccgagtgtgttttgggtcagtccaagagagttgacttctgcggtaagggttaccacctgatgtctttcccacaatctgctccacacggtgtcgttttcttgcacgttacttacgttccagctcaagagaagaacttcactactgctccagccatttgtcacgatggtaaggctcactttcctcgtgagggtgttttcgtttccaacggtactcactggttcgtcacccagagaaacttttacgagccacagatcatcaccaccgacaacactttcgtttctggtaactgtgacgtcgtcatcggtatcgtgaacaacactgtctacgatccattgcagccagaattggactccttcaaagaggaactggacaagtactttaagaaccacacttccccagacgttgacctgggtgatatttccggtattaacgcctccgttgtcaacatccaaaaagagatcgaccgtttgaacgaggtcgccaagaacttgaacgagtccttgattgacttgcaagagctgggcaagtacgagcagtacattaagtggccatggtacatttggctgggtttcattgctggtttgatcgccatcgttatggtcaccatcatgttgtgctgtatgacctcctgttgctcctgtttgaagggttgttgttcctgcggttcctgttgtaagttcgacgaagatgactccgagccagtcttgaagggtgttaagttgcactacacttaagcggccgc

SEQ ID NO: 5 - DNA, 5357 bp, Artificial Sequence
  nucleic acid encoding for fusion protein HPV18L1/SARS-CoV-2 spike protein- optimised for expression in K. pastoris
ttcgaaacgatggctctttggagaccatccgacaacactgtttacttgccaccaccatccgttgctagagttgttaacactgacgactacgttactagaacttccatcttctaccacgctggttcttccagattgttgactgttggtaacccatacttcagagttccagctggaggtggtaacaagcaagacatcccaaaggtttccgcttaccagtacagagttttcagagttcagttgccagacccaaacaagtttggattgccagacacttccatctacaacccagagactcagagacttgtttgggcttgtgctggtgttgaaatcggtagaggacagccattgggtgttggtttgtctggtcacccattctacaacaagttggacgacactgaatcttctcacgctgctacttctaacgtttccgaggatgttagagacaacgtttccgttgactacaagcagactcagttgtgtatcttgggttgtgctccagctattggtgaacattgggctaagggtactgcttgtaagtccagaccattgtctcagggagattgtccaccattggagttgaagaacactgttttggaggacggtgatatggttgatactggttacggtgctatggacttctctactttgcaggacactaagtgtgaagttccattggacatctgtcagtccatctgtaagtacccagactacttgcaaatgtccgctgatccatacggtgactctatgttcttctgtttgagaagagagcagttgttcgctagacacttctggaacagagctggtactatgggtgacactgttccacaatccttgtacatcaagggtactggaatgagagcttctcctggttcttgtgtttactctccatctccatccggttccattgttacttccgactcccagttgttcaacaagccatactggttgcataaggctcaaggtcacaacaacggtgtttgttggcacaaccagttgttcgttactgttgttgacactactagatccactaacttgactatctgtgcttccactcaatctccagttccaggacaatacgacgctactaagttcaagcagtactccagacacgttgaagagtacgacttgcagttcatcttccagttgtgtactatcactttgactgctgatgttatgtcctacatccactctatgaactcctccattttggaggattggaacttcggtgttccaccaccaccaactacttcattggttgacacttacagattcgttcagtccgttgctatcacttgtcaaaaggacgctgctccagctgaaaacaaggacccatacgacaagttgaagttctggaacgttgacttgaaagagaagttctccttggacttggaccaatacccattgggtagaaagtttttggttcaggctggattgagaagaaagccaactatcggtccaagaaagagatcagctccatccgctactacttcatccaagccagctaagagagttagagttagagctagaaagttcgtgttcttggtcctgttgccattggtttcttcccagtgtgttaacctgaccactagaactcaattgcctccagcctacaccaattccttcaccagaggtgtttactacccagacaaggtgttcagatcttccgtcttgcactccactcaggacttgttcttgccattcttctccaacgttacctggttccacgctattcacgtttccggaactaacggtactaagagattcgacaacccagtcctgccattcaacgatggtgtctacttcgcttctaccgagaagtccaacatcatcagaggttggatcttcggtactaccctggactctaagactcagtccttgctgatcgttaacaacgccaccaacgttgtcatcaaggtttgcgagttccagttctgcaacgacccattcttgggtgtgtactaccacaagaacaacaagtcttggatggaatccgagttcagagtttactcctccgccaacaactgtaccttcgagtacgtttcccagccattcttgatggacttggagggtaagcagggtaacttcaagaacctgagagagttcgttttcaagaacatcgacggttacttcaagatctactccaagcacaccccaatcaacctggttagagatttgccacaaggtttctccgctttggagcctttggttgacttgccaatcggtatcaacatcaccagattccagaccttgttggccttgcacagatcctacttgactccaggtgattcttcttccggttggactgctggtgctgctgcttactatgttggttacttgcagccaagaaccttcctgctgaagtacaacgagaacggaactatcactgacgctgttgactgtgctttggacccattgtctgagactaagtgcaccttgaagtccttcaccgttgagaagggtatctaccagacctccaacttcagagttcagccaactgagtccatcgtcagattcccaaacatcactaacttgtgcccattcggtgaggtgttcaacgctactagattcgcttctgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacctgtaccgtctgttcagaaagtccaacttgaagccattcgagagagacatctccaccgagatctaccaagctggttctactccatgtaacggtgtcgagggtttcaactgctacttcccattgcaatcctacggtttccaacctaccaacggtgttggataccagccatacagagttgtcgttttgtccttcgagttgttgcacgctccagctactgtttgtggtccaaagaagtccaccaacttggtcaagaacaaatgcgtcaactttaacttcaacggcctgaccggtactggtgttttgactgaatccaacaagaagttcctgcctttccagcagttcggtagagacattgctgacactactgacgccgttagagatccacagactttggagatcttggacatcaccccatgttccttcggtggtgtttccgttattacccctggaactaacacctccaatcaggtcgctgtcttgtaccaggacgttaactgtactgaggttccagttgctatccacgctgaccaattgactccaacttggagagtctactccaccggttccaacgttttccaaactagagccggttgtttgatcggtgctgaacacgtcaacaactcctacgagtgtgacattccaattggtgctggtatctgtgcctcctaccaaactcaaactaactccccaagaagggctagatccgttgcttcccaatccattatcgcttacaccatgtctttgggtgccgagaactctgttgcctactctaacaactctatcgctatccctaccaacttcaccatctccgttaccactgagatcttgccagtctccatgaccaagacttccgttgactgtaccatgtacatctgtggtgactccactgagtgttccaacttgttgctgcaatacggttccttctgcacccagttgaacagagctttgactggtattgctgtcgagcaagacaagaacactcaagaggttttcgcccaggtgaagcagatctacaagactccacctattaaggacttcggtggcttcaacttctcccagattttgccagatccatctaagccctccaagagatccttcattgaggacctgctgttcaacaaggttactttggctgacgccggtttcatcaagcagtacggtgattgcttgggtgacattgcagctagagacttgatctgtgcccagaagttcaacggtttgaccgttttgccacctttgttgaccgacgagatgatcgctcagtacacttctgctttgttggccggtactatcacttctggttggacatttggagctggtgccgcattgcaaattccattcgctatgcaaatggcctacagattcaacggtatcggtgttacccagaacgtcctgtacgagaaccagaagcttatcgccaaccagttcaactccgctatcggtaagattcaggactccttgtcctctactgcttctgccttgggaaagttgcaggatgttgttaaccagaatgcccaggctttgaacaccctggttaagcaactgtcctctaacttcggtgctatctcctccgttttgaacgacatcttgtcccgtttggacaaggttgaggctgaggttcagatcgacagattgatcactggtagattgcagtccctgcagacttacgttactcagcagttgattagagctgccgagattagagcctctgctaacttggctgctactaagatgtccgagtgtgttttgggtcagtccaagagagttgacttctgcggtaagggttaccacctgatgtctttcccacaatctgctccacacggtgtcgttttcttgcacgttacttacgttccagctcaagagaagaacttcactactgctccagccatttgtcacgatggtaaggctcactttcctcgtgagggtgttttcgtttccaacggtactcactggttcgtcacccagagaaacttttacgagccacagatcatcaccaccgacaacactttcgtttctggtaactgtgacgtcgtcatcggtatcgtgaacaacactgtctacgatccattgcagccagaattggactccttcaaagaggaactggacaagtactttaagaaccacacttccccagacgttgacctgggtgatatttccggtattaacgcctccgttgtcaacatccaaaaagagatcgaccgtttgaacgaggtcgccaagaacttgaacgagtccttgattgacttgcaagagctgggcaagtacgagcagtacattaagtggccatggtacatttggctgggtttcattgctggtttgatcgccatcgttatggtcaccatcatgttgtgctgtatgacctcctgttgctcctgtttgaagggttgttgttcctgcggttcctgttgtaagttcgacgaagatgactccgagccagtcttgaagggtgttaagttgcactacacttaagcggccgc

SEQ ID NO: 6 - DNA, 5351 bp, Artificial Sequence
  nucleic acid encoding for fusion protein HPV16L1/SARS-CoV-2 spike protein - optimised for expression in K. pastoris
ttcgaaacgatgtctttgtggttgccatctgaagctactgtttacttgccaccagttccagtttctaaagttgtttccactgacgaatacgttgctagaactaacatctactaccacgctggtacttctagattgttggctgttggtcatccatacttcccaattaagaagccaaacaacaacaagattttggttccaaaggtttccggattgcaatacagagttttcagaatccatttgccagatccaaacaagtttggtttcccagatacttctttctacaacccagacactcaaagacttgtttgggcttgtgttggtgttgaagttggtagaggtcaaccattgggtgttggtatttctggtcacccattgttgaacaagttggacgatactgaaaacgcttctgcttacgctgctaacgctggtgttgataacagagaatgtatttctatggactacaagcaaactcaattgtgtttgattggttgtaagccaccaattggtgaacattggggaaagggttctccatgtactaatgttgctgttaaccctggtgattgtccaccattggaattgattaacactgttattcaagacggtgatatggttgatactggtttcggtgctatggatttcactactttgcaagctaacaagtctgaagttccattggacatttgtacttccatctgtaagtacccagactacattaagatggtttctgaaccatacggtgattctttgttcttctacttgagaagagaacaaatgtttgttagacacttgttcaacagagctggtgctgttggtgaaaacgttccagatgacttgtacattaagggttctggttctactgctaacttggcttcttctaactactttccaactccatctggttctatggttacttctgacgctcaaattttcaacaagccatactggttgcaaagagcacaaggtcataacaacggtatttgttggggtaaccaattgttcgttactgttgttgacactactagatccactaacatgtccttgtgtgctgctatttctacttctgaaactacttacaagaacactaacttcaaagagtacttgagacacggagaagaatacgacttgcaattcattttccaattgtgtaagattactttgactgctgacgttatgacttacattcactctatgaactctactattttggaagattggaacttcggattgcaaccaccaccaggtggtactttggaagatacttacagattcgttacttctcaagctattgcttgtcaaaagcatactccacctgctccaaaagaagatccattgaagaagtacactttctgggaagttaacttgaaagaaaagttctctgctgatttggatcaattcccattgggtagaaagtttttgttgcaagctggattgaaggctaaaccaaagttcactttgggaaagagaaaggctactccaactacttcttctacttctactactgctaagagaaagaagagaaaattgttcgtgttcttggtcctgttgccattggtttcttcccagtgtgttaacctgaccactagaactcaattgcctccagcctacaccaattccttcaccagaggtgtttactacccagacaaggtgttcagatcttccgtcttgcactccactcaggacttgttcttgccattcttctccaacgttacctggttccacgctattcacgtttccggaactaacggtactaagagattcgacaacccagtcctgccattcaacgatggtgtctacttcgcttctaccgagaagtccaacatcatcagaggttggatcttcggtactaccctggactctaagactcagtccttgctgatcgttaacaacgccaccaacgttgtcatcaaggtttgcgagttccagttctgcaacgacccattcttgggtgtgtactaccacaagaacaacaagtcttggatggaatccgagttcagagtttactcctccgccaacaactgtaccttcgagtacgtttcccagccattcttgatggacttggagggtaagcagggtaacttcaagaacctgagagagttcgttttcaagaacatcgacggttacttcaagatctactccaagcacaccccaatcaacctggttagagatttgccacaaggtttctccgctttggagcctttggttgacttgccaatcggtatcaacatcaccagattccagaccttgttggccttgcacagatcctacttgactccaggtgattcttcttccggttggactgctggtgctgctgcttactatgttggttacttgcagccaagaaccttcctgctgaagtacaacgagaacggaactatcactgacgctgttgactgtgctttggacccattgtctgagactaagtgcaccttgaagtccttcaccgttgagaagggtatctaccagacctccaacttcagagttcagccaactgagtccatcgtcagattcccaaacatcactaacttgtgcccattcggtgaggtgttcaacgctactagattcgcttctgtttacgcctggaacagaaagagaatctccaactgcgttgctgactactccgtcttgtacaactctgcttcattctccaccttcaagtgctacggtgtttccccaactaagttgaacgacctgtgtttcactaacgtctacgccgactccttcgttattagaggtgacgaggttagacagatcgctccaggtcaaactggtaagatcgctgactacaactacaagctgccagacgacttcaccggttgtgttattgcttggaactccaacaacctggactccaaggttggtggtaactacaattacctgtaccgtctgttcagaaagtccaacttgaagccattcgagagagacatctccaccgagatctaccaagctggttctactccatgtaacggtgtcgagggtttcaactgctacttcccattgcaatcctacggtttccaacctaccaacggtgttggataccagccatacagagttgtcgttttgtccttcgagttgttgcacgctccagctactgtttgtggtccaaagaagtccaccaacttggtcaagaacaaatgcgtcaactttaacttcaacggcctgaccggtactggtgttttgactgaatccaacaagaagttcctgcctttccagcagttcggtagagacattgctgacactactgacgccgttagagatccacagactttggagatcttggacatcaccccatgttccttcggtggtgtttccgttattacccctggaactaacacctccaatcaggtcgctgtcttgtaccaggacgttaactgtactgaggttccagttgctatccacgctgaccaattgactccaacttggagagtctactccaccggttccaacgttttccaaactagagccggttgtttgatcggtgctgaacacgtcaacaactcctacgagtgtgacattccaattggtgctggtatctgtgcctcctaccaaactcaaactaactccccaagaagggctagatccgttgcttcccaatccattatcgcttacaccatgtctttgggtgccgagaactctgttgcctactctaacaactctatcgctatccctaccaacttcaccatctccgttaccactgagatcttgccagtctccatgaccaagacttccgttgactgtaccatgtacatctgtggtgactccactgagtgttccaacttgttgctgcaatacggttccttctgcacccagttgaacagagctttgactggtattgctgtcgagcaagacaagaacactcaagaggttttcgcccaggtgaagcagatctacaagactccacctattaaggacttcggtggcttcaacttctcccagattttgccagatccatctaagccctccaagagatccttcattgaggacctgctgttcaacaaggttactttggctgacgccggtttcatcaagcagtacggtgattgcttgggtgacattgcagctagagacttgatctgtgcccagaagttcaacggtttgaccgttttgccacctttgttgaccgacgagatgatcgctcagtacacttctgctttgttggccggtactatcacttctggttggacatttggagctggtgccgcattgcaaattccattcgctatgcaaatggcctacagattcaacggtatcggtgttacccagaacgtcctgtacgagaaccagaagcttatcgccaaccagttcaactccgctatcggtaagattcaggactccttgtcctctactgcttctgccttgggaaagttgcaggatgttgttaaccagaatgcccaggctttgaacaccctggttaagcaactgtcctctaacttcggtgctatctcctccgttttgaacgacatcttgtcccgtttggacaaggttgaggctgaggttcagatcgacagattgatcactggtagattgcagtccctgcagacttacgttactcagcagttgattagagctgccgagattagagcctctgctaacttggctgctactaagatgtccgagtgtgttttgggtcagtccaagagagttgacttctgcggtaagggttaccacctgatgtctttcccacaatctgctccacacggtgtcgttttcttgcacgttacttacgttccagctcaagagaagaacttcactactgctccagccatttgtcacgatggtaaggctcactttcctcgtgagggtgttttcgtttccaacggtactcactggttcgtcacccagagaaacttttacgagccacagatcatcaccaccgacaacactttcgtttctggtaactgtgacgtcgtcatcggtatcgtgaacaacactgtctacgatccattgcagccagaattggactccttcaaagaggaactggacaagtactttaagaaccacacttccccagacgttgacctgggtgatatttccggtattaacgcctccgttgtcaacatccaaaaagagatcgaccgtttgaacgaggtcgccaagaacttgaacgagtccttgattgacttgcaagagctgggcaagtacgagcagtacattaagtggccatggtacatttggctgggtttcattgctggtttgatcgccatcgttatggtcaccatcatgttgtgctgtatgacctcctgttgctcctgtttgaagggttgttgttcctgcggttcctgttgtaagttcgacgaagatgactccgagccagtcttgaagggtgttaagttgcactacacttaagcggccgc

SEQ ID NO: 7 - DNA, 3836 bp, Artificial Sequence
  SARS-CoV-2 spike protein nucleic acid sequence - optimised for expression in humans
gctagcgacatgttcgtgtttctggtgctgctgcctctggtgtccagccagtgtgtgaacctgaccaccagaacacagctgcctccagcctacaccaatagcttcaccaggggcgtgtactaccccgacaaggtgttcagatctagcgtgctgcacagcacccaggacctgtttctgcccttcttcagcaacgtgacctggttccacgccatccacgtgtccggcaccaatggcaccaagagattcgacaaccccgtgctgcccttcaacgatggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgacccattcctgggagtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttttctgctctggaacctctggtggacctgcctatcggcatcaacatcacccggtttcagaccctgctggccctgcacagatcttacctgacacctggcgatagcagctctggatggacagctggcgccgctgcctattatgtgggctacctgcagcctcggaccttcctgctgaagtacaacgagaacggcaccatcaccgacgccgtggattgtgctctggatcccctgagcgagacaaagtgcaccctgaagtccttcaccgtggaaaagggcatctaccagaccagcaacttcagagtgcagcccaccgagagcatcgtgcggttccccaatatcaccaatctgtgccccttcggcgaggtgttcaatgccacaagatttgccagcgtgtacgcctggaaccggaagagaatcagcaactgcgtggccgactacagcgtgctgtacaatagcgccagcttcagcaccttcaagtgctacggcgtgtcccctaccaagctgaacgacctgtgcttcaccaatgtgtacgccgacagcttcgtgatcagaggcgacgaagttcggcagatcgctcctggacagacaggcaagatcgccgattacaactacaagctgcccgacgacttcaccggctgcgtgatcgcctggaatagcaacaacctggactccaaagtcggcggcaactacaactacctgtaccggctgttccggaagtccaatctgaagcccttcgagcgggacatctccaccgaaatctatcaggccggcagcaccccttgtaacggcgtggaaggcttcaactgctacttcccactgcagtcctacggctttcagcctaccaatggcgtgggctatcagccctatagagtggtggtgctgagcttcgaactgctgcatgcccctgctaccgtgtgcggccctaagaagtctaccaacctggtcaagaacaaatgcgtgaacttcaacttcaacggcctgaccggcacaggcgtgctgacagagagcaacaagaagttcctgcctttccagcagtttggccgggatatcgccgataccacagacgccgttagagatccccagacactggaaatcctggacatcaccccatgcagctttggcggagtgtctgtgatcacccctggcaccaataccagcaatcaggtggccgtgctgtatcaggacgtgaactgtacagaggtgcccgtggccattcacgccgatcaactgacacccacttggagagtgtactccaccggctccaacgtgttccagactagagccggatgtctgatcggagccgagcacgtgaacaatagctacgagtgcgacatccccatcggcgctggcatctgtgccagctaccagacacagacaaatagccccagacgggccagaagcgtggcctctcagagcatcattgcctacacaatgagcctgggcgccgagaattctgtggcctacagcaacaactctatcgctatccccaccaacttcaccatcagcgtgaccaccgagatcctgcctgtgtccatgaccaagaccagcgtggactgcaccatgtacatctgcggcgattccaccgagtgcagcaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagctttatcgaggacctgctgttcaacaaagtgacactggccgacgccggcttcatcaagcagtatggcgattgcctgggcgacattgccgccagagatctgatttgcgcccagaagtttaacggactgacagtgctgcctcctctgctgaccgatgagatgatcgcccagtacacatctgctctgctggccggcacaatcaccagcggatggacatttggagctggcgcagccctgcagatcccctttgctatgcagatggcctaccggttcaacggcatcggagtgacccagaatgtgctgtacgagaaccagaagctgatcgccaaccagttcaacagcgccatcggcaagatccaggatagcctgtctagcacagccagcgctctgggcaaactgcaggacgtggtcaatcagaacgctcaggccctgaacaccctcgtgaagcagctgagcagcaatttcggcgccatcagctccgtgctgaacgatatcctgagccggctggataaggtggaagccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggatttctgcggcaagggctaccacctgatgagctttccacagtctgctcctcacggcgtggtgtttctgcacgtgacctatgtgcccgctcaagagaagaacttcacaacagcccctgccatctgccacgacggaaaggcccattttcctagagaaggcgtgttcgtgtccaacggcacccattggttcgtgacacagcggaacttctacgagccccagatcatcaccaccgacaacaccttcgtgtctggcaactgtgacgtcgtgatcggcattgtgaacaacaccgtgtacgaccctctgcagcccgagctggacagcttcaaagaggaactggacaagtactttaagaaccacacaagccccgacgtggacctgggcgatattagcggcatcaatgcctccgtggtcaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgctgcatgaccagctgctgtagctgcctgaagggctgttgcagctgtggcagctgctgcaagttcgacgaggatgatagcgagcctgtgctgaagggcgtgaaactgcactacaccgcggccgc

SEQ ID NO: 8 - DNA, 4232 bp, Artificial Sequence
  nucleic acid encoding for fusion protein HBSAg/ SARS-CoV-2 spike protein- optimised for expression in humans
gctagcgacatgaactttctgggcggtacgacagtatgccttggacaaaattcacaatctccgacgtctaatcactcccctacaagttgtccaccgacttgccccggctataggtggatgtgtctcagacgattcataatctttctcttcattcttcttctgtgcctgatattcttgctggtccttctggattaccagggaatgcttcccgtgtgtcctctgattcctggttcatccactacatctacgggtccctgtagaacatgcaccacacctgcacagggcacctccatgtatccgtcatgctgctgcacgaaaccatcagatggtaactgcacgtgcataccgatcccctcatcatgggcgtttgggaaatttctgtgggagtgggcctcagcccggttttccttcgtgtttctggtgctgctgcctctggtgtccagccagtgtgtgaacctgaccaccagaacacagctgcctccagcctacaccaatagcttcaccaggggcgtgtactaccccgacaaggtgttcagatctagcgtgctgcacagcacccaggacctgtttctgcccttcttcagcaacgtgacctggttccacgccatccacgtgtccggcaccaatggcaccaagagattcgacaaccccgtgctgcccttcaacgatggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgacccattcctgggagtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttttctgctctggaacctctggtggacctgcctatcggcatcaacatcacccggtttcagaccctgctggccctgcacagatcttacctgacacctggcgatagcagctctggatggacagctggcgccgctgcctattatgtgggctacctgcagcctcggaccttcctgctgaagtacaacgagaacggcaccatcaccgacgccgtggattgtgctctggatcccctgagcgagacaaagtgcaccctgaagtccttcaccgtggaaaagggcatctaccagaccagcaacttcagagtgcagcccaccgagagcatcgtgcggttccccaatatcaccaatctgtgccccttcggcgaggtgttcaatgccacaagatttgccagcgtgtacgcctggaaccggaagagaatcagcaactgcgtggccgactacagcgtgctgtacaatagcgccagcttcagcaccttcaagtgctacggcgtgtcccctaccaagctgaacgacctgtgcttcaccaatgtgtacgccgacagcttcgtgatcagaggcgacgaagttcggcagatcgctcctggacagacaggcaagatcgccgattacaactacaagctgcccgacgacttcaccggctgcgtgatcgcctggaatagcaacaacctggactccaaagtcggcggcaactacaactacctgtaccggctgttccggaagtccaatctgaagcccttcgagcgggacatctccaccgaaatctatcaggccggcagcaccccttgtaacggcgtggaaggcttcaactgctacttcccactgcagtcctacggctttcagcctaccaatggcgtgggctatcagccctatagagtggtggtgctgagcttcgaactgctgcatgcccctgctaccgtgtgcggccctaagaagtctaccaacctggtcaagaacaaatgcgtgaacttcaacttcaacggcctgaccggcacaggcgtgctgacagagagcaacaagaagttcctgcctttccagcagtttggccgggatatcgccgataccacagacgccgttagagatccccagacactggaaatcctggacatcaccccatgcagctttggcggagtgtctgtgatcacccctggcaccaataccagcaatcaggtggccgtgctgtatcaggacgtgaactgtacagaggtgcccgtggccattcacgccgatcaactgacacccacttggagagtgtactccaccggctccaacgtgttccagactagagccggatgtctgatcggagccgagcacgtgaacaatagctacgagtgcgacatccccatcggcgctggcatctgtgccagctaccagacacagacaaatagccccagacgggccagaagcgtggcctctcagagcatcattgcctacacaatgagcctgggcgccgagaattctgtggcctacagcaacaactctatcgctatccccaccaacttcaccatcagcgtgaccaccgagatcctgcctgtgtccatgaccaagaccagcgtggactgcaccatgtacatctgcggcgattccaccgagtgcagcaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagctttatcgaggacctgctgttcaacaaagtgacactggccgacgccggcttcatcaagcagtatggcgattgcctgggcgacattgccgccagagatctgatttgcgcccagaagtttaacggactgacagtgctgcctcctctgctgaccgatgagatgatcgcccagtacacatctgctctgctggccggcacaatcaccagcggatggacatttggagctggcgcagccctgcagatcccctttgctatgcagatggcctaccggttcaacggcatcggagtgacccagaatgtgctgtacgagaaccagaagctgatcgccaaccagttcaacagcgccatcggcaagatccaggatagcctgtctagcacagccagcgctctgggcaaactgcaggacgtggtcaatcagaacgctcaggccctgaacaccctcgtgaagcagctgagcagcaatttcggcgccatcagctccgtgctgaacgatatcctgagccggctggataaggtggaagccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggatttctgcggcaagggctaccacctgatgagctttccacagtctgctcctcacggcgtggtgtttctgcacgtgacctatgtgcccgctcaagagaagaacttcacaacagcccctgccatctgccacgacggaaaggcccattttcctagagaaggcgtgttcgtgtccaacggcacccattggttcgtgacacagcggaacttctacgagccccagatcatcaccaccgacaacaccttcgtgtctggcaactgtgacgtcgtgatcggcattgtgaacaacaccgtgtacgaccctctgcagcccgagctggacagcttcaaagaggaactggacaagtactttaagaaccacacaagccccgacgtggacctgggcgatattagcggcatcaatgcctccgtggtcaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgctgcatgaccagctgctgtagctgcctgaagggctgttgcagctgtggcagctgctgcaagttcgacgaggatgatagcgagcctgtgctgaagggcgtgaaactgcactacaccgcggccgc

SEQ ID NO: 9 - protein, 1512 aa, Artificial Sequence
  fusion protein HEV-SARS-CoV-2 spike protein
MIALTLFNLADTLLGGLPTELISSAGGQLFYSRPVVSANGEPTVKLYTSVENAQQDKGIAIPHDIDLGESRVVIQDYDNQHEQDRPTPSPAPSRPFSVLRANDVLWLSLTAAEYDQSTYGSSTGPVYVSDSVTLVNVATGAQAVARSLDWTKVTLDGRPLSTIQQYSKTFFVLPLRGKLSFWEAGTTKAGYPYNYNTTASDQLLVENAAGHRVAISTYTTSLGAGPVSISAVAVLAPHSAFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 10 - protein, 1779 aa, Artificial Sequence
  fusion protein HPV18L1/SARS-CoV-2 spike protein
MALWRPSDNTVYLPPPSVARVVNTDDYVTRTSIFYHAGSSRLLTVGNPYFRVPAGGGNKQDIPKVSAYQYRVFRVQLPDPNKFGLPDTSIYNPETQRLVWACAGVEIGRGQPLGVGLSGHPFYNKLDDTESSHAATSNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPQSLYIKGTGMRASPGSCVYSPSPSGSIVTSDSQLFNKPYWLHKAQGHNNGVCWHNQLFVTVVDTTRSTNLTICASTQSPVPGQYDATKFKQYSRHVEEYDLQFIFQLCTITLTADVMSYIHSMNSSILEDWNFGVPPPPTTSLVDTYRFVQSVAITCQKDAAPAENKDPYDKLKFWNVDLKEKFSLDLDQYPLGRKFLVQAGLRRKPTIGPRKRSAPSATTSSKPAKRVRVRARKFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - protein, 1777 aa, Artificial Sequence
  fusion protein HPV16L1/SARS-CoV-2 spike protein
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQAGLKAKPKFTLGKRKATPTTSSTSTTAKRKKRKLFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 12 - protein, 1407 aa, Artificial Sequence
  fusion protein HBSAg/SARS-CoV-2 spike protein
MNFLGGTTVCLGQNSQSPTSNHSPTSCPPTCPGYRWMCLRRFIIFLFILLLCLIFLLVLLDYQGMLPVCPLIPGSSTTSTGPCRTCTTPAQGTSMYPSCCCTKPSDGNCTCIPIPSSWAFGKFLWEWASARFSFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTAA

SEQ ID NO: 13 - DNA, 665 bp, Artificial Sequence
  RBD SARS-CoV-2 spike protein nucleic acid
gctagcgacgccaccatgagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtgataagcggccgc

SEQ ID NO: 14 - DNA, 665 bp, Artificial Sequence
  RBD SARS-CoV-2 spike protein nucleic acid sequence - human codon optimized
gctagcgacgccaccatgagagtgcagcctacagagtctatcgtgcggttccccaacatcaccaatctgtgccctttcggcgaggtgttcaacgccacaagatttgccagcgtgtacgcctggaaccggaagagaatcagcaactgcgtggccgactacagcgtgctgtacaatagcgccagcttcagcaccttcaagtgctacggcgtgtcccctaccaagctgaacgacctgtgcttcaccaatgtgtacgccgacagcttcgtgatcagaggcgacgaagttcggcagatcgctcctggacagacaggcaagatcgccgattacaactacaagctgcccgacgacttcaccggctgcgtgatcgcctggaatagcaacaacctggacagcaaagtcggcggcaactacaactacctgtaccggctgttccggaagtccaacctgaagcctttcgagcgggacatcagcaccgagatctatcaggccggcagcaccccttgtaatggcgtggaaggcttcaactgctacttcccactgcagtcctacggcttccagcctacaaacggcgtgggctaccagccttatagagtggtggtgctgagcttcgaactgctgcatgcccctgctacagtgtgcggccccaagaagtgataagcggccgc

SEQ ID NO: 15 - protein, 212 aa, SARS-CoV-2
MRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKK

SEQ ID NO: 16 - protein, 7 aa, Artificial Sequence
  EAAAK linker consensus sequence
AEAAAKA

SEQ ID NO: 17 - DNA, 45 bp, Artificial Sequence
  (EAAAK)3 linker
gaagccgccgctaaagaggccgctgccaaagaagctgctgctaag

SEQ ID NO: 18 - protein, 15 aa, Artificial Sequence
  (EAAAK)3 linker
EAAAKEAAAKEAAAK

SEQ ID NO: 19 - protein, 5 aa, Artificial Sequence
  GS linker consensus sequence
GGGGS

SEQ ID NO: 20 - protein, 5 aa, Artificial Sequence
  GS5 linker
GGGGS

SEQ ID NO: 21 - protein, 10 aa, Artificial Sequence
  GS10 linker
GGGGSGGGGS

SEQ ID NO: 22 - DNA, 45 bp, Artificial Sequence
  GS15 linker
ggtggtggtggtagcggtggtggcggttcaggtggcggtggttca

SEQ ID NO: 23 - protein, 15 aa, Artificial Sequence
  GS15 linker
GGGGSGGGGSGGGGS

SEQ ID NO: 24 - protein, 20 aa, Artificial Sequence
  GS20 linker
GGGGSGGGGSGGGGSGGGGS

SEQ ID NO: 25 - protein, 25 aa, Artificial Sequence
  GS25 linker
GGGGSGGGGSGGGGSGGGGSGGGGS

SEQ ID NO: 26 - DNA, 1427 bp, Artificial Sequence
  HBSAg-(EAAAK)3-RBD
gctagcgacgccaccatgattgcactgaccctgtttaatctggcagataccctgttaggtggtctgccgaccgaactgattagcagtgccggtggtcagctgttttatagccgtccggttgttagcgcaaatggtgaaccgaccgttaaactgtataccagcgttgaaaatgcacagcaggataaaggtattgcaattccgcatgatattgatctgggtgaaagccgtgttgtgattcaggattatgataatcagcatgaacaggatcgtccgacaccgagtccggcaccgagccgtccgtttagcgttctgcgtgcaaatgatgttctgtggctgagcctgaccgcagcagaatatgatcagagcacctatggtagcagcaccggtccggtttatgttagcgatagcgttaccctggttaatgttgcaaccggtgcacaggcagttgcacgtagcctggattggaccaaagtgaccctggatggtcgtccgctgagcaccattcagcagtatagcaaaaccttttttgttctgccgctgcgtggtaaactgagcttttgggaagcaggcaccaccaaagcaggttatccgtataactataataccaccgcaagcgatcagctgctggttgaaaacgcagcaggtcatcgtgttgcaattagcacctataccaccagtttaggtgcaggtccggttagcattagcgcagttgcagttctggcaccgcattcagccgaagcagccgctaaagaagcagccgctaaagaagcagccgctaaaagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtgataagcggccgc

SEQ ID NO: 27 - DNA, 1106 bp, Artificial Sequence
  HBSAg-(EAAAK)3-RBD nucleic acid sequence human codon optimised
gctagcgacgccaccatgaattttctcggcggcacaacagtgtgcctgggccagaatagccagtctcctaccagcaatcacagccccaccagctgtcctccaacctgtcctggctacagatggatgtgcctgcggcggttcatcatctttctgttcatcctgctgctgtgcctgatcttcctgctggtgctgctggattaccagggaatgctgcctgtgtgtcctctgatccctggcagcagcacaacaagcacaggcccttgcagaacctgcacaacaccagctcagggcaccagcatgtaccctagctgctgttgtaccaagcctagcgacggcaactgcacatgcatccccattcctagcagctgggccttcggcaagtttctgtgggaatgggccagcgccagattttccgaagccgccgctaaagaggccgctgccaaagaagctgctgctaagagagtgcagcccaccgagtctatcgtgcggttccccaacatcaccaatctgtgccctttcggcgaggtgttcaacgccacaagatttgccagcgtgtacgcctggaaccggaagagaatcagcaactgcgtggccgactacagcgtgctgtacaatagcgccagcttcagcaccttcaagtgctacggcgtgtcccctaccaagctgaacgacctgtgcttcaccaatgtgtacgccgacagcttcgtgatcagaggcgacgaagttcggcagatcgctcctggacagacaggcaagatcgccgattacaactacaagctgcccgacgacttcaccggctgcgtgatcgcctggaatagcaacaacctggacagcaaagtcggcggcaactacaactacctgtaccggctgttccggaagtccaacctgaagcctttcgagcgggacatcagcaccgaaatctaccaggccggcagcaccccttgtaatggcgtggaaggcttcaactgctacttcccactgcagtcctacggcttccagcctacaaacggcgtgggctaccagccttatagagtggtggtgctgagcttcgaactgctgcatgcccctgctacagtgtgcggccccaagaagtgataagcggccgc

SEQ ID NO: 28 - protein, 359 aa, Artificial Sequence
  HBSAg-(EAAAK)3-RBD
MNFLGGTTVCLGQNSQSPTSNHSPTSCPPTCPGYRWMCLRRFIIFLFILLLCLIFLLVLLDYQGMLPVCPLIPGSSTTSTGPCRTCTTPAQGTSMYPSCCCTKPSDGNCTCIPIPSSWAFGKFLWEWASARFSEAAAKEAAAKEAAAKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKK

SEQ ID NO: 29 - DNA, 1418 bp, Artificial Sequence
  HEV-GS15-RBD
gagctcatgattgcactgaccctgtttaatctggcagataccctgctgggtggtctgccgaccgaactgattagcagtgccggtggtcagctgttttatagccgtccggttgttagcgcaaatggtgaaccgaccgttaaactgtataccagcgttgaaaatgcacagcaggataaaggtattgcaattccgcatgatattgatctgggtgaaagccgtgttgtgattcaggattatgataatcagcatgaacaggatcgtccgaccccgagtccggcaccgagccgtccgtttagcgttctgcgtgcaaatgatgttctgtggctgagcctgaccgcagcagaatatgatcagagcacctatggtagcagcaccggtccggtttatgttagcgatagcgttaccctggttaatgttgcaaccggtgcacaggcagttgcacgtagcctggattggaccaaagtgaccctggatggtcgtccgctgagcaccattcagcagtatagcaaaaccttttttgttctgccgctgcgtggtaaactgagcttttgggaagcaggcaccaccaaagcaggttatccgtataactataataccaccgcaagcgatcagctgctggttgaaaacgcagcaggtcatcgtgttgcaattagcacctataccaccagtctgggtgcaggtccggttagcattagcgcagttgcagttctggcaccgcatagcgcaggtggaggaggttctggaggcggtggaagtggtggcggaggtagcagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtgataagcggccgc

SEQ ID NO: 30 - DNA, 1418 bp, Artificial Sequence
  HEV-GS15-RBD nucleic acid sequence optimized for E.coli expression
gagctcatgattgcactgaccctgtttaatctggcagataccctgttaggtggtctgccgaccgaactgattagcagtgccggtggtcagctgttttatagccgtccggttgttagcgcaaatggtgaaccgaccgttaaactgtataccagcgttgaaaatgcacagcaggataaaggtattgcaattccgcatgatattgatctgggtgaaagccgtgttgtgattcaggattatgataatcagcatgaacaggatcgtccgacaccgagtccggcaccgagccgtccgtttagcgttctgcgtgcaaatgatgttctgtggctgagcctgaccgcagcagaatatgatcagagcacctatggtagcagcaccggtccggtttatgttagcgatagcgttaccctggttaatgttgcaaccggtgcacaggcagttgcacgtagcctggattggaccaaagtgaccctggatggtcgtccgctgagcaccattcagcagtatagcaaaaccttttttgttctgccgctgcgtggtaaactgagcttttgggaagcaggcaccaccaaagcaggttatccgtataactataataccaccgcaagcgatcagctgctggttgaaaacgcagcaggtcatcgtgttgcaattagcacctataccaccagtttaggtgcaggtccggttagcattagcgcagttgcagttctggcaccgcattcagccggtggtggtggtagcggtggtggcggttcaggtggcggtggttcacgtgttcagccgacagaaagcattgttcgttttccgaatatcaccaatctgtgtccgtttggcgaagtttttaatgcaacccgttttgcaagcgtttatgcctggaatcgtaaacgtattagcaattgcgttgccgattatagcgtgctgtataatagcgcaagctttagcacctttaaatgctatggtgttagcccgaccaaactgaatgatctgtgttttaccaatgtgtatgccgatagctttgtgattcgtggtgatgaagttcgtcagattgcaccgggtcagaccggtaaaattgcagattataactacaaactgccggatgattttacgggttgtgttattgcatggaatagcaataacctggatagcaaagttggtggcaactataactatctgtatcgcctgtttcgtaagagcaatctgaaaccgtttgaacgtgatattagcaccgaaatttatcaggcaggtagcaccccgtgcaatggtgttgaaggttttaattgttattttccgctgcagagctatggttttcagcctaccaatggtgtgggttatcagccgtatcgtgttgttgttctgtcatttgaactgctgcatgcaccggcaaccgtttgtggtccgaaaaaatgataagcggccgc

SEQ ID NO: 31 - protein, 466 aa, Artificial Sequence
  HEV-GS15-RBD
MIALTLFNLADTLLGGLPTELISSAGGQLFYSRPVVSANGEPTVKLYTSVENAQQDKGIAIPHDIDLGESRVVIQDYDNQHEQDRPTPSPAPSRPFSVLRANDVLWLSLTAAEYDQSTYGSSTGPVYVSDSVTLVNVATGAQAVARSLDWTKVTLDGRPLSTIQQYSKTFFVLPLRGKLSFWEAGTTKAGYPYNYNTTASDQLLVENAAGHRVAISTYTTSLGAGPVSISAVAVLAPHSAGGGGSGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKK

SEQ ID NO: 32 - DNA, 4566 bp, Artificial Sequence
  HBSAg-(EAAAK)3-full-length SARS-CoV-2 spike protein nucleic acid sequence human codon optimised
aagcttgccgccaccatggagaacatcacatcaggattcctaggacccctgctcgtgttacaggcggggtttttcttgttgacaagaatcctcacaataccacagagtctagactcgtggtggacttctctcaattttctagggggatcacccgtgtgtctgggccaaaattcgcagtccccaacctccaatcactcaccaacctcttgtcctccaatttgtcctggctatcgctggatgtgtctgcggcgttttatcatattcctcttcatcctgctgctatgcctcatcttcttgttggttcttctggactaccagggtatgttgcccgtttgtcctctaattccaggatcaacaactaccaacacgggaccatgcaagacctgcacgactcctgctcaaggaaactctatgtttccctcttgttgctgtacaaaacctaccgacggaaactgcacttgtattcccatcccatcatcctgggctttcgcaaaatacctatgggagtgggcctcagtccgtttctcctggctcagtttactagtgccatttgttcagtggttcgtagggctttcccccactgtttggctttccgctatatggatgatgtggtattgggggccaagtctgtacagcatcgtgagtccctttatacctctattaccaattttcttttgtctttgggtatacattgaggctgccgcaaaggaagccgcagctaaagaggcagctgccaagttcgtgttcctggttctgctgcccctggtgtctagccagtgcgtgaacctgaccaccagaacacagctgcctccagcctacaccaacagcttcaccagaggcgtgtactaccccgacaaggtgttccggtcctccgtgctgcattctacccaggacctgttcctgcctttcttctccaacgtgacctggttccacgccatccatgtgtctggcaccaacggcaccaagagattcgacaaccccgtgctgcctttcaacgacggggtgtactttgcctccaccgagaagtccaacatcatcagaggctggatcttcggcacaaccctggacagcaagacccagagcctgctgatcgtgaacaacgccaccaacgtggtcatcaaagtgtgcgagttccagttctgcaacgaccccttcctgggcgtctactaccacaagaacaacaagtcctggatggaatccgagttccgggtgtactcctccgccaacaactgcaccttcgaatacgtgtcccagcctttcctgatggacctggaaggcaagcagggcaacttcaagaacctgcgcgagttcgtgttcaagaacatcgacggctacttcaagatctactccaagcacacccctatcaacctcgtgcgggatctgcctcagggcttctctgctctggaacccctggtggatctgcccatcggcatcaacatcacccggtttcagaccctgctggccctgcaccggtcttatttgacccctggcgactcctcttctggctggactgctggcgccgctgcttactatgtgggctacctgcagcctcggacctttctgctgaagtacaacgagaatggcaccatcaccgacgccgtggactgtgctctggatcctctgtccgagacaaagtgcaccctgaagtccttcaccgtggaaaagggcatctaccagacctccaacttccgggtgcagcccaccgagtctatcgtgcggttccctaacatcaccaacctgtgtcctttcggcgaggtgttcaatgccaccagattcgcctctgtgtacgcctggaaccggaagcggatctctaactgcgtggccgactacagcgtgctgtacaactccgcctccttcagcaccttcaagtgctacggcgtgtcccctacaaagctgaacgacctgtgcttcacaaacgtgtacgccgacagcttcgtgatccggggagatgaagtgcggcagatcgctcctggacagaccggcaagatcgccgattacaactacaagctgcccgacgacttcaccggctgtgtgatcgcttggaactccaacaacctggactccaaagtcggcggcaactacaactacctgtaccggctgttccggaagtctaacctgaagcctttcgagcgggacatcagcaccgagatctaccaggctggcagcaccccttgtaacggcgtggaaggcttcaactgctacttcccactgcagtcctacggctttcagcctaccaatggcgtgggctatcagccctacagagtggtggtgctgtccttcgagctgctgcatgctcctgctaccgtgtgcggccctaagaaatctaccaacctggtcaagaacaaatgcgtgaacttcaacttcaacggcctgaccggcaccggcgtgctgacagagtccaacaagaagttcctgccattccagcagttcggccgggatatcgccgataccacagatgccgtcagggaccctcagacactggaaatcctggacatcaccccttgctccttcggcggagtgtctgtgatcaccccaggcaccaacacctctaaccaggtggccgtgctgtatcaggacgtgaactgtaccgaggtgcccgtggctatccatgccgatcagctgacccctacatggcgcgtgtactccaccggctctaacgtgttccagacaagagctggctgtctgatcggcgctgagcacgtgaacaattcctacgagtgcgacatccccatcggagccggaatctgcgcctcttatcagacccagaccaactctcccagacgggccagatctgtggccagccagtctatcattgcttacaccatgagcctgggcgccgagaactctgtggcctacagcaacaactctatcgctatccccaccaacttcaccatctccgtgaccacagagatcctgccagtgtccatgaccaagaccagcgtggactgcaccatgtacatctgcggcgactctaccgagtgctccaacctgctgctccagtacggctccttctgcacccagctgaatagagccctgaccggaatcgccgtggaacaggacaagaacacccaagaggtgttcgcccaagtgaagcagatctacaagacccctcctatcaaggacttcggcggcttcaatttctcccagattctgcccgatcctagcaagccctccaagcggtctttcatcgaggacctgctgttcaacaaagtgacactggccgacgccggcttcatcaagcagtacggcgactgtctgggcgacattgccgctagggatctgatctgcgcccagaagtttaacggactgacagtgctgcctcctctgctgaccgatgagatgatcgcccagtacacctccgcactgctggctggcacaatcacctctggatggacatttggcgctggcgctgctctgcaaatcccattcgctatgcaaatggcctaccggttcaacggcatcggcgtgacccagaatgtgctgtacgagaaccagaagctgatcgccaaccagttcaacagcgccatcggaaagatccaggacagcctgtccagcaccgcttctgccctgggaaagctgcaggatgtggtcaaccagaacgctcaggccctgaacaccctcgtgaagcagctgtctagcaacttcggcgccatctcctctgtgctgaacgatatcctgagccggctggacaaggtggaagccgaggtgcagatcgacagactgatcaccggacggctgcagtccctgcagacctatgttacccagcagctgatccgggctgccgagattagagcctctgccaatctggccgcaaccaagatgtctgagtgtgtgctgggacagtccaagagagtggacttctgcggcaagggctaccacctgatgagcttccctcagtctgctcctcacggcgtggtgtttctgcacgtgacctacgtgcccgctcaagagaagaactttaccaccgctcctgccatctgccacgacggcaaggctcactttcctagagaaggcgtgttcgtgtctaacggcacccattggttcgtgacacagcggaacttctacgagccccagatcatcaccaccgacaacaccttcgtgtccggcaactgcgacgtcgtgatcggaattgtgaacaataccgtgtacgaccctctgcagcccgagctggactccttcaaagaggaactggacaagtactttaagaaccacacaagccccgacgtggacctgggagacatctctggcatcaacgcctccgtggtcaacatccagaaagagatcgaccggctgaacgaggtggccaagaatctgaacgagtccctgatcgacctgcaagaactggggaagtacgagcagtacatcaagtggccctggtacatctggctgggctttatcgctggcctgatcgctatcgtgatggtcacaatcatgctgtgctgtatgacctcctgttgctcctgcctgaagggctgctgctcttgcggctcttgctgcaagttcgacgaggacgactctgagcccgtgctgaaaggcgtgaagctgcactatacctgatgactcgag

SEQ ID NO: 33 - protein, 1513 aa, Artificial Sequence
  HBSAg-(EAAAK)3-full-length SARS-CoV-2 spike protein
MENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYIEAAAKEAAAKEAAAKFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT